(19) United States
(12) Patent Application Publication
(10) Pub. No.: US 2024/0301492 A1
(43) Pub. Date: Sep. 12, 2024

(54) METHODS OF PREPARING ASSAYS, SYSTEMS, AND COMPOSITIONS FOR DETERMINING FETAL FRACTION

(71) Applicant: Enumera Molecular, Inc., Ann Arbor, MI (US)

(72) Inventors: Amin R. Mazloom, Del Mar, CA (US); Kevin Jacobs, Ann Arbor, MI (US); Vladislav Volman, San Diego, CA (US); Jeffrey William Perry, Plymouth, MI (US); Ronald David Beaubien, Jr., Jackson, MI (US)

(21) Appl. No.: 18/258,516

(22) PCT Filed: Dec. 22, 2021
(86) PCT No.: PCT/US21/64916

§ 371 Date: Jun. 20, 2023

Related U.S. Application Data
(60) Provisional application No. 63/130,543, filed on Dec. 24, 2020.

(51) Int. Cl. C12Q 1/6881; C12Q 1/6874; G16B 30/00; G16B 40/20

(52) U.S. Cl. CPC C12Q 1/6881; C12Q 1/6874; G16B 30/00; G16B 40/20; C12Q 2600/16

(57) ABSTRACT

Methods, systems, and kits for estimating fetal fraction in a mixed fetal-maternal DNA sample are disclosed, by molecular counting of a predetermined set of informative sequences. These find use in detecting and quantifying variations in gene dosage, e.g., due to gene duplication, or to variations from the normal euploid complement of chromosomes, e.g., trisomy of one or more chromosomes that are normally found in diploid pairs, in the context of non-invasive prenatal testing.

The present application claims priority to U.S. Provisional Application Ser. No. 63/130,543, filed Dec. 24, 2020, which is incorporated herein by reference.

### FIELD OF THE INVENTION

The present invention relates to systems and methods for determining the fraction of fetal DNA in a mixed sample comprising maternal and fetal DNA. In some embodiments, the fraction of fetal DNA can be determined without whole-genome/whole-exome sequencing, and in some preferred embodiments, without digital sequencing. The technologies find application in prenatal testing, particularly for non-invasive prenatal testing (NIPT). NIPT is directed to the analysis of cell-free DNA (cfDNA) from a fetus that circulates in the blood of a woman carrying the fetus in utero. Analysis of cell-free DNA in maternal blood can be used to assess the health of the fetus. Estimation of the fetal fraction within such a sample may improve the accuracy of the assessment, particularly in the context of analyzing copy number variations of various sizes (e.g., aneuploidies). Thus, the technology herein relates to methods, systems, and kits for detecting and quantifying variations in copy number of portions of the genome (e.g., departure from the expected diploid representation of a portion of the genome forming part of an autosome or X chromosome in a female fetus or monoploid representation of a portion of the genome forming part of the Y chromosome), from gene dosage, (e.g., due to gene duplication), to variations from the normal euploid complement of chromosomes, (e.g., trisomy of one or more chromosomes that are normally found in diploid pairs), in a mixed sample comprising maternal and fetal DNA, comprising estimation of the fetal fraction in the sample.

### BACKGROUND OF THE INVENTION

Chromosomal abnormalities can affect either the number or structure of chromosomes. Conditions wherein cells, tissues, or individuals have one or more whole chromosomes or segments of chromosomes either absent, or in addition to the normal euploid complement of chromosomes can be referred to as aneuploidy. Germline replication errors due to chromosome non-disjunction result in either monosomies (one copy of an autosomal chromosome instead of the usual two or only one sex chromosome) or trisomies (three copies). Such events, when they do not result in outright embryonic demise, typically lead to a broad array of disorders often recognized as syndromes, e.g., trisomy 21 and Down's syndrome, trisomy 18 and Edward's syndrome, and trisomy 13 and Patau's syndrome. Structural chromosome abnormalities affecting parts of chromosomes arise due to chromosome breakage, and result in deletions, inversions, translocations or duplications of large blocks of genetic material. These events are often as devastating as the gain or loss of the entire chromosome and can lead to such disorders as Prader-Willi syndrome (del 15q11-13), retinoblastoma (del 13q14), Cri du chat syndrome (del 5p), and others listed in U.S. Pat. No. 5,888,740, herein incorporated in its entirety by reference.

Major chromosomal abnormalities are detected in nearly 1 of 140 live births and in a much higher fraction of fetuses that do not reach term or are still-born (Hsu (1998) Prenatal diagnosis of chromosomal abnormalities through amniocentesis. In: Milunsky A, editor. Genetic Disorders and the Fetus. 4 ed. Baltimore: The Johns Hopkins University Press. 179-180; Staebler et al. (2005) “Should determination of the karyotype be systematic for all malformations detected by obstetrical ultrasound?” Prenat Diagn 25: 567-573). The most common aneuploidy is trisomy 21 (Down syndrome), which currently occurs in 1 of 730 births (Hsu (2008); Staebler et al. (2005)). Though less common than trisomy 21, trisomy 18 (Edwards Syndrome) and trisomy 13 (Patau syndrome) occur in 1 in 5,500 and 1 in 17,200 live births, respectively (Hsu (2008)). A large variety of congenital defects, growth deficiencies, and intellectual disabilities are found in children with chromosomal aneuploidies, and these present life-long challenges to families and societies (Jones (2006) Smith's recognizable patterns of human malformation. Philadelphia: Elsevier Saunders).

There are a variety of prenatal tests that can indicate increased risk for fetal aneuploidy, including invasive diagnostic tests such as amniocentesis or chorionic villus sampling, which are the current gold standard but are associated with a non-negligible risk of fetal loss (American College of Obstetricians and Gynecologists (2007) ACOG Practice Bulletin No. 88, December 2007. Invasive prenatal testing for aneuploidy. Obstet Gynecol 110: 1459-1467). More reliable, non-invasive tests for fetal aneuploidy have therefore long been sought. The most promising of these are based on the detection of fetal DNA in maternal plasma. It has been demonstrated that massively parallel sequencing of libraries generated from maternal plasma can reliably detect chromosome 21 abnormalities (see, e.g., Chiu et al., Noninvasive prenatal diagnosis of fetal chromosomal aneuploidy by massively parallel genomic sequencing of DNA in maternal plasma. Proc Natl Acad Sci USA 105:20458-20463 (2008); Fan et al., Noninvasive diagnosis of fetal aneuploidy by shotgun sequencing DNA from maternal blood. Proc Natl Acad Sci USA 105: 16266-16271 (2008); U.S. Pat. No. 7,888,017).

A major challenge associated with noninvasive prenatal diagnosis of fetal aneuploidy is the fact that fetal DNA represents a small proportion of the total cell-free DNA (cfDNA) in maternal plasma. This proportion is referred to as the “fetal fraction” (FF). It is typically between 5-15%, and varies from pregnancy to pregnancy as well as during the course of a pregnancy (Hui & Blanchi (2020), Fetal fraction and noninvasive prenatal testing: What clinicians need to know. Prenatal Diagnosis; 40: 155-163. https://doi.org/10.1002/pd.5620). FIG. 1 illustrates an empirical distribution of the fetal fraction in maternal blood samples, obtained by the inventors, showing the relatively widespread distribution of fetal fraction. Fetal fraction is an important sample quality control parameter for NIPT tests and can influence statistical confidence in any result thereof. Indeed, knowing the fetal fraction ensures that the amount of fetal DNA in the sample was sufficient for meaningful results to be obtained, as well as providing important information on the assessment of the statistical significance of a deviation from the expected number of copies of a portion of the genome (e.g., for detection of aneuploidies) (Hui & Blanchi (2020)). Approaches for estimating the FF have been suggested, based on the detection of polymorphic loci that can differentiate between the mother and the fetus (see e.g., Sparks et al. (2012) Noninvasive prenatal detection and selective analysis of cell-free DNA obtained from maternal blood: evaluation for trisomy 21 and trisomy 18. American Journal of Obstetrics & Gynecology, vol. 206, issue 4, P319.ET-319.E9), the size distribution of DNA fragments in the sample (as fetal fragments are typically shorter than maternal ones; see e.g., Yu et al. (2014) Size-based molecular diagnostics using plasma DNA for noninvasive prenatal testing. Proc Natl Acad Sci USA. 2014 Jun. 10; 111(23):8583-8. doi: 10.1073/pnas.1406103111) or the size and locations of the starts and ends of fragments identified (see e.g., Sun et al. (2018) Size-tagged preferred ends in maternal plasma DNA shed light on the production mechanism and show utility in noninvasive prenatal testing. PNAS May 29, 2018 115 (22) E5106-E5114). These approaches all have drawbacks. The use of polymorphic loci requires a detecting technology that can discriminate between alleles at those informative positions (such as e.g., sequencing or specifically designed SNP microarrays), while approaches that rely on fragment lengths and end characteristics typically require whole genome massively parallel sequencing.

Current methods for quantifying variations in numbers of molecules, for example performing aneuploidy screening, that rely on next generation sequencing (NGS) or SNP microarrays for quantification and estimation of the fetal fraction, are often time-consuming, expensive, and require extensive bioinformatics analysis.

### SUMMARY OF THE INVENTION

The present invention provides compositions, methods, and systems for the estimation of fetal DNA fraction in mixed fetal-maternal samples by counting particular nucleic acid molecules that may be represented in the samples. The technology finds application, for example, in analyzing genetic variations, including but not limited to alterations in copy number such as, e.g., genomic deletions or insertions of various sizes including aneuploidy, in mixed fetal-maternal samples. In various preferred embodiments, the technology uses methods for detecting and thereby counting single copies of target nucleic acid molecules, without the use of “next generation” sequencing (NGS) technologies, such as those described by Chiu et al. and Fan, et al., supra. Indeed, the present inventors have identified that it was possible to estimate the fetal DNA fraction in a mixed fetal-maternal sample using molecular counts of targeted nucleic acid molecules from predetermined genomic regions, where the amount of molecules identified as originating from these regions correlates with the fetal fraction in the sample. While it had been previously speculated that data from whole genome sequencing could be used to obtain estimates of fetal fraction by characterizing one or more genome-wide features related to the location, allelic proportions, and/or length of the fragments sequenced, the inventors have for the first time shown that it was possible to obtain useful fetal fraction information from molecular counts from a predetermined set of specific genomic regions without measurement of DNA fragment size or allelic proportions. In other words, the inventors have discovered that the molecular counts from specific genomic regions are associated with different patterns as a function of fetal fraction, that reflect underlying biological differences. The inventors have further demonstrated that these patterns can be detected and exploited to infer fetal fraction in a mixed fetal-maternal sample. As the skilled person understands, this is a major conceptual leap from prior methods. It is not necessary to characterize (e.g., by genotyping or sequencing) a large number of polymorphic alleles, quantitatively measure DNA fragment length distributions, or to perform a genome wide unspecific survey (where sequencing data may be mapped to specific regions of the genome, some of which may be subsequently identified as informative for a particular sample) to the specific interrogation of predetermined regions known to be reliably informative across samples.

Thus, the compositions, methods, and systems can be used to determine fetal fraction information from molecular counts without complex sequencing or genotyping assays. These compositions, methods, and systems can be used alone or in conjunction with other assays to improve the detection or characterization of fetal DNA in a mixed maternal-fetal sample, including e.g., genomic deletions and duplications of various sizes, including complete chromosomes, arms of chromosomes, microscopic deletions and duplications, submicroscopic deletions and deletions, and single nucleotide features, including single nucleotide polymorphisms, deletions, and insertions. The methods find particular use in noninvasive prenatal testing (both qualitative and quantitative genetic testing, such as detecting Mendelian disorders, insertions/deletions, and chromosomal imbalances).

In some embodiments, the technology herein uses methods for characterizing cell-free DNA (cfDNA), for example, circulating cfDNA from blood or plasma, in a sequence-specific and quantitative manner. In some embodiments, single copies of the DNA are detected and counted, without polymerase chain reaction or DNA sequencing. Embodiments of the technology use methods, compositions, and systems for detecting target DNA using methods for amplifying signals that are indicative of the presence of the target DNA in the sample. In various embodiments, the detectable signal from a single target molecule is amplified to such an extent and in such a manner that the signal derived from the single target molecule is detectable and identifiable, in isolation from signal from other targets and from other copies of the target molecule.

Embodiments of the technology use methods for counting products formed by rolling circle replication, e.g., in a rolling circle amplification (RCA) reaction. In some embodiments the technology uses methods of counting RCA product molecules formed by replication from circularized nucleic acid probe molecules, e.g., molecular inversion probes (MIPs), including, e.g., padlock probes. Circularized nucleic acid probes may be formed, for example, by hybridization of a linear probe molecule having unique polynucleotide arms designed to hybridize immediately upstream and downstream of a specific target sequence (or site) in a nucleic acid target, e.g., in an RNA, cfDNA, or genomic nucleic acid sample and ligating the arms together to form a circularized nucleic acid probe. In some embodiments a MIP probe forms a ligatable nick upon hybridization to the nucleic acid target, while in some embodiments, the MIP probe is modified or repaired (e.g., by gap filling, flap cleavage, etc.) to form a nick prior to ligation. In various embodiments of the invention described, a number or amount of circularized nucleic acid probes formed in a reaction mixture is indicative of a number or amount of target nucleic acids in the reaction mixture.

### Definitions

To facilitate an understanding of the present invention, a number of terms and phrases are defined below:

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, the terms “computer system” includes the hardware, software and data storage devices for embodying a system or carrying out a method according to the above-described embodiments. For example, a computer system may comprise a central processing unit (CPU), input means, output means and data storage, which may be embodied as one or more connected computing devices. In various embodiments, the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

As used herein, the terms “subject” and “patient” refer to any animal (e.g., mammals such as dogs, cats, livestock, and humans). In some embodiments, the subject or patient is a human.

The term “sample” in the present specification and claims is used in its broadest sense and refers to any material comprising nucleic acids. Biological samples may be animal, including human, fluid, solid (e.g., stool) or tissue. Biological samples may be obtained from all of the various families of domestic animals, as well as feral or wild animals, including, but not limited to, such animals as canines, felines, ungulates, bear, fish, lagomorphs, rodents, marsupials, etc. Particularly preferred sources of target nucleic acids are biological samples including, but not limited to blood, plasma, and serum.

The term “mixed sample” refers to a sample comprising a mixture of maternal and fetal DNA. In some embodiments, both the maternal and fetal DNA are cell free DNA (cfDNA). A mixed sample may be a maternal blood sample, or a sample derived therefrom, such as e.g., a plasma or serum sample, or a purified cell free DNA sample. A mixed sample may also be an artificial sample, for example obtained by combining known proportions of fetal and maternal DNA.

The term “fetal fraction” (FF) refers to the proportion of fetal DNA in a mixed sample comprising both fetal and maternal DNA. The fetal fraction is a unitless metric with values between 0 and 1 (or between 0 and 100%), typically between 0 and 0.2 (0 and 20%).

The term “informative region” or “informative site” refers to a genomic region that has a different likelihood of being identified in fetal DNA and in maternal DNA in a mixed sample. As a result, the amount of DNA from an informative region in a mixed sample is dependent on the fetal fraction in the sample. Conversely, the term “uninformative region” or “unenriched region” refers to a genomic region that does not have a different likelihood of being identified in fetal DNA and in maternal DNA in a mixed sample. Informative regions may be identified as regions that are such that molecular counts from mixed samples comprising fetal and maternal DNA for these regions are significantly associated with fetal fraction according to a statistical model. Uninformative regions may be identified as regions that are such that molecular counts from mixed samples comprising fetal and maternal DNA for these regions are not significantly associated with fetal fraction according to a statistical model. An informative region may also be referred to as a “maternally enriched region”, when the amount of DNA from the informative region in a mixed sample is negatively associated with the fetal fraction in the sample. An informative region may also be referred to as a “fetally enriched region”, when the amount of DNA from the informative region in a mixed sample is positively associated with the fetal fraction in the sample. According to the present invention, preferred informative regions are maternally enriched regions, as such regions tend to occur more frequently and with larger enrichment effect size. This may be due to an excess of genomic regions with open chromatin in trophoblast cell which are bound to closed chromatin in maternally derived cells. An association between the amount of DNA from a region in a mixed sample and the fetal fraction in the sample may be identified using a statistical model applied to molecular counts associated with the region. The statistical model may be a regression model, such as e.g., a regression model or a generalized linear model, which models the molecular counts from a region as a function of fetal fraction. For example, this may take the form of a model Yᵢ=f(X,θ,f)+εᵢ where Yᵢ represents the molecular counts from one or more regions, or a metric derived therefrom (e.g., a summarized and/or fractional count), X is a design matrix with terms obtained from training data, θ is a vector of parameters estimated from the model, f is an estimate of the fetal fraction, and si is an error term. In such models, the strength of association between a region and the fetal fraction may be identified as a parameter in the model (e.g., parameter R in the formulation above), and the significance of the association may be assessed by quantifying the statistical significance of the parameter estimate. The statistical model may be a correlation model between the molecular counts from a region and the fetal fraction, such as e.g., a Pearson correlation or a Spearman rank correlation. In such models, the strength of association between a region and the fetal fraction may be identified as the value of the correlation coefficient, and the significance of the association may be assessed by quantifying the statistical significance of the correlation coefficient estimate. The statistical model may model the expected molecular count for a region in the genome as the product of: the total number of counts obtained from a mixed sample from sites with known ploidy, and a region enrichment factor that is expressed as a weighted combination of a maternal enrichment factor, with weight equal to (1-fetal fraction) and a fetal enrichment factor, with weight equal to the fetal fraction. In some such embodiments, the expected molecular count for a region in the genome may be assumed to have a Poisson distribution, a negative binomial distribution, a normal distribution, a distribution from the exponential family, an empirical distribution, or a non-parametric distribution. Informative regions may be identified using such a statistical model by fitting the model to training data and testing whether the site-specific fetal enrichment factor and the site-specific maternal enrichment factor estimated for the region are significantly different. In some such embodiments, the strength of association between a region and the fetal fraction may be identified as the difference or absolute difference between the site-specific fetal enrichment factor and the site-specific maternal enrichment factor for the region.

Alternatively, the strength of association between a region and the fetal fraction may be identified as the ratio between the site-specific fetal enrichment factor and the site-specific maternal enrichment factor for the region (also referred to herein as “enrichment ratio” or “fetal enrichment ratio”). Negative predictor regions may be associated with enrichment ratios that are below 1 (or significantly below 1). Positive predictor regions may be associated with enrichment ratios that are above 1 (or significantly above 1). Uninformative regions may be associated with enrichment ratios that are approximately equal to 1 (or not significantly different from 1). In some such embodiments, the significance of the association may be assessed by quantifying the statistical significance of the enrichment ratio or difference between the site-specific fetal enrichment factor and the site-specific maternal enrichment factor for the region.

As used herein, the term “statistical significance” refers to any metric quantifying the certainty of a test result according to a particular statistical model. As the skilled person understand, a result has statistical significance when it is very unlikely to have occurred given a null hypothesis. In the context of the present invention, a null hypothesis may be formulated to capture the assumption that the molecular counts from a genomic region in a mixed sample are not significantly associated with fetal fraction. This may take the form of e.g., a correlation between the molecular counts and fetal fraction being 0, the gain in a linear model of fetal fraction as a function of molecular counts being 0, or the difference between a site-specific fetal enrichment factor and the site-specific maternal enrichment factor for the region being 0. A region may be considered to be significantly associated with fetal fraction when the null hypothesis can be rejected with at least a predetermined level of confidence. A predetermined level of confidence may be expressed as a threshold on the p-value associated with the test. Thresholds such as p-value <0.05, <0.01, <0.005, <0.001 are commonly used.

The term “cell free DNA” (or “cell-free DNA”, “cfDNA”, “circulating free DNA”) refers to DNA fragments that are circulating in bodily fluids such as blood, or purified versions thereof such as serum or plasma, urine, cerebrospinal fluid, etc. Within the context of the present invention, a sample comprising cell free DNA is typically a blood sample or a sample derived from a blood sample, such as e.g., a plasma or serum sample. In various embodiments, the sample is a sample of maternal blood, comprising both maternal and fetal circulating cell free DNA. Fetal circulating cell free DNA fragments may be derived from fetal or placental tissue that are circulating in the blood of expectant mothers.

The term “target” as used herein refers to a molecule sought to be sorted out from other molecules for assessment, measurement, or other characterization. For example, a target nucleic acid may be sorted from other nucleic acids in a sample, e.g., by probe binding, amplification, isolation, capture, etc. When used in reference to a hybridization-based detection, e.g., polymerase chain reaction, “target” refers to the region of nucleic acid bounded by the primers used for polymerase chain reaction, while when used in an assay in which target DNA is not amplified, e.g., in capture by molecular inversion probes (MIPS), a target comprises the site bounded by the hybridization of the target-specific arms of the MIP, such that the MIP can be ligated and the presence of the target nucleic acid can be detected.

The term “targeted” in relation to any technology or protocol refers to the technology or protocol being designed to measure or characterize (in particular, count) a specific target or sets of targets. Within the context of the present invention, a target is typically a nucleic acid defined by its sequence. Thus, a targeted technology or protocol is one that is designed to characterize a sample in terms of its content of nucleic acids that have a predetermined sequence or sets of sequences. As an example, a protocol that involves capture of specific sequences (e.g., using molecular inversion probes) followed by next generation sequencing of the captured material is a targeted protocol. By contrast, a protocol that sequences all of the genetic material present in a sample without any sequence specific capture step (whole genome sequencing) is not a targeted protocol. Similarly, an array based protocol that is designed to detect sequences from an entire genome or portion of the genome by tiling said genome or portion of genome is not a targeted protocol. By contrast, an array based protocol that is designed to specifically detect sequences from predetermined regions of a genome is a targeted protocol.

The term “molecular count” refers to any measurable quantity that is representative of the amount of a target within a sample. For example, in the context of a sample of cell free DNA, a target may be a particular DNA sequence, and a molecular count may be any measurable quantity that is representative of the amount of cfDNA in the sample that comprises the target DNA sequence. A molecular count may in practice be an absolute value or a relative value (in which case it may also be referred to as a fractional count). A molecular count for a target nucleic acid may be obtained using any nucleic acid detection assay known in the art, including e.g., sequencing (in which case the molecular count may be referred to as “read count”), a combined labeling and imaging technique (see e.g., F. Dahl, et al., Imaging single DNA molecules for high precision NIPT; Nature Scientific Reports 8:4549 (2018) p1-8), a microarray, etc. In particular embodiments, a molecular count may be obtained by counting the products of a rolling circle amplification as further described herein, and as described in WO 2019/195346 A1 to Sekedat, et al. (Methods, Systems, and Compositions for Counting Nucleic Acids (2019)). A molecular count for a genomic region may be obtained a combined count of target molecules that are associated with the region. For example, molecular counts for any target nucleic acid that maps within the region may be included in the molecular count for the region. Thus, the molecular count for a genomic region may in particular not be dependent on the particular start and/or end location of target nucleic acids within a genomic region, as long as the target nucleic acids map within the genomic region.

The term “genomic region” as used herein refers to a region of the genome of a subject. A genomic region may be specified using genomic coordinates in a reference genome. Suitably, a genomic region may be specified using coordinates in a reference genome available from the Genome Reference Consortium. For example, when the subject is a human, a genomic region may be specified using coordinates in the GRCh38 reference genome, available at world wide web at ncbi.nlm.nih.gov/grc/human.

The term “copy number” as used herein refers to the copy number of a gene, a genic region (also referred to as “gene dosage”), a chromosome, or fragments or portions thereof. Normal individuals carry two copies of most genes or genic regions, one on each of two chromosomes. However, there are certain exceptions, e.g., when genes or genic regions reside on the X or Y chromosomes, or when genes sequences are present in pseudogenes or segments of the genome present with variable copy number.

The term “aneuploidy” as used herein refers to conditions wherein cells, tissues, or individuals have one or more whole chromosomes or segments of chromosomes either absent, or in addition to the normal euploid complement of chromosomes.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full-length coding sequence or by any portion of the coding sequence so long as the desired activity or function is retained.

The term “genic region” as used herein refers to a gene, its exons, its introns, and its regions flanking it upstream and downstream, e.g., 5 to 10 kilobases 5′ and 3′ of the transcription start and stop sites, respectively.

The term “genic sequence” as used herein refers to the sequence of a gene, its introns, and its regions flanking it upstream and downstream, e.g., 5 to 10 kilobases 5′ and 3′ of the transcription start and stop sites, respectively.

The term “chromosome-specific” as used herein refers to a sequence that is found only in that particular type of chromosome.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology.

The term “oligonucleotide” as used herein is defined as a molecule comprising two or more deoxyribonucleotides or ribonucleotides, in some embodiments the oligonucleotide has at least 5 nucleotides, in other embodiments the oligonucleotide has at least about 10-15 nucleotides and in yet other embodiments the oligonucleotide has at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated, e.g., in the presence of nucleotides and a suitable nucleic acid polymerase. An oligonucleotide “primer” may occur naturally, may be made using molecular biological methods, e.g., purification of a restriction digest, or may be produced synthetically. In preferred embodiments, a primer is composed of or comprises DNA.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene. Multiple sequence variants for a genomic location may be referred to as “alleles”.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include base analogs, and comprise modified forms of deoxyribonucleotides as well as ribonucleotides, and include but are not limited to modified bases and nucleotides described in U.S. Pat. Nos. 5,432,272; 6,001,983; 6,037,120; 6,140,496; 5,912,340; 6,127,121 and 6,143,877, each of which is incorporated herein by reference in their entireties; heterocyclic base analogs based on the purine or pyrimidine ring systems, and other heterocyclic bases.

The term “continuous strand of nucleic acid” as used herein is means a strand of nucleic acid that has a continuous, covalently linked, backbone structure, without nicks or other disruptions. The disposition of the base portion of each nucleotide, whether base-paired, single-stranded or mismatched, is not an element in the definition of a continuous strand. The backbone of the continuous strand is not limited to the ribose-phosphate or deoxyribose-phosphate compositions that are found in naturally occurring, unmodified nucleic acids. A nucleic acid of the present invention may comprise modifications in the structure of the backbone, including but not limited to phosphorothioate residues, phosphonate residues, 2′ substituted ribose residues (e.g., 2′-O-methyl ribose) and alternative sugar (e.g., arabinose) containing residues.

The term “continuous duplex” as used herein refers to a region of double stranded nucleic acid in which there is no disruption in the progression of base pairs within the duplex (i.e., the base pairs along the duplex are not distorted to accommodate a gap, bulge or mismatch with the confines of the region of continuous duplex). As used herein the term refers only to the arrangement of the base pairs within the duplex, without implication of continuity in the backbone portion of the nucleic acid strand. Duplex nucleic acids with uninterrupted base-pairing, but with nicks in one or both strands are within the definition of a continuous duplex.

The term “duplex” refers to the state of nucleic acids in which the base portions of the nucleotides on one strand are bound through hydrogen bonding their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

The term “template” refers to a strand of nucleic acid on which a complimentary copy is built from nucleoside triphosphates through the activity of a template-dependent nucleic acid polymerase. Within a duplex the template strand is, by convention, depicted and described as the “bottom” strand. Similarly, the non-template strand is often depicted and described as the “top” strand.

As applied to polynucleotides, the term “substantial identity” denotes a characteristic of a polynucleotide sequence, wherein the polynucleotide comprises a sequence that has at least 85 percent sequence identity, in some embodiments the polynucleotide has at least 90 to 95 percent sequence identity, in specific embodiments the polynucleotide has at least 99 percent sequence identity as compared to a reference sequence over a comparison window of at least 20 nucleotide positions, in some embodiments over a window of at least 25-50 nucleotides, wherein the percentage of sequence identity is calculated by comparing the reference sequence to the polynucleotide sequence, which may include deletions or additions which total 20 percent or less of the reference sequence over the window of comparison. The reference sequence may be a subset of a larger sequence, for example, as a splice variant of the full-length sequences.

The term “label” as used herein refers to any atom or molecule that can be used to provide a detectable (in some embodiments quantifiable) effect, and that can be attached to a nucleic acid or protein. Labels include but are not limited to dyes; radiolabels such as ³²P binding moieties such as biotin; happens such as digoxigenin; luminogenic, phosphorescent or fluorogenic moieties; mass tags; and fluorescent dyes alone or in combination with moieties that can suppress (“quench”) or shift emission spectra by fluorescence resonance energy transfer (FRET).

Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

As used herein, the terms “solid support” or “support” refer to any material that provides a substrate structure to which another material can be attached. A support or substrate may be, but need not be, solid. Support materials include smooth solid supports (e.g., smooth metal, glass, quartz, plastic, silicon, wafers, carbon (e.g., diamond), and ceramic surfaces, etc.), as well as textured and porous materials. Solid supports need not be flat. Supports include any type of shape, including spherical shapes (e.g., beads). Support materials also include, but are not limited to, gels, hydrogels, aerogels, rubbers, polymers, and other porous and/or non-rigid materials.

As used herein, the terms “bead” and “particle” are used interchangeably, and refer to a small support, typically a solid support, that is capable of moving about when in a solution (e.g., it has dimensions smaller than those of the enclosure or container in which the solution resides). In some embodiments, beads may settle out of a solution when the solution is not mixed (e.g., by shaking, thermal mixing, vortexing), while in other embodiments, beads may be suspended in solution in a colloidal fashion. In some embodiments, beads are completely or partially spherical or cylindrical. However, beads are not limited to any particular three-dimensional shape. In some embodiments, beads or particles may be paramagnetic. For example, in some embodiments, beads and particles comprise a magnetic material, e.g., ferrous oxide. A bead or particle is not limited to any particular size, and in a preparation comprising a plurality of particles, the particles may be essentially uniform in size (e.g., in diameter) or may be a mixture of different sizes. In some embodiments, beads comprise or consist of nanoparticles, such as e.g., nanoparticle beads between 5 and 20 nm average diameter.

Materials attached to a solid support may be attached to any portion of the solid support (e.g., may be attached to an interior portion of a porous solid support material, or to an exterior portion, or to a flat portion on an otherwise non-flat support, or vice versa). In preferred embodiments of the technology, biological molecules such as nucleic acid or protein molecules are attached to solid supports. A biological material is “attached” to a solid support when it is affixed to the solid support through chemical or physical interaction. In some embodiments, attachment is through a covalent bond. However, attachments need not be covalent and need not be permanent. In some embodiments, an attachment may be undone or disassociated by a change in condition, e.g., by temperature, ionic change, addition or removal of a chelating agent, or other changes in the solution conditions to which the surface and bound molecule are exposed.

In some embodiments, materials are attached to a first support and are localized to the surface of a second support. For example, in some embodiments, materials that comprise a ferrous or magnetic particle may be magnetically localized to a surface or a region of a surface, such as a planar surface of a slide or well.

In some embodiments, a target molecule, e.g., a biological material, is attached to a solid support through a “spacer molecule” or “linker group.” Such spacer molecules are molecules that have a first portion that attaches to the biological material and a second portion that attaches to the solid support. Spacer molecules typically comprise a chain of atoms, e.g., carbon atoms, that provide additional distance between the first portion and the second portion. Thus, when attached to the solid support, the spacer molecule permits separation between the solid support and the biological material, but is attached to both. Examples of linkers and spacers include but are not limited to carbon chains, e.g., C3 and C6 (hexanediol), 1′,2′-dideoxyribose (dSpacer); photocleavable (PC) spacers; triethylene glycol (TEG); and hexa-ethylene glycol spacers (Integrated DNA Technologies, Inc.).

As used herein, the terms “array” and “microarray” refer a surface or vessel comprising a plurality of pre-defined loci that are addressable for analysis of the locus, e.g., to determine a result of an assay. Analysis at a locus in an array is not limited to any particular type of analysis and includes, e.g., analysis for detection of an atom, molecule, chemical reaction, light or fluorescence emission, suppression, or alteration (e.g., in intensity or wavelength) indicative of a result at that locus. Examples of pre-defined loci include a grid or any other pattern, wherein the locus to be analyzed is determined by its known position in the array pattern. Microarrays, for example, are described generally in Schena, “Microarray Biochip Technology,” Eaton Publishing, Natick, M A, 2000. Examples of arrays include but are not limited to supports with a plurality of molecules non-randomly bound to the surface (e.g., in a grid or other regular pattern) and vessels comprising a plurality of defined reaction loci (e.g., wells) in which molecules or signal-generating reactions may be detected. In some embodiments, an array comprises a patterned distribution of wells that receive beads, e.g., as described above for the SIMOA technology. See also U.S. Pat. Nos. 9,057,730; 9,556,429; 9,481,883; and 9,376,677, each of which is incorporated herein by reference in its entirety, for all purposes.

As used herein, the terms “dispersed” and “dispersal” as used in reference to loci or sites, e.g., on a support or surface, refers to a collection of loci or sites that are distributed or scattered on or about the surface, wherein at least some of the loci are sufficiently separated from other loci that they are individually detectable or resolvable, one from another, e.g., by a detector such as a microscope. Dispersed loci may be in an ordered array, or they may be in an irregular distribution or dispersal, as described below.

As used herein, the term “irregular” as used in reference to a dispersal or distribution of loci or sites, e.g., on a solid support or surface, refers to distribution of loci on or in a surface in a non-arrayed manner. For example, molecules may be irregularly dispersed on a surface by application of a solution of a particular concentration that provides a desired approximate average distance between the molecules on the surface, but at sites that are not pre-defined by or addressable any pattern on the surface or by the means of applying the solution (e.g., inkjet printing). In such embodiments, analysis of the surface may comprise finding the locus of a molecule by detection of a signal wherever it may appear (e.g., scanning a whole surface to detect fluorescence anywhere on the surface). This contrasts to locating a signal by analysis of a surface or vessel only at predetermined loci (e.g., points in a grid array), to determine how much (or what type of) signal appears at each locus in the grid.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717; 5,985,557; 5,994,069; 6,001,567; 6,090,543; and U.S. Pat. No. 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle amplification (e.g., U.S. Pat. Nos. 6,210,884, 6,183,960 and 6,235,502, herein incorporated by reference in their entireties); the variation of rolling circle amplification called “RAM amplification” (see, e.g., U.S. Pat. No. 5,942,391, incorporated herein by reference in its entirety; NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Barany Proc. Natl. Acad. Sci USA 88, 189-93 (1991)); and sandwich hybridization methods (e.g., U.S. Pat. No. 5,288,609, herein incorporated by reference in its entirety).

As used herein, the terms “digital PCR,” “single molecule PCR” and “single molecule amplification” refer to PCR and other nucleic acid amplification methods that are configured to provide amplification product or signal from a single starting molecule. Typically, samples are divided, e.g., by serial dilution or by partition into small enough portions (e.g., in microchambers or in emulsions) such that each portion or dilution has, on average as assessed according to Poisson distribution, no more than a single copy of the target nucleic acid. Methods of single molecule PCR are described, e.g., in U.S. Pat. No. 6,143,496, which relates to a method comprising dividing a sample into multiple chambers such that at least one chamber has at least one target, and amplifying the target to determine how many chambers had a target molecule; U.S. Pat. No. 6,391,559; which relates to an assembly for containing and portioning fluid; and U.S. Pat. No. 7,459,315, which relates to a method of dividing a sample into an assembly with sample chambers where the samples are partitioned by surface affinity to the chambers, then sealing the chambers with a curable “displacing fluid.” See also U.S. Pat. Nos. 6,440,706 and 6,753,147, and Vogelstein, et al., Proc. Natl. Acad. Sci. USA Vol. 96, pp. 9236-9241, August 1999. See also US 20080254474, describing a combination of digital PCR combined with methylation detection.

The term “sequencing”, as used herein, is used in a broad sense and may refer to any technique known in the art that allows the order of at least some consecutive nucleotides in at least part of a nucleic acid to be identified, including without limitation at least part of an extension product or a vector insert. In some embodiments, sequencing allows the distinguishing of sequence differences between different target sequences. Exemplary sequencing techniques include targeted sequencing, single molecule real-time sequencing, electron microscopy-based sequencing, transistor-mediated sequencing, direct sequencing, random shotgun sequencing, Sanger dideoxy termination sequencing, targeted sequencing, exon sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, ion semiconductor sequencing, nanoball sequencing, nanopore sequencing, 454 sequencing, Solexa Genome Analyzer sequencing, miSeq (Illumina), HiSeq 2000 (Illumina), HiSeq 2500 (Illumina), Illumina Genome Analyzer (Illumina), Ion Torrent PGM™ (Life Technologies), MinION™ (Oxford Nanopore Technologies), real-time SMRT™ technology (Pacific Biosciences), the Probe-Anchor Ligation (cPAL™) (Complete Genomics/BGI), SOLiD® sequencing, MS-PET sequencing, mass spectrometry, and a combination thereof. In some embodiments, sequencing comprises detecting the sequencing product using an instrument, for example but not limited to an ABI PRISM® 377 DNA Sequencer, an ABI PRISM® 310, 3100, 3100-Avant, 3730, or 3730xl Genetic Analyzer, an ABI PRISM® 3700 DNA Analyzer, or an Applied Biosystems SOLiD™ System (all from Applied Biosystems), a Genome Sequencer 20 System (Roche Applied Science), or a mass spectrometer. In certain embodiments, sequencing comprises emulsion PCR. In certain embodiments, sequencing comprises a high throughput sequencing technique, for example but not limited to, massively parallel signature sequencing (MPSS).

As used herein, the terms “digital sequencing,” “single-molecule sequencing,” and “next generation sequencing (NGS)” are used interchangeably and refer to determining the nucleotide sequence of individual nucleic acid molecules. Systems for individual molecule sequencing include but are not limited to the 454 FLX™ or 454 TITANIUM™ (Roche), the SOLEXA™/Illumina Genome Analyzer (Illumina), the HELISCOPE™ Single Molecule Sequencer (Helicos Biosciences), and the SOLID™ DNA Sequencer (Life Technologies/Applied Biosystems) instruments), as well as other platforms still under development by companies such as Intelligent Biosystems and Pacific Biosystems. See also U.S. Pat. No. 7,888,017, entitled “Non-invasive fetal genetic screening by digital analysis,” relating to digital analysis of maternal and fetal DNA, e.g., cfDNA.

As used herein, the terms “crowding agent” and “volume excluder,” as used in reference to a component of a fluid reaction mixture, are used interchangeably and refer to compounds, generally polymeric compounds, that reduce available fluid volume in a reaction mixture, thereby increasing the effective concentration of reactant macromolecules (e.g., nucleic acids, enzymes, etc.) Crowding reagents include, e.g., glycerol, ethylene glycol, polyethylene glycol, ficoll, serum albumin, casein, and dextran.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “MIP” as used herein, refers to a molecular inversion probe (or a circular capture probe). Molecular inversion probes (or circular capture probes) are nucleic acid molecules that comprise a pair of unique polynucleotide arms that hybridize to a target nucleic acid to form a nick or gap and a polynucleotide linker (e.g., a universal backbone linker). In some embodiments, the unique polynucleotide arms hybridize to a target strand immediately adjacent to each other to form a ligatable nick (generally termed “padlock probes”) while in some embodiments, the hybridized MIP must be further modified (e.g., by polymerase extension, base excision, and/or flap cleavage) to form a ligatable nick. Ligation of a MIP probe to form a circular nucleic acid is typically indicative of the presence of the complementary target strand. In some embodiments, MIPs comprise one or more unique molecular tags (or unique molecular identifiers). See, for example, FIG. 1. In some embodiments, a MIP may comprise more than one unique molecular tags, such as, two unique molecular tags, three unique molecular tags, or more. In some embodiments, the unique polynucleotide arms in each MIP are located at the 5′ and 3′ ends of the MIP, while the unique molecular tag(s) and the polynucleotide linker are located internal to the 5′ and 3′ ends of the MIP. In some embodiments, the MIP is a 5′ phosphorylated single-stranded nucleic acid (e.g., DNA) molecule. See, for example, WO 2017/020023, filed Jul. 29, 2016, and WO 2017/020024, filed Jul. 29, 2016, each of which is incorporated by reference herein for all purposes.

As used herein, the terms “circular nucleic acid” and “circularized nucleic acid” as used, for example, in reference to probe nucleic acids, refers to nucleic acid strands that are joined at the ends, e.g., by ligation, to form a continuous circular strand of nucleic acid.

The unique molecular tag may be any tag that is detectable and can be incorporated into or attached to a nucleic acid (e.g., a polynucleotide) and allows detection and/or identification of nucleic acids that comprise the tag. In some embodiments the tag is incorporated into or attached to a nucleic acid during sequencing (e.g., by a polymerase). Non-limiting examples of tags include nucleic acid tags, nucleic acid indexes or barcodes, radiolabels (e.g., isotopes), metallic labels, fluorescent labels, chemiluminescent labels, phosphorescent labels, fluorophore quenchers, dyes, proteins (e.g., enzymes, antibodies or parts thereof, linkers, members of a binding pair), the like or combinations thereof. In some embodiments, particularly sequencing embodiments, the tag (e.g., a molecular tag) is a unique, known and/or identifiable sequence of nucleotides or nucleotide analogues (e.g., nucleotides comprising a nucleic acid analogue, a sugar and one to three phosphate groups). In some embodiments, tags are six or more contiguous nucleotides. A multitude of fluorophore-based tags are available with a variety of different excitation and emission spectra. Any suitable type and/or number of fluorophores can be used as a tag. In some embodiments 1 or more, 2 or more, 3 or more, 4 or more, 5 or more, 6 or more, 7 or more, 8 or more, 9 or more, 10 or more, 20 or more, 30 or more, 50 or more, 100 or more, 500 or more, 1000 or more, 10,000 or more, 100,000 or more different tags are utilized in a method described herein (e.g., a nucleic acid detection and/or sequencing method). In some embodiments, one or two types of tags (e.g., different fluorescent labels) are linked to each nucleic acid in a library. In some embodiments, chromosome-specific tags are used to make chromosomal counting faster or more efficient. Detection and/or quantification of a tag can be performed by a suitable method, machine or apparatus, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In the MIPs, the unique polynucleotide arms are designed to hybridize immediately upstream and downstream of a specific target sequence (or site) in a nucleic acid target. In some embodiments, hybridization of a MIP to a target sequence produces a ligatable nick without a gap, i.e., the two arms of the MIP hybridize to contiguous sequences in the target strand such that no overlap or gap is formed upon hybridization. Such zero-gap MIPs are generally termed “padlock” probes. See, e.g., M. Nilsson, et al. “Padlock probes: circularizing oligonucleotides for localized DNA detection”. Science. 265 (5181): 2085-2088 (1994); J. Banér, et al., Nucleic Acids Res., 26 (22):5073-5078 (1998). In other embodiments the hybridized MIP/target nucleic acid complex requires modification to produce a ligatable nick. For example, in some embodiments, hybridization leaves a gap that is filled, e.g., by polymerase extending a 3′ end of the MIP, prior to ligation, while in other embodiments, hybridization forms an overlapping flap structure that must be modified, e.g., by a flap endonuclease or a 3′ exonuclease, to produce a ligatable nick. In some embodiments, MIPS comprise unique molecular tags are short nucleotide sequences that are randomly generated. In some embodiments, the unique molecular tags do not hybridize to any sequence or site located on a genomic nucleic acid fragment or in a genomic nucleic acid sample. In some embodiments, the polynucleotide linker (or the backbone linker) in the MIPs is universal in all the MIPs used in embodiments of this disclosure.

In some embodiments, the MIPs are introduced to nucleic acid fragments to perform capture of target sequences or sites (or control sequences or sites) located on a nucleic acid sample. As described in greater detail herein, after capture of the target sequence (e.g., locus) of interest, the captured target may be subjected to enzymatic gap-filling and ligation steps, such that a copy of the target sequence is incorporated into a circle-like structure. In some embodiments, nucleic acid analogs, e.g., containing labels, haptens, etc., may be incorporated in the filled section, for use, e.g., in downstream detection, purification, or other processing steps. Capture efficiency of the MIP to the target sequence on the nucleic acid fragment can, in some embodiments, be improved by lengthening the hybridization and gap-filling incubation periods. (See, e.g., Turner E H, et al., Nat Methods. 2009 Apr. 6:1-2.).

MIP technology may be used to detect or amplify particular nucleic acid sequences in complex mixtures. One of the advantages of using the MIP technology is in its capacity for a high degree of multiplexing, which allows thousands of target sequences to be captured in a single reaction containing thousands of MIPs. Various aspects of MIP technology are described in, for example, Hardenbol et al., “Multiplexed genotyping with sequence-tagged molecular inversion probes,” Nature Biotechnology, 21(6): 673-678 (2003); Hardenbol et al., “Highly multiplexed molecular inversion probe genotyping: Over 10,000 targeted SNPs genotyped in a single tube assay,” Genome Research, 15: 269-275 (2005); Burmester et al., “DMET microarray technology for pharmacogenomics-based personalized medicine,” Methods in Molecular Biology, 632: 99-124 (2010); Sissung et al., “Clinical pharmacology and pharmacogenetics in a genomics era: the DMET platform,” Pharmacogenomics, 11(1): 89-103 (2010); Deeken, “The Affymetrix DMET platform and pharmacogenetics in drug development,” Current Opinion in Molecular Therapeutics, 11(3): 260-268 (2009); Wang et al., “High quality copy number and genotype data from FFPE samples using Molecular Inversion Probe (MIP) microarrays,” BMC Medical Genomics, 2:8 (2009); Wang et al., “Analysis of molecular inversion probe performance for allele copy number determination,” Genome Biology, 8(11): R246 (2007); Ji et al., “Molecular inversion probe analysis of gene copy alternations reveals distinct categories of colorectal carcinoma,” Cancer Research, 66(16): 7910-7919 (2006); and Wang et al., “Allele quantification using molecular inversion probes (MIP),” Nucleic Acids Research, 33(21): e183 (2005), each of which is hereby incorporated by reference in its entirety for all purposes. See also in U.S. Pat. Nos. 6,858,412; 5,817,921; 6,558,928; 7,320,860; 7,351,528; 5,866,337; 6,027,889 and 6,852,487, each of which is hereby incorporated by reference in its entirety for all purposes.

The term “capture” or “capturing”, as used herein, refers to the binding or hybridization reaction between a capture probe, such as a molecular inversion probe, and its corresponding targeting site. In some embodiments, upon capturing, a circular replicon or a MIP replicon is produced or formed. In some embodiments, the targeting site is a deletion (e.g., partial or full deletion of one or more exons). As used in reference to other oligonucleotides, e.g., “capture oligonucleotide” the term refers to a binding or hybridization reaction between the capture oligonucleotide and a nucleic acid to be captured, e.g., to be immobilized, removed from solution, or otherwise be manipulated by hybridization to the capture oligonucleotide.

The term “MIP replicon” or “circular replicon”, as used herein, refers to a circular nucleic acid molecule generated via a capturing reaction (e.g., a binding or hybridization reaction between a MIP and its targeted sequence). In some embodiments, the MIP replicon is a single-stranded circular nucleic acid molecule. In some embodiments, a targeting MIP captures or hybridizes to a target sequence or site. After the capturing reaction or hybridization, in some embodiments, a ligation reaction mixture is introduced to ligate the nick formed by hybridization of the two targeting polynucleotide arms to form single-stranded circular nucleotide molecules, i.e., a targeting MIP replicon, while in some embodiments, hybridization of the MIP leaves a gap, and a ligation/extension mixture is introduced to extend and ligate the gap region between the two targeting polynucleotide arms to form a targeting MIP replicon. In some embodiments, a control MIP captures or hybridizes to a control sequence or site. After the capturing reaction or hybridization, a ligation reaction mixture is introduced to ligate the nick formed by hybridization of the two control polynucleotide arms, or a ligation/extension mixture is introduced to extend and ligate the gap region between the two control polynucleotide arms to form single-stranded circular nucleotide molecules, i.e., a control MIP replicon. MIP replicons may be amplified through a polymerase chain reaction (PCR) to produce a plurality of targeting MIP amplicons, which are double-stranded nucleic acid molecules. MIP replicons find particular application in rolling circle amplification, or RCA. RCA is an isothermal nucleic acid amplification technique where a DNA polymerase continuously adds single nucleotides to a primer annealed to a circular template, which results in a long concatemer of single stranded DNA that contains tens to hundreds to thousands of tandem repeats (complementary to the circular template). See, e.g., M. Ali, et al. “Rolling circle amplification: a versatile tool for chemical biology, materials science and medicine”. Chemical Society Reviews. 43 (10): 3324-3341, which is incorporated herein by reference in its entirety, for all purposes. See also WO 2015/083002, which is incorporated herein by reference in its entirety, for all purposes. Polymerases typically used in RCA for DNA amplification are Phi29, Bst, and Vent exo-DNA polymerases, with Phi29 DNA polymerase being preferred in view of its superior processivity and strand displacement ability

The term “amplicon”, as used herein, refers to a nucleic acid generated via amplification reaction (e.g., a PCR reaction). In some embodiments, the amplicon is a single-stranded nucleic acid molecule. In some embodiments, the amplicon is a double-stranded nucleic acid molecule. In some embodiments, a targeting MIP replicon is amplified using conventional techniques to produce a plurality of targeting MIP amplicons, which are double-stranded nucleotide molecules. In some embodiments, a control MIP replicon is amplified using conventional techniques to produce a plurality of control MIP amplicons, which are double-stranded nucleotide molecules.

The term “signal” as used herein refers to any detectable effect, such as would be caused or provided by a label or by action or accumulation of a component or product in an assay reaction.

As used herein, the term “detector” refers to a system or component of a system, e.g., an instrument (e.g. a camera, fluorimeter, charge-coupled device, scintillation counter, solid state nanopore device, etc..) or a reactive medium (X-ray or camera film, pH indicator, etc.), that can convey to a user or to another component of a system (e.g., a computer or controller) the presence of a signal or effect. A detector is not limited to a particular type of signal detected, and can be a photometric or spectrophotometric system, which can detect ultraviolet, visible or infrared light, including fluorescence or chemiluminescence; a radiation detection system; a charge detection system; a system for detection of an electronic signal, e.g., a current or charge perturbation; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

The term “detection” as used herein refers to quantitatively or qualitatively identifying an analyte (e.g., DNA, RNA or a protein), e.g., within a sample. The term “detection assay” as used herein refers to a kit, test, or procedure performed for the purpose of detecting an analyte within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, nucleic acid ligation, antigen-antibody binding, interaction of a primary antibody with a secondary antibody, and/or conformational change in a nucleic acid (e.g., an oligonucleotide) or polypeptide (e.g., a protein or small peptide).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a sub portion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520I of the Federal Food, Drug, and Cosmetic Act, but are not limited thereto. Indeed, any delivery system comprising two or more separate containers that each contains a sub portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### BRIEF DESCRIPTION OF THE DRAWINGS

FIG. 1 shows an empirical distribution of the fetal fraction in cfDNA extracted from maternal plasma samples.

FIG. 2 schematically illustrates the reasoning behind the presence of fetally enriched/depleted genomic regions. A. Possible biological mechanism underlying the presence of genomic regions that positively correlate with fetal fraction. B. Possible biological mechanism underlying the presence of genomic regions that negatively correlate with fetal fraction

FIG. 3 is a flowchart illustrating a method of estimating fetal fraction.

FIG. 4 is a flowchart illustrating a method of providing an assay for estimating fetal fraction.

FIG. 5 shows an embodiment of a system for estimating fetal fraction according to the present disclosure.

FIG. 6 provides a schematic diagram of a molecular inversion probe (MIP) for chromosome-specific recognition, suitable for use in massively multiplexed capture assays.

FIG. 7 provides a schematic diagram of an embodiment of multiplexed chromosome-specific rolling circle amplification.

FIG. 8 provides a schematic diagram of an embodiment of multiplexed chromosome-specific rolling circle amplification using molecular beacon probes for detection.

FIG. 9 is a flowchart illustrating a method of detecting fetal aneuploidies.

FIG. 10 shows the mean read count per 1 kb site as a function of the fetal fraction estimated using chromosome Y count data, for each of the sites that were identified as negatively or positively correlated with fetal fraction in a sequencing data set comprising approximately 12 k male fetus samples.

FIG. 11 shows the fetal fraction gain for each of a set of sites that were identified as described herein as being correlated with fetal fraction. Each point represents a set of sites that is associated with a Ridge regression gain within a range corresponding to 1 percentile of the distribution of Ridge regression gains estimated across all sites.

FIG. 12 shows the enrichment of DNAse H Site (from the ENCODE database) for each of a plurality of tissues, in sites that were identified as negatively correlated with fetal fraction, positively correlated with fetal fraction, or not significantly correlated with fetal fraction.

FIG. 14 shows the enrichment of DNAse H Site (from the ENCODE database) in placenta, in sites that were identified as correlated with fetal fraction.

FIG. 15 shows the distribution of fractional counts per site for pairs of samples that have different (known) fetal fractions, for sites that were identified as negative predictors of fetal fraction (top plot), and sites that were identified as positive predictors of fetal fraction (bottom plot).

FIG. 16 shows the fractional counts for selected sites for a plurality of groups of samples associated with different mean fetal fractions.

FIG. 17 shows the mean fractional counts for a set of x combined sites as a function of fetal fraction, for each of 50 sets of samples associated with different fetal fractions (A), as well as the coefficient of variation for this signal (B).

FIG. 18 shows the mean fractional counts for combined sets of sites identified by hierarchical clustering of sites using the Ridge gain as a clustering feature (A), as well as the same data for the two largest clusters only (B).

FIG. 19 shows the maternal and fetal enrichment factors for 5000 most significant and highest-effect-size genomic sites identified in a discovery process described herein. Maternal enrichment factor vs. fetal enrichment factor; the dashed line is the identity line shown for guiding the eye. Color scale encodes number of loci that have a specific combination of maternal and fetal enrichment factors.

FIG. 20 shows the distribution of the enrichment ratio (defined as fetal/maternal enrichment) for the sites of FIG. 19. The vertical dashed line denotes the “neutral” decision boundary (non-informative loci with enrichment ratio=1).

FIG. 21 shows a study of the point sensitivity (power) of calling fetal fraction using informative sites. The results are shown vs. the fetal fraction, and for different numbers of targeted sites (300, 500, 1000, 2000). All targeted sites were assumed to have the same fetal/maternal enrichment ratio (columns: 0.8, 0.9). Performance was quantified using the absolute relative error metric, at different relative error levels: 10%, 25%, 50% (rows). Population abundance of fetal fractions was simulated by drawing the samples' fetal fractions from the empirical distribution in FIG. 1.

FIG. 22 shows a study of the cumulative sensitivity of calling fetal fraction using informative sites. The same scenarios and the same performance metrics as in FIG. 21 were considered here. For a given fetal fraction level, cumulative sensitivity was defined as the sensitivity of calling fetal fraction for samples with fetal fractions equal to or higher than the given fetal fraction level.

FIG. 23 is a flowchart illustrating a protocol for the generation of targeted data (using molecular inversion probes) to validate the estimation of fetal fraction using targeted molecular counts.

FIG. 24 shows the results of an experiment using whole-genome amplified cfDNA as the input for hybridization capture and ligation of MIPs. The plot compares fetal fraction estimates from molecular counts ratios for chromosome Y sequences, and fetal fraction estimated using a control method.

FIG. 25 shows results for the experiment on FIG. 24, revealing increasing depletion of molecular counts at selected genomic loci with increasing fetal fraction. The plot shows the count ratios for 100 top-performing sites as a function of the fetal fraction estimated using a control method.

FIG. 26 compares the results obtained in the experiment of FIGS. 24-25 (targeting using MIPs) and the results obtained in the experiment of FIGS. 19-20 By grouping the designed MIPs according to the response properties of their hosting sites, stronger dependence of the signal (read counts for grouped MIPs) on the fetal fraction was observed for all selected MIPs.

FIG. 27 shows a strong and clean dependence of signal depletion on the fetal fraction can be observed when using pooled cfDNA samples instead of whole genome amplified samples. The plot shows the results for pooled samples containing 10 nanograms (LFF_10_S1), 5 nanograms (LFF_5_S1), and 1 nanograms (LFF_1_S1) at the 5% fetal fraction, and pooled samples containing 10 nanograms (HFF_10_S1), 5 nanograms (HFF_5_S1), and 1 nanograms (HFF_1_S1) at the 16% fetal fraction.

### DETAILED DESCRIPTION OF THE INVENTION

The present invention provides a solution to the problem of estimating fetal fraction, particularly in the context of non-invasive prenatal diagnostic tests that do not rely on whole genome/exome sequencing. Thus, in some embodiments, the technologies provided herein provide means to estimate fetal fraction using economical methods for testing samples in a manner that counts the number of copies of a specific nucleic acid or protein in a sample or portion of a sample in a digital manner, i.e., by detecting individual copies of the molecules, without use of a sequencing step (e.g., a digital or “next gen” sequencing step).

The invention makes use of regions of the genome whose representation in mixed maternal-fetal cfDNA samples correlates with the fetal fraction in said samples. These regions are referred to herein as “informative regions”. Probes that specifically target these regions, such as e.g., capture probes such as molecular inversion probes, are referred to herein as “informative probes”. By contrast, regions of the genome whose representation in mixed maternal-fetal cfDNA samples does not correlate with the fetal fraction in said samples may be referred to as “uninformative regions”. Probes that specifically target these regions, such as e.g., capture probes such as molecular inversion probes, are referred to herein as “uninformative probes”. Uninformative probes may be included in an assay for example for purposes other than the estimation of the fetal fraction, such as e.g., detection of gene dosage variations or aneuploidy.

The discovery that circulating free DNA in pregnant women comprised DNA derived from the developing fetus spurred the non-invasive prenatal testing industry for the last decade. The origin and defining characteristics of fetal cfDNA have been extensively researched. Interestingly, although the entire fetal genome can be isolated from circulating blood of a pregnant woman, studies suggest there is not a uniform distribution of the fetal genome in the cfDNA. This implies that some regions of the fetal genome may be either in excess or depleted. One theory on why this occurs is the mechanism by which cellular DNA becomes fragmented and transported into the bloodstream. The idea is that a cell undergoing cell death (through a variety of processes including apoptosis, necrosis, autophagy, etc.) initiates a complex process by which the genomic DNA is degraded enzymatically. This degradation processes are influenced by whether these enzymatic complexes have access to the genome. Therefore, DNA undergoing active transcription may be more accessible to the degradation machinery. Thus, regions undergoing transcription may be rapidly degraded to very small fragments or nucleotides, which are less likely to be observed in the blood. The converse would also be true: regions of the genome not being actively transcribed would have a restrictive environment of chromatin that would slow or stop degradation. Thus, regions of the genome that are not undergoing active transcription may be more likely to be observed in the blood.

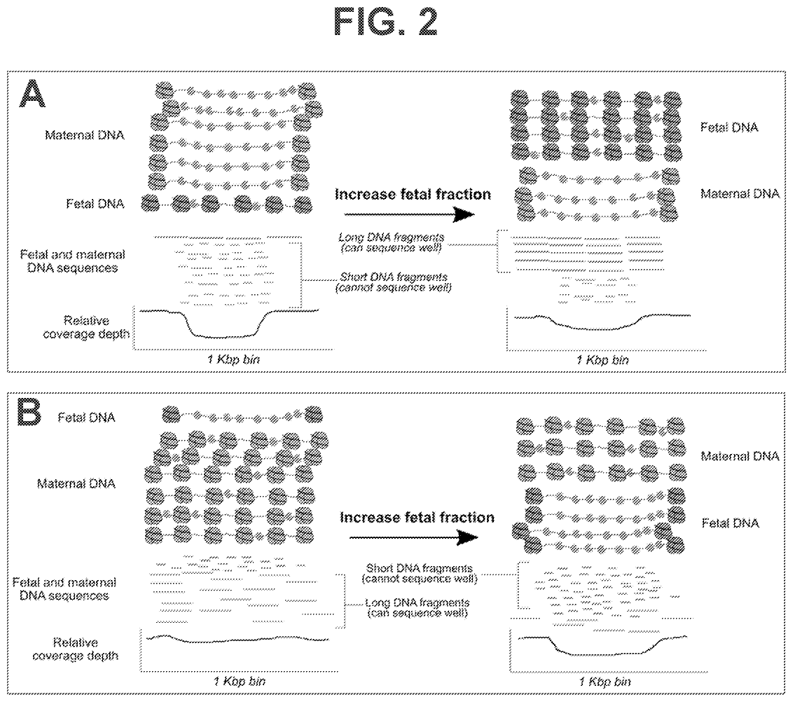

This is the fundamental concept that underlines this invention and is illustrated on FIG. 2. Both fetal cfDNA and maternal cfDNA are circulating together but are derived from the cell death processes of at least two different cell types. It has been suggested that fetal cfDNA obtained from pregnant woman may be derived from only one cell type in the placenta, the trophoblast. By contrast, the maternally-derived cfDNA in pregnant women's blood may be derived from a range of maternal tissues. The expression profile of the fetal trophoblast and the cell types that derive the maternal cfDNA are likely to differ. As a consequence, the pattern of accessibility to degradation in the genomes of these cell types may differ, and so would the relative representation of genomic regions in cell free DNA. For example, as illustrated in FIG. 2B, a region of the genome that is highly expressed in fetal trophoblasts (illustrated as open chromatin in fetal DNA on FIG. 2B) would be depleted from the fetal cfDNA (illustrated on FIG. 2B as producing mostly short, degraded DNA fragments that are unlikely to be identified efficiently in a cfDNA sample). The same region of the genome in maternal cells may contain genes that are expressed at very low levels, bound to closed chromatin, and resistant to degradation (illustrated as closed chromatin on FIG. 2B, producing mostly longer undegraded fragments that are likely to be identified efficiently in a cfDNA sample). The signal, such as read counts via sequencing or other molecular counting assay, from this site would appear depleted with increasing fetal fraction concentrations (compare FIG. 2B, left and FIG. 2B, right). In other words, increasing fetal fraction leads to an increased proportion of degraded fragments derived from the site under investigation (which may contain genes that are more likely to be expressed in fetal tissue than in maternal tissue). These degraded fragments are not efficiently detected, and as such the counts of molecules that can be attributed to such genomic sites decreases as fetal fraction increases. Thus, molecular counts from such sites may be expected to be negatively associated with fetal fraction. FIG. 2A illustrates the reverse situation. A region of the genome that contain genes that are not expressed in fetal trophoblasts (illustrated as closed chromatin in fetal DNA on FIG. 2A) would be well represented in the fetal cfDNA (illustrated on FIG. 2A as producing mostly non-degraded DNA fragments that are likely to be identified efficiently in a cfDNA sample). The same region of the genome in maternal cells may contain genes that are expressed at higher levels and subject to degradation (illustrated as open chromatin on FIG. 2A, producing mostly degraded fragments that are unlikely to be identified efficiently in a cfDNA sample). The signal, such as read counts via sequencing, from this site would appear enriched with increasing fetal fraction (compare FIG. 2A, left and FIG. 2A, right). In other words, increasing fetal fraction leads to an increased proportion of undegraded fragments derived from the site under investigation. These fragments are efficiently detected, and as such the counts of molecules that can be attributed to such genomic sites increases as fetal fraction increases. Thus, molecular counts from such sites may be expected to be positively associated with fetal fraction. Although not depicted on FIG. 2, many genomic regions contain genes that are unlikely to be significantly differentially expressed in maternal and fetal tissue or have differential chromatin accessibility. The amounts of DNA derived from such regions in cfDNA would therefore not be representative of fetal fraction. These regions may be referred to as “uninformative” regions. Uninformative regions may be regions that contain genes that are either observed or not observed in both the fetal and maternal cell types from which the cfDNA is derived, such as e.g., regions comprising genes that are essential to the metabolism of all cells or regions that do not contain genes or other functional elements. Uninformative regions may also be regions that contain genes which are not consistently differentially expressed between fetal and maternal cell types from which the cfDNA is derived. For example, the region may contain genes consistently expressed or not expressed in fetal tissue, but may be inconsistently expressed in different maternal tissues that are represented in variable amounts in cell-free DNA. As another example, the region may contain genes which are inconsistently expressed in fetal tissue, such that e.g., the region may contain genes very likely to be expressed at a particular timing during pregnancy and much less likely to be expressed at another timing during pregnancy. Such a region would also not be reliably associated with fetal fraction.

Therefore, the present invention is based on the hypothesis that nonuniformity in genomic representation of the observed fetal cfDNA compared to the maternal cfDNA in specific genomic regions may provide a way to experimentally determine the percentage of fetal cfDNA in the blood from a pregnant woman. By identifying the sites with differences between molecular counts from maternal cfDNA and fetal cfDNA, development of an experimental approach for estimating the fetal fraction of a single blood sample by targeted molecular counting of cfDNA molecules derived from such sites can be envisioned. Note that this invention depends on differential representation of cfDNA fragments and does not depend on gene expression levels, characterization of functional characteristics of regions, measurements of chromatin structure or accessibility. Exemplary informative sites are illustrated on FIG. 2 as 1 kb regions of the genome. Diving the genome in such regions was identified as a convenient way to roughly locate informative sites. However, in practice the informative regions are not expected to align with arbitrarily defined boundaries and may vary in size significantly. Thus, the inventors have identified that reliable estimation of fetal fraction using molecular counts from specific regions could be enabled by precisely identifying sub-regions within arbitrarily defined boundaries that produced a significant and reliable signal.

### Methods of Estimating Fetal Fraction

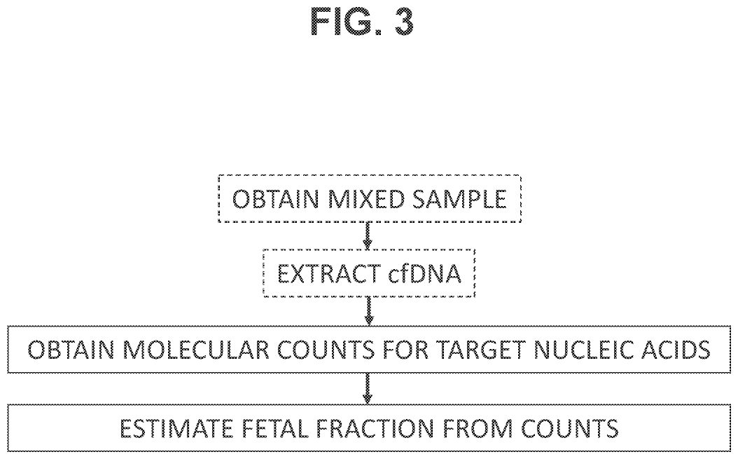

FIG. 3 shows an embodiment of a method for estimating the fetal fraction in a mixed sample comprising fetal and maternal DNA. The method comprises obtaining molecular counts for a plurality of predetermined genomic regions (target nucleic acids), the plurality of genomic regions comprising a first set of regions (also referred to herein as “informative sites), wherein regions in the first set of regions are chosen such that molecular counts from mixed samples comprising fetal and maternal DNA for these regions are significantly associated with fetal fraction according to a statistical model, and estimating the fetal fraction in the mixed sample using at least the molecular counts for the first set of regions and a statistical model that uses the molecular counts or variables derived therefrom as predictor variables, and the fetal fraction as predictor variable. The plurality of predetermined genomic regions may further comprise a second set of regions (also referred to herein as “uninformative sites”), wherein regions in the second set of regions are chosen such that molecular counts from mixed samples comprising fetal and maternal DNA for these regions are not significantly associated with fetal fraction according to a statistical model. In some embodiments, the regions in the second set of regions may be chosen as autosomal regions that are associated with counts that only exhibit stochastic sampling variability in a training data set. In other embodiments, the regions in the second set of regions may be associated with an enrichment factor (ratio of the maternal and fetal enrichment factor) that is close to 1, or not significantly different from 1. The step of estimating the fetal fraction in the mixed sample may use the molecular counts from the first and second sets of genomic regions, such as, for example, using a ratio of the molecular counts from the first and second sets of genomic regions. In some embodiments, the regions in the second set of regions may be used to obtain a metric derived from the molecular counts from the first set of genomic regions, that is normalised to control for assay yield. The plurality of predetermined genomic regions may further comprise a third (resp. fourth, fifth, etc.) set of regions, wherein regions in the third (resp. fourth, fifth) set of regions are chosen such that molecular counts from mixed samples comprising fetal and maternal DNA for these regions are indicative of one or more fetal abnormalities. In some embodiments, the step of obtaining molecular counts for a first set of predetermined genomic regions may comprises combining molecular counts for a plurality of target nucleic acids located within a plurality of regions within the first set of predetermined regions. The counts for the respective regions may be combined using a weighted sum. The weights used may correspond to or be derived from an enrichment factor obtained from training data. Further, the step of obtaining molecular counts for a second set of predetermined genomic regions may comprises combining molecular counts for a plurality of target nucleic acids located within a plurality of regions within the second set of predetermined regions. The method may further comprise the optional step of obtaining a mixed sample comprising fetal and maternal DNA. Typically, this may be a blood sample. The step of obtaining a sample may further comprise processing the sample, such as e.g., for storage or purification. For example, the step of obtaining a mixed sample may comprise obtaining a maternal blood sample and separating out cellular components, thereby obtaining a plasma sample. The method may further comprise the step of extracting cell free DNA from the sample. The step of obtaining molecular counts for target nucleic acids may comprise the step of selectively measuring the amount of DNA in the sample that can be attributed to the predetermined regions. Alternatively, the step of obtaining molecular counts for a plurality of predetermined genomic regions may comprise receiving or extract molecular count data for the plurality of predetermined genomic regions. Thus, the methods described herein may include exclusively in silico steps, or a combination of in silico and in vitro steps. As the skilled person understands, the step of estimating a fetal fraction from molecular counts from a plurality of predetermined regions is typically well beyond the reach of mental capabilities due to the number of individual regions and the complexity of the counts data that is typically analyzed.

In embodiments, the molecular counts for each of the plurality of predetermined genomic regions are obtained as combined (also referred to herein as cumulative) counts for any sequence that is within a predetermined region. In embodiments, the molecular counts for the first (respectively second, third, etc.) set of regions are obtained as combined counts for any sequence that is within any of the predetermined regions in the first (respectively second, third, etc.) set.

The molecular counts may have been obtained using any suitable method known in the art, such as e.g., digital counting assays, microarrays, targeted sequencing, etc. The step of obtaining molecular counts may comprise one or more preprocessing steps selected from: filtering (e.g., based on unique molecular identifiers, quality control parameters, etc.), normalization, transformation (such as e.g., transformation), adjustment for sequence characteristics (such as e.g., GC content, genomic similarity, etc.). The first set of regions may each have a size individually chosen between approximately 10 bases and approximately 100 kb, in some embodiments between approximately 100 bases and approximately 10 kb, such as around 1 kb. In a related embodiment, the regions in the first set of regions may each have a size individually chosen between approximately 1 kb and approximately 20 kb. The regions in the second set of regions may each have a size individually chosen between approximately 10 bases and approximately 100 kb. The regions in the second set of regions may each have a size individually chosen between approximately 1 kb and approximately 20 kb. The first and/or second set of genomic regions may comprise regions located on autosomal chromosomes. The first and/or second set of genomic regions may consist of regions located on autosomal chromosomes.

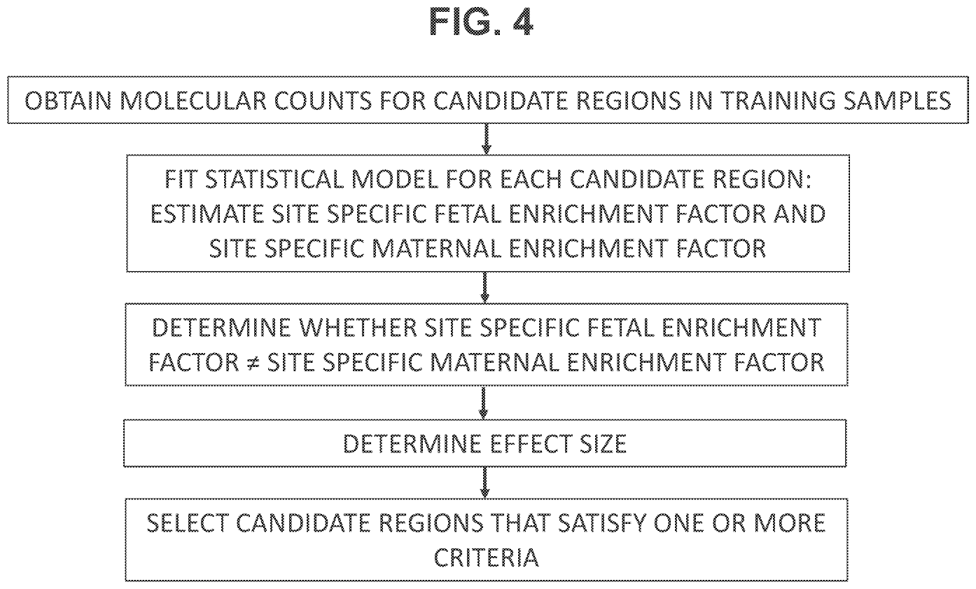

The statistical model may model the expected molecular count for a region in the genome as the product of the total number of counts obtained from a cfDNA sample from sites with known ploidy (also referred to herein as “assay yield”) and a region (or site) enrichment factor that is expressed as a weighted combination of a maternal enrichment factor (with weight equal to 1-fetal fraction) and a fetal enrichment factor (with weight equal to the fetal fraction). The expected molecular count for a region in the genome may be assumed to have any suitable distribution. For example, the expected molecular count for a region may be assumed to have a Poisson distribution, a negative binomial distribution or a normal distribution. As an example, a Poisson distribution may be particularly suitable for count data that is not expected or observed to be over dispersed. A negative binomial distribution may be useful to model count data that is expected or observed to be over dispersed. A normal distribution may be useful for count data that is observed to be approximately normal (typically after transformation). The suitability of a particular distribution to model a particular data set may be determined using any method known in the art for assessing goodness of fit. For example, methods for assessing the normality of a distribution are known. As shown on FIG. 4, the regions in the first set of regions may be selected by: (i) fitting a statistical model to molecular counts from a set of mixed samples (also referred to herein as training samples) comprising fetal and maternal DNA for a plurality of candidate regions (where the candidate regions may e.g., represent the entire genome), wherein the statistical model comprises a site-specific fetal enrichment factor and a site-specific maternal enrichment factor for each candidate region, and a fetal fraction for each sample as parameters of the model, and (ii) determining whether a candidate region significantly associated with fetal fraction according to the statistical model by comparing the site-specific fetal enrichment factor and the site-specific maternal enrichment factor estimated for the site through the fitting of the model. Fitting a model may comprise identifying a set of parameters that maximizes a log likelihood function calculated for a set of training data. The step of selecting regions in the first set of regions may further comprise determining the differential enrichment effect size. The differential enrichment effect size may be calculated as the difference (or absolute difference) between the site-specific fetal enrichment factor and the site-specific maternal enrichment factor. The step of selecting candidate regions may further comprise selecting candidate regions that satisfy one or more criteria selected from: the site-specific fetal enrichment factor being significantly different from the site-specific maternal enrichment factor (where a significant difference may be determined using a p-value threshold), and/or the differential enrichment effect size being above a predetermined threshold (where the predetermined threshold may be defined as a threshold on the effect size or as a threshold derived from the distribution of effect sizes across candidate regions, such as e.g., the effect size threshold that includes the 100, 1000, 2000, 4000, 5000, or 10,000 regions with higher effect size, the threshold that includes the 1^{st}, 2^{nd}, 5^{th}, 10^{th} percentile of the distribution of effect size, etc.), and/or the candidate region being in the set of regions that has the highest significance (such as e.g., the top 100, 1000, 2000, 4000, 5000, or 10,000 regions that have the most significant differential effect size amongst the candidate regions tested, or the 0.1, 0.5, 1 or 2% of regions tested that have the most significant differential effect size).

The step of estimating the fetal fraction may comprise using a generalized linear model that models that molecular counts for one or more regions in the first set of regions as a predictor variable and the fetal fraction as a response variable.

### Methods of Providing an Assay for Estimating Fetal Fraction

Providing an assay for estimating fetal fraction may comprise selecting candidate regions as described above and in relation to FIG. 4. A set of target nucleic acids that are located in the candidate regions may then be selected, and an assay that produces molecular counts for these targets may be designed. For example, the candidate regions may each be targeted using one or more molecular inversion probes that are designed to capture sequences located in the candidate regions. As the skilled person understands, the particular identity of the target nucleic acids may depend on the experimental platform used. The assay may then be applied to one or more test samples, each associated with a known or estimated fetal fraction. The fetal fraction estimated from the molecular counts derived from the assay (as explained above) using selections of the target nucleic acids may be compared in order to select particular target nucleic acid sequences that are associated with reliable fetal fraction estimates. Additionally, the molecular counts derived from the assay for particular target nucleic acids for samples with known fetal fraction may be used to identify target nucleic acids that are associated with low variability counts between samples with similar fetal fractions. For example, the molecular counts for a candidate target nucleic acid sequence (or candidate set of target nucleic acid sequences) may be combined in each of a plurality of groups of samples that have similar known or estimated fetal fractions, and a measure of molecular count variability within such groups may be obtained. As a specific example, the molecular counts for a candidate target nucleic acid sequence (or candidate set of target nucleic acid sequences) may be combined in each of 50 groups of samples that have known or estimated fetal fractions within contiguous ranges that span the whole range of fetal fractions observed.

### Systems

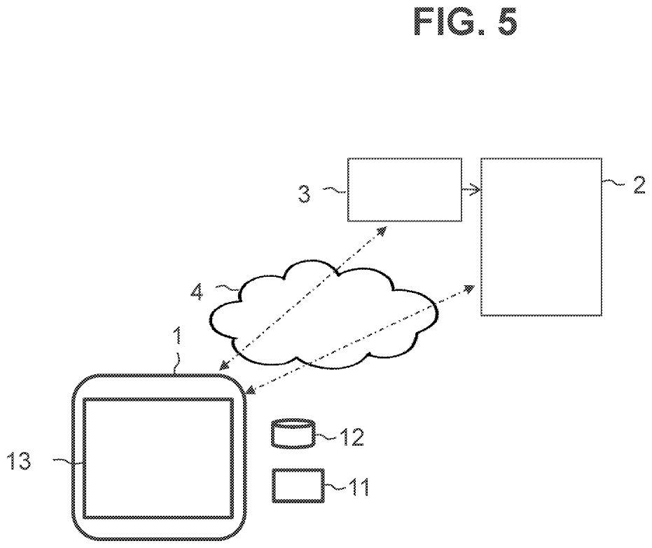

FIG. 5 shows an embodiment of a system for estimating fetal fraction according to the present disclosure. The system comprises a computing device 1, which comprises a processor 11 and computer readable memory 12. In the embodiment shown, the computing device 1 also comprises a user interface 13, which is illustrated as a screen but may include any other means of conveying information to a user such as e.g., through audible or visual signals. The computing device 1 is communicably connected, such as e.g., through a network, to molecular count acquisition means 3, and/or to one or more databases 2 storing molecular counts data. The computing device may be a smartphone, tablet, personal computer or other computing device. The computing device is configured to implement a method for estimating fetal fraction, as described herein. In alternative embodiments, the computing device 1 is configured to communicate with a remote computing device (not shown), which is itself configured to implement a method of processing images, as described herein. In such cases, the remote computing device may also be configured to send the result of the method of estimating fetal fraction to the computing device. Communication between the computing device 1 and the remote computing device may be through a wired or wireless connection, and may occur over a local or public network such as e.g., over the public internet. The image acquisition means may be in wired connection with the computing device 1, or may be able to communicate through a wireless connection, such as e.g., through WiFi, as illustrated. The connection between the computing device 1 and the image acquisition means 3 may be direct or indirect (such as e.g., through a remote computer). The molecular count acquisition means 3 are configured to acquire molecular count data for specifically targeted nucleic acids from samples, for example by sequencing or imaging of labelled molecules as will be explained further below.

### Techniques for Molecular Counting

Any technique for molecular counting known in the art may be used in the context of the present invention, including in particular whole-genome sequencing, exome sequencing, targeted sequencing (including e.g., targeted capture of panels and/or targeted enrichment followed by sequencing), digital molecular counting assays (e.g., digital PCR, sequencing with unique molecular identifiers, direct quantification of targeted fragments labelled by rolling circle replication, etc.), microarrays (e.g., genomic microarrays, custom microarrays, etc.), nanopore sequencing, etc. In particularly convenient embodiments, target sequences are detected by counting products formed by rolling circle replication, e.g., in a rolling circle amplification (RCA) reaction.

Embodiments of the technology implement one or more steps of nucleic acid extraction, MIP probe design, MIP amplification/replication, and/or methods for measuring signal from circularized MIPs. In some embodiments, the MIPs may be immobilized on a surface and detected. Immobilized MIPs may be detected using rolling circle amplification.

In various embodiments, the methods of the technology comprise a target-recognition event, typically comprising hybridization of a target nucleic acid, to another nucleic acid molecule, e.g., a synthetic probe. In specific embodiments, the target recognition event creates conditions in which a representative product is produced (e.g., a probe oligonucleotide that has been extended, ligated, and/or cleaved), the product then being indicative that the target is present in the reaction and that the probe hybridized to it.

A number of different “front-end” methods for recognizing target nucleic acid and producing a new product are described below. For example, a number of ways to produce circularized molecules may be used, for use in a “back end” detection/readout step. These distinctive molecules may be configured to have one or more features useful for capture and/or identification in a downstream backend detection step. Examples of molecules and features produced in a front-end reaction include circularized MIPs having joined sequences (e.g., a complete target-specific sequence formed by ligation of the 3′ and 5′ ends of the probe), having added sequences (e.g., copied portions of a target template) and/or tagged nucleotides (e.g., nucleotides attached to biotin, dyes, quenchers, haptens, and/or other moieties). In some embodiments, the MIPs comprise a feature in a portion of the probe, e.g., in the backbone of the probe. Although the technology is discussed by reference to particular embodiments, such as combinations of certain front-end target-dependent reactions with particular back-end signal amplification methods and detection platforms, e.g., biotin-incorporated MIP coupled with an enzyme-free hybridization chain reaction back-end, the invention is not limited to the particular combinations of front-end and back-end methods and configurations disclosed herein, or to any particular methods of detecting a signal from selected target sequences. It will be appreciated that the skilled person may readily adapt one front-end to work with an alternative back-end. For example, a circularized MIP of may be captured and detected using an enzyme-linked probe, or might alternatively be amplified in a rolling circle amplification assay. In some embodiments, assays are performed in a multiplexed manner. In some embodiments, multiplexed assays can be performed under conditions that allow different loci to reach more similar levels of amplification.

In embodiments of the technology, target sequences are detected using a method for counting circularized nucleic acid probes, comprising: a) providing a ligation mixture comprising circularized nucleic acid probes and linear nucleic acids; b) treating the ligation mixture with at least one exonuclease, wherein circularized nucleic acid probes are not substrate for the at least one exonuclease; c) forming a plurality of complexes, each complex comprising an oligonucleotide primer hybridized to a circularized nucleic acid probe from the treated ligation mixture; d) detecting formation of the plurality of complexes in a process comprising: i) extending primers in the complexes in a rolling circle amplification (RCA) reaction to form RCA products that comprise primer portions; ii) hybridizing labeled probes to the RCA products, wherein RCA products with hybridized labeled probes are localized to a support at dispersed loci, wherein at least a portion of the RCA products localized at the dispersed loci are individually detectable by detection of hybridized labeled probes; and iii) counting RCA products at dispersed loci on the support, in some embodiments the counting RCA products at dispersed loci on the support by microscopy. See, e.g., WO 2019/195346 and WO 2020/206170, each of which is incorporated herein by reference in its entirety.

In some embodiments, the primers are localized at the dispersed loci prior to the extending, while in some embodiments, the primer portions of the RCA products are localized to the dispersed loci after the extending. In any of the embodiments described above, the primers or primer portions may be bound to one or more surfaces, in some embodiments the primer portions are covalently linked to the one or more surfaces. Alternatively, the primers or primer portions may be hybridized to capture oligonucleotides, wherein the capture oligonucleotides are bound to one or more surfaces, in some embodiments the oligonucleotides are covalently linked to the one or more surfaces. In particular embodiments, the primers are bound to the one or more surfaces, in various embodiments they are covalently linked to the one or more surfaces, or are hybridized to capture oligonucleotides bound to the one or more surfaces, in some embodiments they are covalently linked to the one or more surfaces, before the extending.

The support may comprise one or more surfaces selected from a portion of an assay plate, in some embodiments it is a multi-well assay plate, in other embodiments it is a glass-bottom assay plate; a portion of a slide; and one or more particles. In some embodiments, the particles are nanoparticles, in other embodiments the particles are paramagnetic particles, in various embodiments the particles are ferromagnetic nanoparticles, in still other embodiments the particles are iron oxide nanoparticles. The primers may be bound to surfaces on particles, in some embodiments they are covalently linked to surfaces on the particles, and the RCA products with hybridized labeled probes may be localized to dispersed loci by one or more of a magnet, centrifugation, and filtration. In any one of the embodiments described above, the dispersed loci may be in an irregular dispersal or the dispersed loci may be in an addressable array.

Any of the embodiments described above comprise embodiments wherein hybridized labeled probes comprise oligonucleotides comprising a fluorescent label or a quencher moiety, or both a fluorescent label and a quencher moiety. The technology includes but is not limited to embodiments wherein a plurality of RCA products are hybridized to labeled probes that all comprise the same label, in some embodiments they are the same fluorescent label. In various embodiments, a plurality of RCA products are hybridized to labeled probes, that comprise two, three, four, five, six, seven or more different labels, in specific embodiments two, three, four, five, six, seven, or more different fluorescent labels.

In any of the embodiments above, forming RCA products may comprise extending the primers in the complexes in a reaction mixture comprising polyethylene glycol (PEG), in some embodiments the PEG is present in an amount of at least 2 to 10% (w:v), in other embodiments the PEG is present in an amount of at least 12%, in some embodiments the PEG is present in an amount of at least 14%, in still other embodiments the PEG is present in an amount of at least 16%, in some embodiments the PEG is present in an amount of at least 18% to 20% or more PEG. In any of these embodiments, PEG may have an average molecular weight between 200 and 8000, in some embodiments the average molecular weight is between 200 and 1000, in other embodiments the average molecular weight is between 400 and 800, preferably 600.

In any of the embodiments above, forming RCA products may comprise incubating a reaction mixture for an incubation period having a beginning and an end, wherein the reaction mixture is treated by mixing one or more times between the beginning of the incubation period and the end of the incubation period, wherein the mixing comprises one or more of vortexing, bumping, rocking, tilting, and ultrasonic mixing.

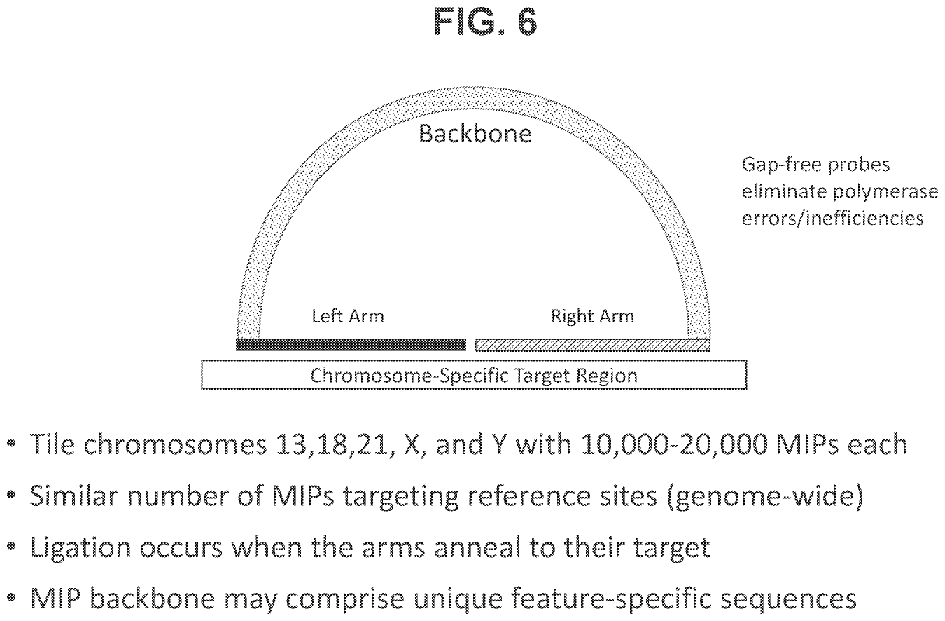

In any of the embodiments above, providing the ligation mixture comprising circularized nucleic acid probes and linear nucleic acids may comprise ligating MIP probes, in various embodiments the probes are padlock probes, in the presence of a target nucleic acid target nucleic acid from a sample, to form the circularized nucleic acid probes. FIG. 6 provides a schematic diagram of a molecular inversion probe (MIP). The molecular inversion probe contains first and second targeting polynucleotide arms that are complementary to adjacent or proximal regions on a target nucleic acid to be detected, with a polynucleotide linker or “backbone” connecting the two arms (see FIG. 6). In the presence of a complementary target nucleic acid, the MIP can be circularized to form a MIP replicon suitable for detection. In some embodiments, the MIP is simply ligated using a nick repair enzyme, e.g., T4 DNA ligase, AMPLIGASE thermostable DNA ligase, etc., while in some embodiments closing of the probe to form a circle comprises additional modification of the probe to create a ligatable nick, e.g., cleavage of an overlap between the termini, filling of a gap between the termini using a nucleic acid polymerase, etc.

A target site or sequence, as used herein, refers to a portion or region of a nucleic acid sequence that is sought to be sorted out from other nucleic acids in the sample that have other sequences, which is informative for determining the presence or absence of a genetic disorder or condition (e.g., the presence or absence of mutations, polymorphisms, deletions, insertions, aneuploidy etc.) and/or for determining the fetal fraction in the sample. In some embodiments, the targeting MIPs comprise in sequence the following components: first targeting polynucleotide arm—first unique targeting molecular tag—polynucleotide linker—second unique targeting molecular tag—second targeting polynucleotide arm. In some embodiments, a target population of the targeting MIPs are used in the methods of the disclosure. In the target population, the pairs of the first and second targeting polynucleotide arms in each of the targeting MIPs are identical and are substantially complementary to first and second regions in the nucleic acid that, respectively, flank the target site. See, e.g., WO 2017/020023 and WO 2017/020024, each of which is incorporated herein by reference in its entirety.

In some embodiments, the length of each of the targeting polynucleotide arms is between 18 and 35 base pairs. In some embodiments, the length of each of the targeting polynucleotide arms is 18, 19, 20, 21, 22, 23, 24, 25, 26, 27, 28, 29, 30, 31, 32, 33, 34, or 35 base pairs, or any size range between 18 and 35 base pairs. In some embodiments, each of the targeting polynucleotide arms has a melting temperature between 55° C. and 70° C. In some embodiments, each of the targeting polynucleotide arms has a melting temperature at 56° C., 57° C., 58° C., 59° C., 60° C., 61° C., 62° C., 63° C., 64° C., 65° C., 66° C., 67° C., 68° C., 69° C., 70° C., or any temperature between 55° C. and 70° C. In some embodiments, each of the targeting polynucleotide arms has a GC content between 20% and 80%. In some embodiments, each of the targeting polynucleotide arms has a GC content of 20-30%, 30-40%, or 30-50%, or 30-60%, or 40-50%, or 40-60%, or 40-70%, or 50-60%, or 50-70%, or 50-80%, or any range of GC content between 20% and 80%, or any specific percentage between 20% and 80%.

In some embodiments, the polynucleotide linker is not substantially complementary to any genomic region of the sample or the subject. In some embodiments, the polynucleotide linker has a length of 30 to 40 base pairs. In some embodiments, the polynucleotide linker has a length of 31, 32, 33, 34, 35, 36, 37, 38, or 39 base pairs, or any interval between 30 and 40 base pairs, and including 30 or 40 base pairs. In some embodiments, the polynucleotide linker has a melting temperature of between 60° C. and 80° C. In some embodiments, the polynucleotide linker has a melting temperature of 60° C., 65° C., 70° C., 75° C., or 80° C., or any interval between 60° C. and 80° C., or any specific temperature between 60° C. and 80° C. In some embodiments, the polynucleotide linker has a GC content between 40% and 60%. In some embodiments, the polynucleotide linker has a GC content of 40%, 45%, 50%, 55%, or 60%, or any interval between 40% and 60%, or any specific percentage between 40% and 60%.

In some embodiments, targeting MIPs replicons are produced by: i) the first and second targeting polynucleotide arms, respectively, hybridizing to the first and second regions in the nucleic acid that, together, form a continuous target site; and ii) after the hybridization, using a ligation reaction mixture to ligate the nick region between the two targeting polynucleotide arms to form single-stranded circular nucleic acid molecules. In other embodiments, targeting MIPs replicons are produced by: i) the first and second targeting polynucleotide arms, respectively, hybridizing to the first and second regions in the nucleic acid that, respectively, flank the target site; and ii) after the hybridization, using a ligation/extension mixture to extend and ligate the gap region between the two targeting polynucleotide arms to form single-stranded circular nucleic acid molecules.

In any of the embodiments above, the at least one exonuclease may comprise one or more of Exonuclease I (Exo I, E. coli), Thermolabile Exonuclease I; Exonuclease VII (Exo VII, E. coli), Exonuclease T (or “RNase T”) and RecJ_{f}, a recombinant fusion protein of E. coli RecJ and maltose binding protein (MBP). In any of these embodiments, treating the ligation mixture with at least one exonuclease may comprise inactivating the at least one exonuclease, in some embodiments this is done by heat-inactivating the at least one exonuclease, prior to forming the plurality of complexes.

In embodiments described above, forming RCA products may comprise extending the primers in the complexes in a reaction mixture that comprises the labeled probes, and/or may comprise embodiments wherein RCA products are localized at the dispersed loci prior to hybridizing the labeled probes to the RCA products. In some embodiments, RCA products with hybridized labeled probes are treated with graphene oxide prior to counting the RCA products at the dispersed loci.

Any of the embodiments above may comprise embodiments wherein RCA products with hybridized labeled probes are treated with one or more detergents prior to counting the RCA products at the dispersed loci. Any of the embodiments above may comprise embodiments wherein the support comprises an organic coating, the coating comprising a polymeric coating polymerized from surface-modifying monomers, wherein the surface-modifying monomers comprise one or more of dopamine, tannic acid, caffeic acid, pyrogallol, gallic acid, epigallocatechin gallate, and epicatechin gallate monomers, y dopamine and tannic acid. In some embodiments, the polymeric coating is homopolymeric. See, e.g., US 2003/0087338, which is incorporated herein by reference for all purposes.

Any of the embodiments above may comprise embodiments wherein prior to localizing RCA products at the dispersed loci, the primers, primer portions, or capture oligonucleotides comprise one or more immobilization moieties. In various embodiments the one or more immobilization moieties are selected from a reactive amine, a reactive thiol group, biotin, and a hapten, wherein the immobilization moieties are exposed to a surface under conditions wherein the immobilization moieties interact with the surface to bind the primers, primer portions, or capture oligonucleotides to the surface. In certain embodiments, prior to localizing RCA products at the dispersed loci the surface comprises at least one of: acrylic groups; thiol-containing groups; reactive amine groups; carboxyl groups, streptavidin, antibodies, haptens, carbohydrates, lectins.

Embodiments of the technology use a method for counting circularized nucleic acid probes, comprising: a) providing a ligation mixture comprising circularized nucleic acid probes and linear nucleic acids; b) forming a plurality of complexes, each complex comprising an oligonucleotide primer hybridized to a circularized nucleic acid probe from the ligation mixture, wherein the primer is bound to a nanoparticle, in some embodiments it is a paramagnetic nanoparticle; c) detecting formation of the plurality of complexes in a process comprising: i) extending primers in the complexes in a rolling circle amplification (RCA) reaction to form RCA products bound to nanoparticles, wherein at least a portion of the RCA products on nanoparticles are individually detectable; and iii) counting RCA products on the nanoparticles.

Some embodiments comprise hybridizing labeled probes to the RCA products, wherein at least a portion of the RCA products are individually detectable by detection of hybridized labeled probes. Any of the embodiments described above comprise embodiments wherein hybridized labeled probes comprise oligonucleotides comprising a fluorescent label or a quencher moiety, or both a fluorescent label and a quencher moiety.

In any of the embodiments wherein the primer is bound to a nanoparticle, the method comprises embodiments wherein the nanoparticles are paramagnetic nanoparticles, in specific embodiments iron oxide nanoparticles. In embodiments the nanoparticles have an average diameter of less than about 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 5 nm, or 1 nm in diameter, wherein the nanoparticles are from 1 to 50 nm, from 5 to 20 nm average diameter. In some embodiments, the nanoparticles comprise an inorganic core of about 2.5 to about 55 nm diameter, and an organic coating, the organic coating having an overall thickness of about 3 to 5 nm. In specific embodiments, the nanoparticles are predominantly spheroid or spherical, and in certain embodiments, the nanoparticles are essentially uniform in diameter.

In any of the embodiments wherein the primer is bound to a nanoparticle include embodiments wherein prior to binding primers, the nanoparticles have a surface comprising reactive groups, the reactive groups. In various embodiments, the reactive group comprises at least one of: acrylic groups; thiol-containing groups; reactive amine groups; carboxyl groups, wherein the primers comprise reactive groups suitable for forming covalent bonds with reactive groups on the surface of the nanoparticles, and wherein the primers and the nanoparticles are treated together under conditions wherein the primers are covalently linked to the nanoparticles.

In any of the embodiments wherein the primer is bound to a nanoparticle, counting RCA products on nanoparticles may comprise at least one of fluorescence microscopy, flow cytometry, and nanopore sensing. In any of the embodiments wherein the primer is bound to a nanoparticle, counting RCA products on nanoparticles may comprise localizing RCA products to a support at dispersed loci wherein at least a portion of the RCA products localized at the dispersed loci are individually detectable by detection of hybridized labeled probes and counting RCA products at dispersed loci on the support. In some embodiments, RCA products with hybridized labeled probes are localized to dispersed loci by one or more of a magnet, centrifugation, and filtration. Any of the embodiments wherein the primer is bound to a nanoparticle include embodiments wherein prior to forming the plurality of complexes, the ligation mixture is treated with at least one exonuclease, wherein circularized nucleic acid probes are not substrate for the at least one exonuclease. In specific embodiments, the at least one exonuclease comprises at least one exonuclease selected from Rec Jf, Exo VII, Exo T, and Thermolabile Exo I.

Embodiments of the technology use a composition comprising a plurality of complexes bound to a surface of an organic coating on one or more supports, wherein the one or more supports compromise one or more of an assay plate, a glass-bottom assay plate, and a nanoparticle, a paramagnetic nanoparticle, a ferromagnetic nanoparticle, an iron oxide nanoparticle, each complex comprising an oligonucleotide primer hybridized to a circularized nucleic acid probe, wherein the primer is bound to the surface of the organic coating on the support, and a reaction mixture comprising: Phi29 DNA polymerase, at least 0.2 units per μL, at least 0.8 units per μL of Phi29 DNA polymerase; a buffer; a mixture of dNTPs, at least 400 μM, y at least 600 μM, at least 800 μM total dNTPs; PEG, at least 2 to 10% (w:v), at least 12%, at least 14%, at least 16%, or at least 18% to 20% or more PEG. The PEG may have an average molecular weight between 200 and 8000, between 200 and 1000, between 400 and 800, or 600.

Embodiments include any of the compositions described above, wherein the reaction mixture further comprises at least one labeled probe, a fluorescently labeled probe, a molecular beacon probe, least 100 nM of labeled probe, or at least 1000 nM of labeled probe.

Embodiments of the technology further use a composition comprising a plurality of RCA products bound to a surface of an organic coating on one or more supports, wherein the one or more supports comprise one or more of an assay plate, a glass-bottom assay plate, and a nanoparticle, a paramagnetic nanoparticle, a ferromagnetic nanoparticle, or an iron oxide nanoparticle, each RCA product comprising a primer portion bound to the surface of the organic coating on the support, and a buffer comprising Mg⁺⁺, the solution further comprising: one or more labeled probes hybridized to RCA products; and one or more of: graphene oxide; one or more detergents.

Embodiments of such compositions include embodiments wherein the labeled probes comprise fluorescent labels and embodiments wherein the labeled probes comprise quencher moieties.

Any of the embodiments above include embodiments of the composition wherein the solution comprising a labeled probe comprises a fluorescently labeled probe, a molecular beacon probe, more than 100 nM of labeled probe, at least 1000 nM of labeled probe, and/or wherein the buffer comprising Mg⁺⁺ is a Phi29 DNA polymerase buffer.

Embodiments of the technology comprise systems, for example, a system comprising: i) a plurality of complexes bound to a surface of an organic coating on one or more supports, wherein the one or more supports comprise one or more of an assay plate, a glass-bottom assay plate, and a nanoparticle, a paramagnetic nanoparticle, a ferromagnetic nanoparticle, or an iron oxide nanoparticle, each complex comprising an oligonucleotide primer hybridized to a circularized nucleic acid probe, wherein the primer is bound to the surface of the organic coating on the support; ii) DNA polymerase, Phi29 DNA polymerase; iii) one or more labeled probes, or y fluorescently labeled probes. In some embodiments, a system further comprises one or more of: iv) a buffer comprising Mg++, a buffer comprising MgCl₂, a Phi29 DNA polymerase buffer; v) PEG, PEG having an average molecular weight between 200 and 8000, between 200 and 1000, between 400 and 800, or 600; vi) one or more detergents, vii) as solution comprising dNTPs; and viii) graphene oxide. In some embodiments, the organic coating is a polymeric coating polymerized from surface-modifying monomers, wherein the surface-modifying monomers comprise one or more of dopamine, tannic acid, caffeic acid, pyrogallol, gallic acid, epigallocatechin gallate, and epicatechin gallate monomers, dopamine and tannic acid, and in some embodiments, the polymeric coating is homopolymeric.

In some embodiments, the technology use support with a first surface that has been modified with one or more surface modifying agent(s) (SMA(s)), thereby providing a support comprising a second surface (or coating). In various embodiments, the second surface (or coating) comprises functional groups capable of forming complexes with one or more analytes. Thus, in some embodiments, the support is referred to herein as a “surface functionalized substrate” (SFS). In some embodiments, the functional groups capable of complexing with the one or more analytes is an amine group (e.g., a primary, secondary, tertiary or quaternary amine), a carboxylate or carboxylic acid group, or a combination thereof. In some embodiments, at least one of the one or more SMAs is a vinyl monomer. In embodiments, the vinyl monomer can comprise an acrylate monomer. In embodiments, the acrylate monomer comprises acrylic acid, methacrylate, ethyl acrylate, propyl acrylate, a butyl acrylate, or a combination thereof. In some embodiments, the acrylate monomer comprises 2-aminoethyl methacrylate (AEMA), acrylic acid (AA), or a combination thereof. In some embodiments, at least one of the one or more SMAs is a phenol monomer (i.e., a monomer comprising a phenol group). In some embodiments, modifying the first surface comprises polymerizing the one or more SMAs in the presence of the first surface. Thus, in some embodiments, modifying the first surface comprises contacting the first surface with a mixture comprising a carrier and one or more SMAs, wherein the one or more SMAs polymerizes in the presence of the first surface, thereby providing the second surface. In some more particular embodiments, the mixture further comprises one or more initiators, wherein the initiator(s) initiate polymerization of the one or more SMAs. In some embodiments, the initiator is ammonium persulfate, TEMED, or a combination thereof. In some embodiments, the mixture comprises one SMA and the polymerization provides a homopolymer. In other embodiments, the mixture comprises at least two SMAs and the polymerization provides a copolymer. The homopolymer or the copolymer forms or is deposited on the first surface, thereby providing the second surface. In some embodiments, the polymerization or copolymerization of the SMA(s) can be performed in the presence of an initiator. In some embodiments, SMAs comprise photopolymers and polymerization is initiated by light, e.g., from a halogen, argon, xenon or LED light source. In some more particular embodiments, preparing the support comprises a) providing a substrate having a first surface; b) modifying the first surface by contacting the first surface with a mixture comprising a carrier, a first SMA which is dopamine, a second SMA which is AEMA, and one or more initiators; c) thereby providing a support comprising a second surface, wherein the second surface comprises a copolymer derived from the dopamine and the AEMA, and wherein the support is a surface functionalized substrate. In some embodiments, the mixture is an aqueous solution. In some embodiments, the first surface is a silanized surface, such as glass. In some other embodiments, the first surface comprises an organic polymer, such as polystyrene. In some more particular embodiments, preparing the support comprises a) providing a substrate having a first surface; b) modifying the first surface by contacting the first surface with a mixture comprising a carrier, a first SMA which is dopamine, a second SMA which is acrylic acid, and one or more initiators; c) thereby providing a support comprising a second surface, wherein the second surface comprises a copolymer derived from the dopamine and the acrylic acid, and wherein the support is a surface functionalized substrate. In some embodiments, the mixture is an aqueous solution. In some embodiments, the first surface is a silanized surface, such as glass. In some other embodiments, the first surface comprises an organic polymer, such as polystyrene. In some more particular embodiments, the method of preparing the support comprises a) providing a substrate having a first surface; b) modifying the first surface by contacting the first surface with a mixture comprising a carrier, a first SMA which is tannic acid, a second SMA which is AEMA, and one or more initiators; c) thereby providing a support comprising a second surface, wherein the second surface comprises a copolymer derived from the tannic acid and the AEMA, and wherein the support is a surface functionalized substrate. In some more particular embodiments, preparing the support comprises a) providing a substrate having a first surface; b) modifying the first surface by contacting the first surface with a mixture comprising a carrier, a first SMA which is tannic acid, a second SMA which is acrylic acid, and one or more initiators; c) thereby providing a support comprising a second surface, wherein the second surface comprises a copolymer derived from the tannic acid and the acrylic acid, and wherein the support is a surface functionalized substrate

In some embodiments, the technology uses a method for counting target molecules on a support, comprising: a) providing a first surface; b) modifying the first surface with at least one SMA to provide a surface functionalized substrate (SFS); optionally, the SFS comprises functional groups selected from at least one of carboxylate, carboxylic acid and amine groups; c) contacting the SFS with one or more analytes; d) thereby forming a plurality of complexes between the functional groups on the SFS and the one or more analytes; and e) counting the plurality of complexes. In some embodiments, the first surface (or substrate) is a silanized surface. In some embodiments, the silanized surface is glass, while in some embodiments, the surface is unsilanized glass. In certain embodiments, the silanized surface comprises a surface treated with 3-aminopropyltriethoxysilane or 3-(trimethoxysilyl) propyl methacrylate. See, e.g., WO 2019/195346 A1 to Sekedat, et al., Methods, Systems, and Compositions for Counting Nucleic Acids (2019), which is incorporated herein by reference in its entirety, for all purposes. In some embodiments, the one or more analytes comprises at least one of an RCA product comprising a plurality of hybridized labeled probes and a double-stranded scaffold product comprising a plurality of concatemerized labeled scaffold oligonucleotides, wherein formation of a complex is indicative of the presence of a target molecule on the glass surface, and wherein forming said plurality of complexes comprises exposing the glass surface to a solution comprising graphene oxide. The surfaces are not limited to any particular format. For example, in any of the embodiments of described above, the support may comprise a surface in an assay plate, or a glass-bottom assay plate. In some embodiments, the assay plate is a multi-well assay plate, or a microtiter plate.

In some embodiments of the technology, the primer of any of the embodiments described above is bound directly to the support, in some embodiments it is covalently linked to the support. For example, in some embodiments, the primer comprises a biotin moiety and the support comprises avidin, or streptavidin. In some embodiments, the primer is covalently linked to a support by conjugation of an amide bond between an amine and carboxylic acid.

In any of the embodiments described herein, forming a complex or plurality of complexes may comprise exposing the support to a solution comprising a crowding agent. In some embodiments, the crowding agent comprises polyethylene glycol (PEG), at least 2 to 10% (w:v), pat least 12%, at least 14%, at least 16%, or y at least 18% to 20% or more PEG (e.g., 22% PEG). In certain preferred embodiments, the PEG has an average molecular weight between 200 and 8000, between 200 and 1000, between 400 and 800, or 600. In any of the embodiments described above, forming a complex or plurality of complexes may comprise a step of exposing the support to a solution comprising graphene oxide. In preferred embodiments, the support is exposed to graphene oxide prior to step detecting hybridized labeled probe. In particularly preferred embodiments, the support is exposed to a solution that comprises a mixture of labeled probe and graphene oxide. In some embodiments, the support or the glass surface exposed to a solution comprising graphene oxide is washed with a solution comprising one or more detergents prior to the detecting or counting. In certain embodiments, the one or more detergents comprises Tween 20.

In any of the embodiments described above, forming a complex or plurality of complexes may comprise comprising a step of exposing the support to a solution comprising one or more detergents or surfactants. In some embodiments, the support is exposed to a solution comprising one or more detergents or surfactants prior to a step of detecting hybridized labeled probe. In certain embodiments, the support is exposed to a solution that comprises a mixture of labeled probe and one or more detergents or surfactants. In some embodiments, the support or the glass surface is washed with a solution comprising one or more detergents or surfactants. In some embodiments, the detergent comprises an agent selected from anionic agents (e.g., sodium dodecyl sulfate; sodium lauryl sulfate; ammonium lauryl sulfate), cationic agents (e.g., benzalkonium chloride; cetyltrimethylammonium bromide; linear alkylbenzene sulfonates, such as sodium dodecylbenzene sulfonate), non-ionic agents (e.g., a TWEEN detergent, such as polyoxyethylene (20) sorbitan-monolaurate; -monopalmitate; -monostearate; or -monooleate; a TRITON, such as polyethylene glycol p-(1,1,3,3-tetramethylbutyl)-phenyl ether, or TRITON X-100; steroid and steroidal glycosides such as saponin and digitonin), and zwitterionic agents (e.g., CHAPS, which is 3-[(3-cholamidopropyl)dimethylammonio]-1-propanesulfonate), mixtures of detergent agents (e.g., TEEPOL® 610 S detergent, comprising sodium dodecylbenzene sulfonate, sodium C12-C15 alcohol ether sulfate), or a mixture thereof.

Any of the embodiments described herein may comprise forming an RCA product in a process comprises extending a primer on a circularized nucleic acid probe in a reaction mixture. In various embodiments, the reaction mixture comprises at least 0.2 units per μL, preferably at least 0.8 units per μL of Phi29 DNA polymerase and at least 400 μM, at least 600 μM, or at least 800 μM total dNTPs. In some embodiments, forming an RCA product comprising a plurality of hybridized labeled probes comprises forming the RCA product in a reaction mixture that further comprises more than 10 nM fluorescently-labeled oligonucleotide, e.g., a molecular beacon probe, at least 100 nM fluorescently-labeled oligonucleotides probe, or at least 1000 nM fluorophore-labeled probe in the reaction mixture.

In some embodiments, forming an RCA product comprising a plurality of hybridized labeled probes comprises forming the RCA product in a reaction mixture that does not comprise labeled probe, then treating the RCA product on the support with a solution that comprises one more labeled probes, or a solution that comprises Mg⁺⁺, or MgCl₂. In some embodiments, RCA product is removed from the reaction mixture, and in some embodiments washed, e.g., with a buffer, prior to treatment with the solution comprising one or more labeled probes.

In any of the embodiments described herein, complexes immobilized on a surface may comprise at least one polypeptide, e.g., an antibody, a lectin, and/or they may comprise at least one specifically-bindable molecule selected from a hapten, a carbohydrate, and a lipid.

In some embodiments of the technology, forming an RCA product comprises incubating the reaction mixture at least 37° C., at least 42° C., or at least 45° C. In certain embodiments, the reaction mixture comprises PEG, at least 2 to 10% (w:v), y at least 12%, at least 14%, at least 16%, or at least 18% to 20% PEG.

In some embodiments, the technology uses a composition comprising a silanized surface or non-silanized surface. In some embodiments, a surface modified using one or more surface modifying agents to provide a second surface bound to a plurality of complexes, each comprising an oligonucleotide primer hybridized to a circularized nucleic acid probe, wherein the primer is localized to a support, and a reaction mixture comprising at least 0.1 units per μL, at least 0.2 to 0.8 units per μL of Phi29 DNA polymerase; a buffer; at least 400 μM, at least 600 μM, or at least 800 μM total dNTP; and PEG, at least 2 to 20% (w:v), 12 to 18%, 14 to 16%, or 15% PEG. In some embodiments, the PEG has an average molecular weight of between 200 and 8000, between 200 and 1000, between 400 and 800, or about 600. In some embodiments, the reaction mixture further comprises at least 10 nM fluorescently labeled oligonucleotide, e.g., molecular beacon probe, at least 100 nM fluorescently labeled oligonucleotide, or at least 1000 nM fluorescently labeled oligonucleotide. In some embodiments, RCA product is removed from the reaction mixture, and in some embodiments washed, e.g., with a buffer, prior to treatment with the solution comprising one or more labeled probes.

In some embodiments of the composition, the primers are localized to the support in an irregular dispersal, while in some embodiments, the primers are localized to the support in an addressable array. In certain embodiments, the primer is covalently linked to the support, while in some embodiments, wherein the primer comprises a biotin moiety and the support comprises avidin, or streptavidin. In other embodiments, the primer is covalently bound to a bead or particle, a small nanoparticle, or a paramagnetic small nanoparticle, and the nanoparticle-bound primer is localized to a surface by an application of force, e.g., with a magnet or centrifuge In some embodiments, the complexes comprise an antibody bound to an antigen or hapten and in some embodiments, the complexes comprise an antigen or hapten bound directly to the support. In some embodiments, the antigen or hapten is covalently attached to the support.

Embodiments of the composition described above may comprise a silanized surface bound to a plurality of complexes each comprising an RCA product comprising a plurality of hybridized labeled probes, and a solution comprising graphene oxide. In some embodiments, the silanized surface is glass. In some specific embodiments, the silanized surface comprises a surface, or a glass surface, treated with 3-aminopropyltriethoxysilane or 3-(trimethoxysilyl) propyl methacrylate. In some embodiments, the surface, or glass surface is not silanized. In certain embodiments, the surface comprises a polymeric coating formed by polymerization of one or more monomers, including but not limited to e.g., tannic acid, acrylic acid, dopamine, etc. In some embodiments, the support comprises a surface comprising polytannic acid or polydopamine. In some embodiments, the solution comprising graphene oxide further comprises a fluorescently labeled probe, e.g., a molecular beacon probe, more than 10 nM of fluorescently labeled probe, at least 100 nM fluorescently labeled probe, or at least 1000 nM fluorescently labeled probe. In some embodiments of the composition, the solution comprising graphene oxide comprises a buffer solution comprising MgCl₂. In certain embodiments, the buffer comprising MgCl₂ is a Phi29 DNA polymerase buffer.

Circular DNA molecules such as ligated MIPs are suitable substrates for amplification using rolling circle amplification (RCA). In certain embodiments of RCA, a rolling circle replication primer hybridizes to a circular nucleic acid molecule, e.g., a ligated MIP, or circularized cfDNA. Extension of the primer using a strand-displacing DNA polymerase (e.g., φ29 (Phi29), Bst Large Fragment, and Klenow fragment of E. coli Pol I DNA polymerases) results in long single-stranded DNA molecules containing repeats of a nucleic acid sequence complementary to the MIP circular molecule.

In some embodiments, ligation-mediated rolling circle amplification (LM-RCA), which involves a ligation operation prior to replication, is utilized. In the ligation operation, a probe hybridizes to its complementary target nucleic acid sequence, if present, and the ends of the hybridized probe are joined by ligation to form a covalently closed, single-stranded nucleic acid. After ligation, a rolling circle replication primer hybridizes to probe molecules to initiate rolling circle replication, as described above. Generally, LM-RCA comprises mixing an open circle probe with a target sample, resulting in an probe-target sample mixture, and incubating the probe-target sample mixture under conditions promoting hybridization between the open circle probe and a target sequence, mixing ligase with the probe-target sample mixture, resulting in a ligation mixture, and incubating the ligation mixture under conditions promoting ligation of the open circle probe to form an amplification target circle (ATC, which is also referred to an RCA replicon). A rolling circle replication primer (RCRP) is mixed with the ligation mixture, resulting in a primer-ATC mixture, which is incubated under conditions that promote hybridization between the amplification target circle and the rolling circle replication primer. DNA polymerase is mixed with the primer-ATC mixture, resulting in a polymerase-ATC mixture, which is incubated under conditions promoting replication of the amplification target circle, where replication of the amplification target circle results in formation of tandem sequence DNA (TS-DNA), i.e., a long strand of single-stranded DNA that contains a concatemer of the sequence complementary to the amplification target circle.

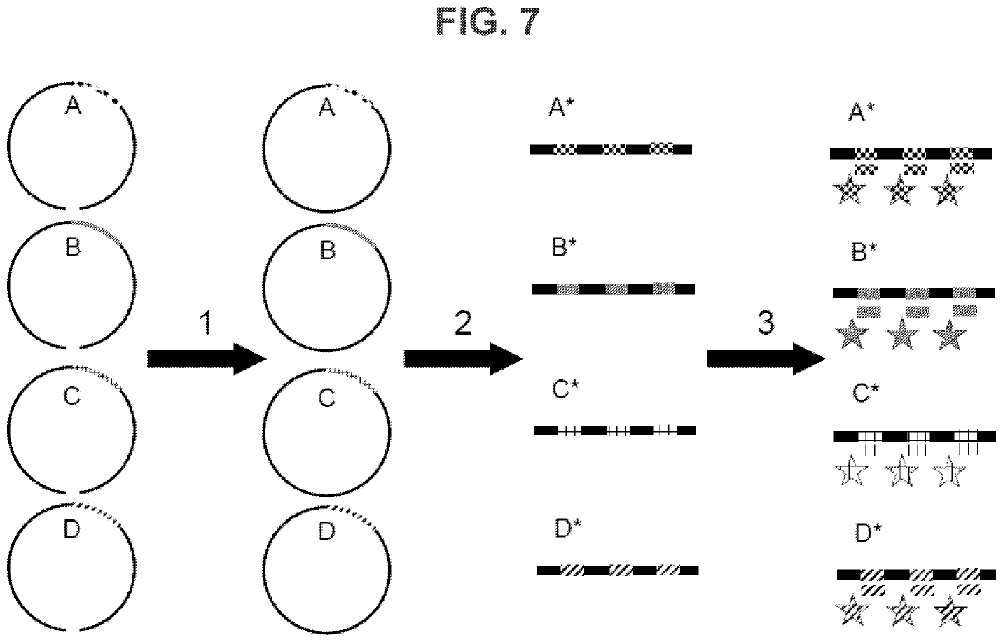

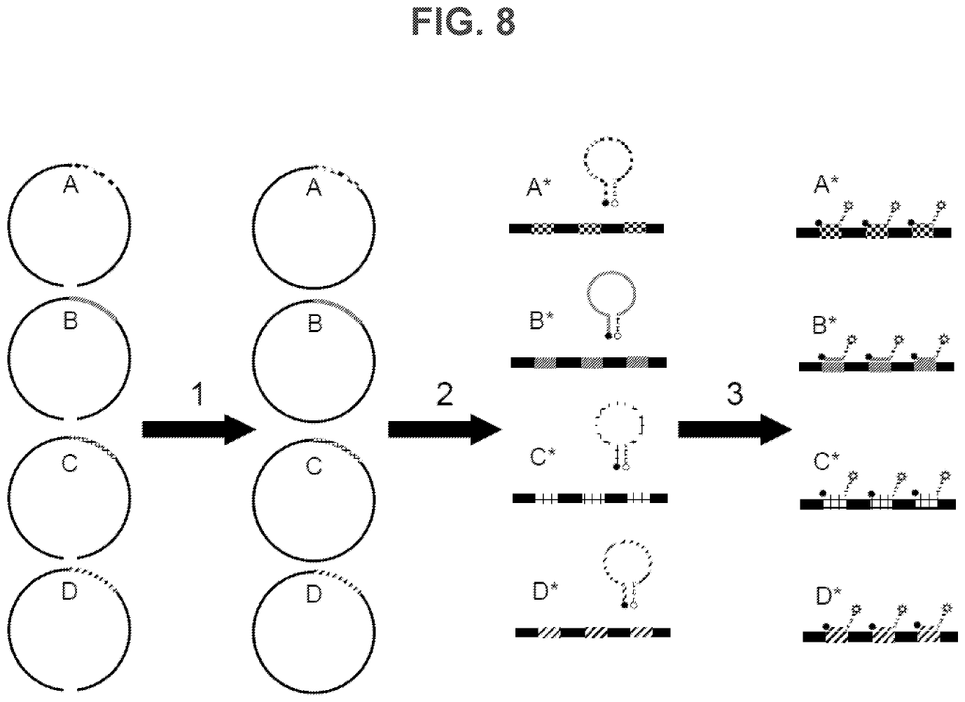

In the embodiment illustrated in FIG. 7, circularized molecules A, B, C, and D consist of MIPs that are specific to different genomic regions, such as e.g., regions on chromosome 1, 13, 18, 21, X, and/or Y. The sequence of the MIP surrounding the gap complements region of the targeted chromosome, and the backbone of the MIP contains a specific sequence that is used to hybridize a probe that will contain a specific fluorescent dye (FITC, ALEXA, Dylight, Cyan, Rhodamine dyes, quantum dots, etc.). Step 1 comprises hybridizing the MIPs to cfDNA, a single base pair extension (or longer extension), and ligation to circularize the extended MIP. Step 2 comprises rolling circle amplification of the circularized MIP so that the sequence required to hybridize to the fluorescently labeled oligonucleotide is amplified. A*, B*, C*, D* are the complement of the MIP sequence. Step 3 comprises hybridizing the fluorescently labeled probe to the rolling circle product. In the embodiment illustrated in FIG. 8, detection of the RCA product is facilitated by molecular probes instead of fluorescent dye labeled oligonucleotides.

There are multiple ways to immobilize the MIP to a surface (e.g., a bead or glass surface) For example, this may be accomplished by priming the rolling circle amplification with a modified oligonucleotide comprising a bindable moiety. Groups useful for modification of the priming oligonucleotide include but are not limited to thiol, amino, azide, alkyne, and biotin, such that the modified oligonucleotides can be immobilized using appropriate reactions, e.g., as outlined in Meyer et. al., “Advances in DNA-mediated immobilization” Current Opinions in Chemical Biology, 18:8: 8-15 (2014), which is incorporated herein by reference in its entirety, for all purposes.

Imaging of the fluorescent dye incorporated MIPs can be accomplished by using methods comprising immobilization of MIPs to a surface (e.g., glass slide or bead), e.g., using modifications of the MIP backbone to contain modified bases that can be immobilized using appropriate reactions as outlined above and in Meyer et. al., supra. and detected using an antibody. Once immobilized to a surface, an antibody directed to an incorporated tag can be used to form antibody-MIP complexes that can be imaged with microscopy. In some embodiments, the antibody may be conjugated to enhance or amplify detectable signal from the complexes. For example, conjugation of β-galactosidase to the antibody allows detection in a single molecule array (“SIMOA”), using the process described by Quanterix, wherein each complex is immobilized on a bead such that any bead has no more than one labeled immunocomplex, and the beads are distributed to an array of femtoliter-sized wells, such that each well contains, at most, one bead. With addition of resorufin-β-galactopyranoside, the β-galactosidase on the immobilized immunocomplexes catalyzes the production of resorufin, which fluoresces. Upon visualization, the fluorescence emitted in wells having an immobilized individual immunocomplexes can be detected and counted. See, e.g., Quanterix Whitepaper 1.0, Scientific Principle of Simoa (Single Molecule Array) Technology, 1-2 (2013); and Quanterix Whitepaper 6.0, Practical Application of Simoa™ HD-1 Analyzer for Ultrasensitive Multiplex Immunodetection of Protein Biomarkers, 1-3 (2015), each of which is incorporated herein by reference for all purposes In some embodiments, the antibody-MIP complex may be directly detected, e.g., using a solid state nanopore with an antibody labeled with poly(ethylene glycol) at various of molecular weights, as described in Morin et. al., “Nanopore-Based Target Sequence Detection” PLOS One, DOI:10.1371/journal.pone.0154426 (2016), incorporated herein by reference.

Many options exist for detection and quantitation of fluorescence signal from the embodiments of the technology described hereinabove. Detection can be based on measuring, for example physicochemical, electromagnetic, electrical, optoelectronic or electrochemical properties, or characteristics of the immobilized molecule and/or target molecule. Two factors that are pertinent to single molecule detection of molecules on a surface are achieving sufficient spatial resolution to resolve individual molecules, and distinguishing the desired single molecules from background signals, e.g., from probes bound non-specifically to a surface. Exemplary methods for detecting single molecule-associated signals are found, e.g., in WO 2016/134191, which is incorporated by reference herein in its entirety for all purposes. In some embodiments, assays are configured for standard SBS micro plate detection, e.g., in a SpectraMax microplate reader or other plate reader. While this method typically requires low-variance fluorescence (multiple wells, multiple measurements), this format can be multiplexed and read on multiple different fluorescence channels. Additionally, the format is very high throughput.

Embodiments can also be configured for detection on a surface, e.g., a glass, gold, or carbon (e.g., diamond) surface. In some embodiments, signal detection is done by any method for detecting electromagnetic radiation (e.g., light) such as a method selected from far-field optical methods, near-field optical methods, epi-fluorescence spectroscopy, confocal microscopy, two-photon microscopy, optical microscopy, and total internal reflection microscopy, where the target molecule is labelled with an electromagnetic radiation emitter. Other methods of microscopy, such as atomic force microscopy (AFM) or other scanning probe microscopies (SPM) are also appropriate. In some embodiments, it may not be necessary to label the target. Alternatively, labels that can be detected by SPM can be used. In some embodiments, signal detection and/or measurement comprises surface reading by counting fluorescent clusters using an imaging system such as an ImageXpress imaging system (Molecular Devices, San Jose, CA), and similar systems.

Embodiments of the technology may be configured for detection using many other systems and instrument platforms, e.g., bead assays (e.g., Luminex), array hybridization, NanoString nCounter single molecule counting device. See, e.g., G K Geiss, et al., Direct multiplexed measurement of gene expression with color-coded probe pairs; Nature Biotechnology 26(3):317-25 (2008), U.S. Patent Publication 2018/0066309 A1 published Mar. 8, 2018, (P N Hengen, et. Al., Invent., Nanostring Technogies, Inc.), etc.

In the Luminex bead assay, color-coded beads, pre-coated with analyte-specific capture antibody for the molecule of interest, are added to the sample. Multiple analytes can be simultaneously detected in the same sample. The analyte-specific antibodies capture the analyte of interest. Biotinylated detection antibodies that are also specific to the analyte of interest are added, such that an antibody-antigen sandwich is formed. Phycoerythrin (PE)-conjugated streptavidin is added, and the beads are read on a dual-laser flow-based detection instrument. The beads are read on a dual-laser flow-based detection instrument, such as the Luminex 200™ or Bio-Rad® Bio-Plex® analyzer. One laser classifies the bead and determines the analyte that is being detected. The second laser determines the magnitude of the PE-derived signal, which is in direct proportion to the amount of bound analyte.

The NanoString nCounter is a single-molecule counting device for the digital quantification of hundreds of different genes in a single multiplexed reaction. The technology uses molecular “barcodes”, each of which is color-coded and attached to a single probe corresponding to a gene (or other nucleic acid) of interest, in combination with solid-phase hybridization and automated imaging and detection. See, e.g. Geiss, et al., supra, which describes use of unique pairs of capture and reporter probes constructed to detect each nucleic acid of interest. In the embodiment described, probes are mixed together with the nucleic acid, e.g., unpartitioned cfDNA, or total RNA from a sample, in a single solution-phase hybridization reaction. Hybridization results in the formation of tripartite structures composed of a target nucleic acid bound to its specific reporter and capture probes, and unhybridized reporter and capture probes are removed e.g., by affinity purification. The hybridization complexes are exposed to an appropriate capture surface, e.g., a streptavidin-coated surface when biotin immobilization tags are used. After capture on the surface, an applied electric field extends and orients each complex in the solution in the same direction.

The complexes are then immobilized in the elongated state and are imaged. Each target molecule of interest can thus be identified by the color code generated by the ordered fluorescent segments present on the reporter probe and tallied to count the target molecules.

In some embodiments, a back-end process configured for single molecule visualization is used. For example, as described above, is the Quanterix platform uses an array of femtoliter-sized wells that capture beads having no more than one tagged complex, with the signal from the captured complexes developed using a resorufin-β-galactopyranoside/0-galactosidase reaction to produce fluorescent resorufin. Visualization of the array permits detection of the signal from each individual complex. In certain embodiments, a solid state nanopore device, e.g., as described by Morin, et al., (see “Nanopore-Based Target Sequence Detection” PLoS ONE 11(5):e0154426 (2016)), is used. A solid-state nanopore is a nano-scale opening formed in a thin solid-state membrane that separates two aqueous volumes. A voltage-clamp amplifier applies a voltage across the membrane while measuring the ionic current through the open pore. When a single charged molecule such as a double-stranded DNA is captured and driven through the pore by electrophoresis, the measured current shifts, and the shift depth (δI) and duration are used to characterize the event. (Morin, et al., supra). Although DNA alone is detectable using this system, distinctive tags (e.g., different sizes of polyethylene glycol (PEG)) may be attached to highly sequence-specific probes (e.g., peptide nucleic acid probes, PNAs) to give any particular DNA-PNA-PEG complex a distinctive signature that represents the target nucleic acid detected in the front-end of the assay.

In a rolling circle amplification reaction, a complex may be formed comprising an oligonucleotide primer and a circular probe, such as a MIP or ligated padlock probe. Extension of the primer in a rolling circle amplification reaction produces long strand of single-stranded DNA that contains a concatemer of the sequence complementary to the circular probe. The RCA product may bind to a plurality of molecular beacon probes having a fluorophore and a quencher. Hybridization of the beacons separates the quencher from the fluorophore, allowing detection of fluorescence from the beacon. Accumulation of the RCA product may be monitored in real time by measuring an increase in fluorescence intensity that is indicative of binding of the beacons to the increasing amount of product over the time course of the reaction.

### Applications

The present methods may find use in any context where it is desirable to determine the fetal fraction in a mixed maternal-fetal cfDNA sample. Thus, the present method may in particular find use in the detection of a prenatal or pregnancy-related disease or condition.

As used herein, the term “prenatal or pregnancy-related disease or condition” refers to any disease, disorder, or condition affecting a pregnant woman, embryo, or fetus. Prenatal or pregnancy-related conditions can also refer to any disease, disorder, or condition that is associated with or arises, either directly or indirectly, as a result of pregnancy. These diseases or conditions can include any and all birth defects, congenital conditions, or hereditary diseases or conditions. Examples of prenatal or pregnancy-related diseases include, but are not limited to, Rhesus disease, hemolytic disease of the newborn, beta-thalassemia, sex determination, determination of pregnancy, a hereditary Mendelian genetic disorder, chromosomal aberrations, a fetal chromosomal aneuploidy, fetal chromosomal trisomy, fetal chromosomal monosomy, trisomy 8, trisomy 13 (Patau Syndrome), trisomy 16, trisomy 18 (Edwards syndrome), trisomy 21 (Down syndrome), X-chromosome linked disorders, trisomy X (XXX syndrome), monosomy X (Turner syndrome), XXY syndrome, XYY syndrome, XYY syndrome, XXXY syndrome, XXYY syndrome, XYYY syndrome, XXXXX syndrome, XXXXY syndrome, XXXYY syndrome, XXYYY syndrome, Fragile X Syndrome, fetal growth restriction, cystic fibrosis, a hemoglobinopathy, fetal death, fetal alcohol syndrome, sickle cell anemia, hemophilia, Klinefelter syndrome, dup(17)(p11.2p1.2) syndrome, endometriosis, Pelizaeus-Merzbacher disease, dup(22)(q11.2q11.2) syndrome, cat eye syndrome, cri-du-chat syndrome, Wolf-Hirschhorn syndrome, Williams-Beuren syndrome, Charcot-Marie-Tooth disease, neuropathy with liability to pressure palsies, Smith-Magenis syndrome, neurofibromatosis, Alagille syndrome, Velocardiofacial syndrome, DiGeorge syndrome, steroid sulfatase deficiency, Prader-Willi syndrome, Kallmann syndrome, microphthalmia with linear skin defects, adrenal hypoplasia, glycerol kinase deficiency, Pelizaeus-Merzbacher disease, testis-determining factor on Y, azospermia (factor a), azospermia (factor b), azospermia (factor c), 1p36 deletion, phenylketonuria, Tay-Sachs disease, adrenal hyperplasia, Fanconi anemia, spinal muscular atrophy, Duchenne's muscular dystrophy, Huntington's disease, myotonic dystrophy, Robertsonian translocation, Angelman syndrome, tuberous sclerosis, ataxia telangieltasia, open spina bifida, neural tube defects, ventral wall defects, small-for-gestational-age, congenital cytomegalovirus, achondroplasia, Marfan's syndrome, congenital hypothyroidism, congenital toxoplasmosis, biotinidase deficiency, galactosemia, maple syrup urine disease, homocystinuria, medium-chain acyl Co-A dehydrogenase deficiency, structural birth defects, heart defects, abnormal limbs, club foot, anencephaly, arhinencephaly/holoprosencephaly, hydrocephaly, anophthalmos/microphthalmos, anotia/microtia, transposition of great vessels, tetralogy of Fallot, hypoplastic left heart syndrome, coarctation of aorta, cleft palate without cleft lip, cleft lip with or without cleft palate, oesophageal atresia/stenosis with or without fistula, small intestine atresia/stenosis, anorectal atresia/stenosis, hypospadias, indeterminate sex, renal agenesis, cystic kidney, preaxial polydactyly, limb reduction defects, diaphragmatic hernia, blindness, cataracts, visual problems, hearing loss, deafness, X-linked adrenoleukodystrophy, Rett syndrome, lysosomal disorders, cerebral palsy, autism, aglossia, albinism, ocular albinism, oculocutaneous albinism, gestational diabetes, Arnold-Chiari malformation, CHARGE syndrome, congenital diaphragmatic hernia, brachydactlia, aniridia, cleft foot and hand, heterochromia, Dwarnian ear, Ehlers Danlos syndrome, epidermolysis bullosa, Gorham's disease, Hashimoto's syndrome, hydrops fetalis, hypotonia, Klippel-Feil syndrome, muscular dystrophy, osteogenesis imperfecta, progeria, Smith Lemli Opitz syndrome, chromatelopsia, X-linked lymphoproliferative disease, omphalocele, gastroschisis, pre-eclampsia, eclampsia, pre-term labor, premature birth, miscarriage, delayed intrauterine growth, ectopic pregnancy, hyperemesis gravidarum, morning sickness, or likelihood for successful induction of labor.

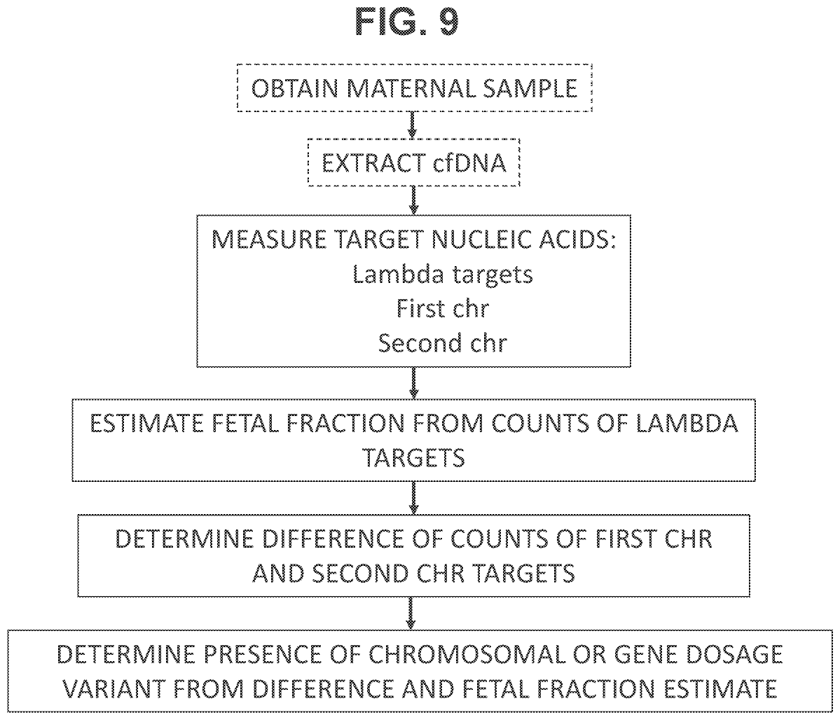

In some embodiments, the technology finds use in analysis of chromosomal aberrations, e.g., aneuploidy, in the context of non-invasive prenatal testing. For example, as illustrated on FIG. 9, some embodiments of applications of the technology comprise obtaining a maternal sample that comprises both maternal and fetal genetic material, and measuring a plurality of target nucleic acids, wherein the target nucleic acids comprise: (i) specific sequences that correlate with fetal fraction, (ii) specific sequences associated with a first chromosome, wherein the first chromosome is suspected of being variant (e.g., in gene dosage or chromosome count) in the fetal material, and (iii) specific sequences associated with a second chromosome, which is not suspected of being variant in the fetal material. The method comprises analyzing an amount of the specific sequences that correlate with fetal fraction, an amount of the target nucleic acids associated with the first chromosome and an amount of target nucleic acids associated with the second chromosome in the sample to determine whether the amount of the target nucleic acids associated with the first chromosome differs sufficiently from the amount the target nucleic acid associated with the second chromosome to indicate a chromosomal or gene dosage variant in the fetus, wherein the assessment of whether the amounts associated with the first and second chromosomes differ sufficiently from each other is based at least in part on an estimate of the fetal DNA fraction of the sample that is based on the amount of the specific sequences that correlate with fetal fraction. In various embodiments, the target nucleic acids associated with the first and second chromosomes as well as the target nucleic acids that correlate with fetal fraction are present in both the maternal and fetal genetic material and the assay is not specific for one over the other. In other words, the amounts of the target nucleic acids may not depend on the genetic makeup of the mother and fetus. In some embodiments, the maternal sample is cell-free DNA from maternal blood. Thus, in some embodiments, the technology described herein includes estimating a fetal fraction for a sample, wherein the fetal fraction is used to aid in the determination of whether the genetic data from a test subject is indicative of an aneuploidy.

### EXPERIMENTAL EXAMPLES

### Example 1

This example illustrates a preliminary investigation showing a proof of principle for the discovery of informative regions.

In this preliminary work, whole-genome shotgun sequencing data from approximately 12 k mixed fetal-maternal samples from male pregnancies and approximately 2.5 k mixed fetal-maternal samples from female pregnancies were analyzed. Read count data was analyzed in 1 kilobase (1 kb) sites, by adjusting for local sequence GC content and sequencing yield bias, and computing a fractional count for each site and sample. For each site, the samples were ordered by fractional read count and grouped (“binned”) into 50 equal sized groups (50 groups of samples each comprising approximately 2% of the samples, each group spanning a different range of fractional counts). This “binning” by fetal fraction was performed to allow more samples to be modelled, since the computational overhead of fitting a single regression model at all 1 kb sites across the genome was reduced from 14,500 unbinned observations to 50 binned observations. (An alternative method to reduce computational complexity is to model each site independently, as was done in Example 2.) For each group of samples, the read counts per site were piled up and normalized, and a group fetal fraction was estimated for the group using the median chromosome Y counts across samples in the group. This resulted in a matrix of normalized pile up counts, with a row for each site and a column for each group of samples, as well as a vector of per-group fetal fraction derived from the chr Y counts (by summing the counts from all chromosome Y sites and normalizing this by a total autosomal yield and a constant normalization factor) for the samples in the group. Ridge regression was applied to this data in order to identify sites where the site normalized pile-up counts for the groups of samples were predictive of the fetal fraction of the group. The significance of the regression coefficients was estimated using the Wald method. For each site, the effect size (regression coefficient) and its significance were recorded. Additionally, the Spearman rank correlation between the read counts at each and the fetal fraction for samples where this information was available (i.e. male fetus samples) was also calculated. The magnitude of this correlation and its significance were also recorded. The sites for which site-wide counts are significant negative predictors of fetal fraction (significant negative Spearman rank correlation/significant negative gain in Ridge regression) were identified as “negative FF predictors”. The sites for which site-wide counts are significant positive predictors of fetal fraction (significant positive Spearman rank correlation/significant positive gain in Ridge regression) were identified as “positive FF predictors”.

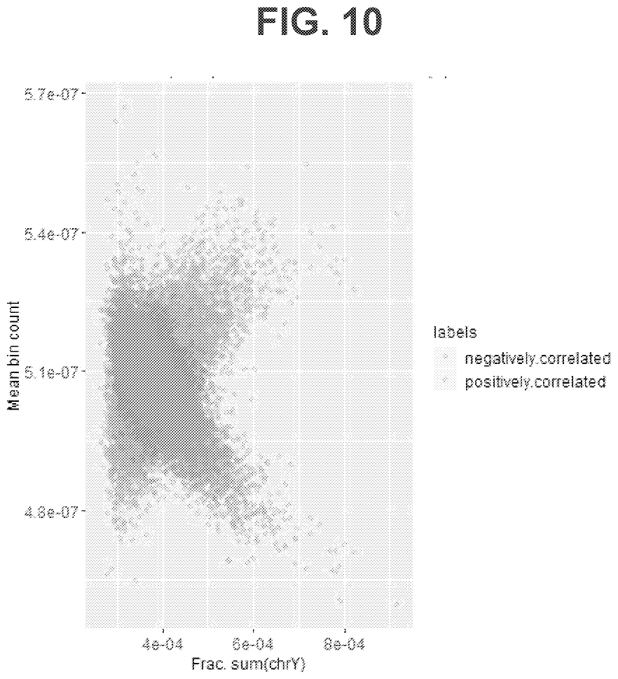

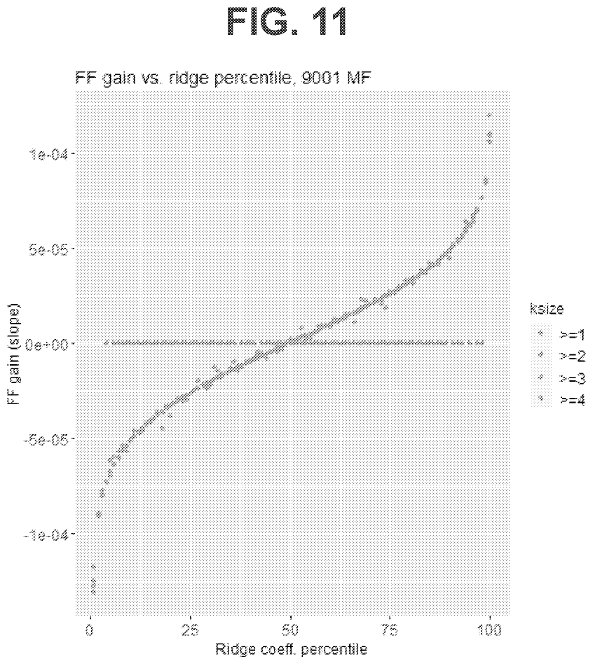

FIG. 10 shows the mean count per site as a function of the fetal fraction estimated from the chr Y counts for all sites that were identified as either significantly positively or negatively correlated with fetal fraction as determined by top effect size (ridge coefficient magnitude) percentiles. FIG. 11 shows the fetal fraction gain (slope of the Ridge regression model) in each of 100 groups of fitted Ridge regression models each comprising those site models with gains within a 1 percentile range of the distribution of gains estimates. The data show that both negative FF predictor sites and positive FF predictor sites can be identified. The data further showed that site-wide count distributions were similar for male and female fetuses. Finally, it was observed that predictive loci sometimes clusters in up to 3-mers of 1 kb each. This indicates that the sites likely reflect genuine biological effects associated with chromatin accessibility, and that as such informative regions do not necessarily align with arbitrarily defined regions.

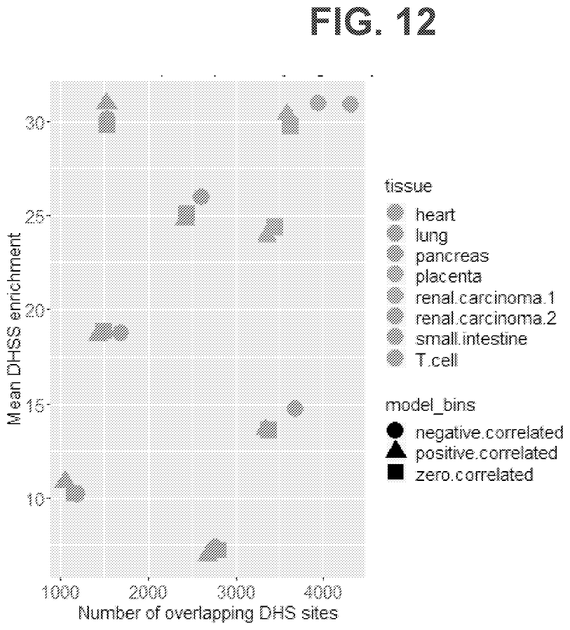

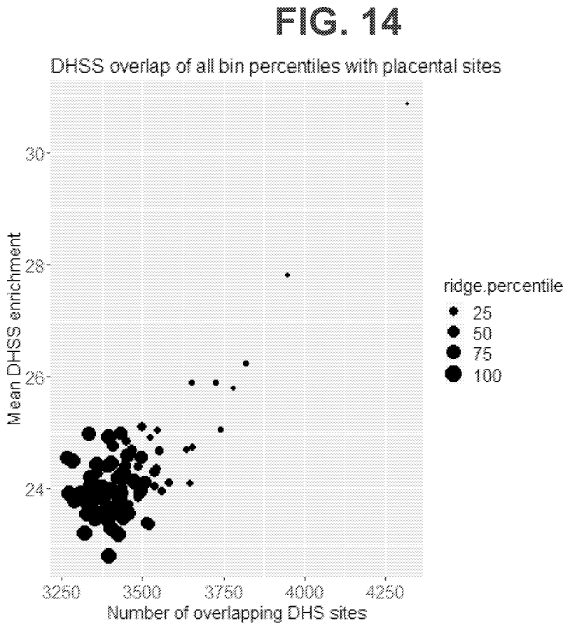

This was validated by looking at DNAse H sensitive (DHS) sites data in the ENCODE database. DHS sites are genomic regions that feature open chromatin. The overlap and enrichment (breadth and depth) of DHS sites within (i) neutral sites, (ii) negative predictor sites, and (iii) positive predictor sites, in a variety of including placenta (fetal tissue) and a variety of tissues that are assumed to be possible sources of maternal cfDNA was determined. The results of this analysis are shown on FIG. 12. This shows that DHS sites identified in placenta are particularly enriched in sites that are negatively correlated with fetal fraction. FIG. 13 shows the placental DHS sites enrichment for various groups of regions with Ridge gains within the indicated percentile groups of Ridge gains. The data again shows that highly negatively correlated FF sites are also strongly enriched in placental DHS sites. Thus, validation using DHS site data from the ENCODE database shows that the overlap and enrichment with DHS sites are highest for negative FF predictors in placenta, and that overlap and enrichment with DHS sites increase with increasing effect size (more significant negative predictors exhibit stronger overlap and stronger enrichment). This provides further confidence that sites with strong effect size (especially strong negative effect size) are likely to be good predictors of fetal fraction, because the underlying biological hypothesis (as explained in FIG. 2) is supported.

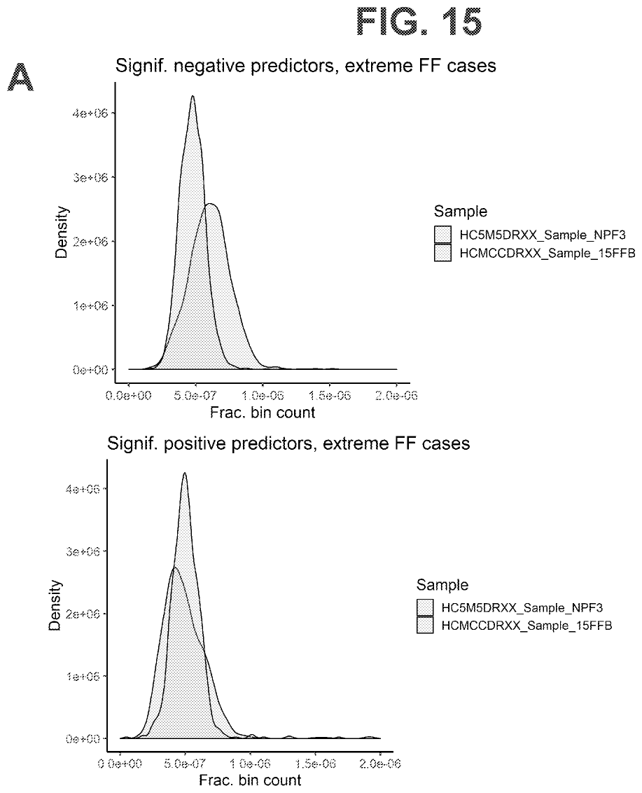

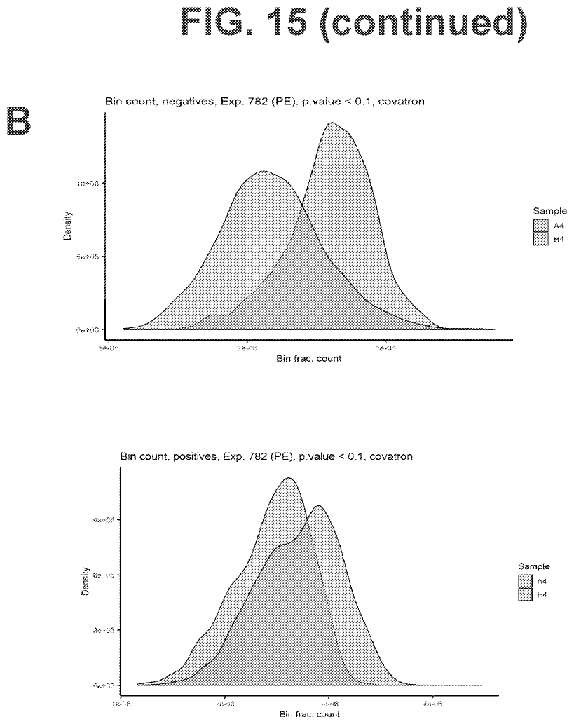

As a further validation, the inventors performed deep paired end sequencing of 15 new male fetus samples (1 kb coverage of approximately 250 to 300x), in order to verify that the signal for the candidate sites showed the correct differential trends for samples with significantly different fetal fraction. The results of this investigation are shown on FIG. 15, where each panel (A,B) shows the distribution of fractional counts per site for a respective pair of samples, shown separately for the negative predictor sites (top panel) and the positive predictor sites (bottom panel). The data shows that for each pair of samples, the distributions of counts per sites are significantly different between samples with different fetal fractions. The differences further have the expected direction:
sample A4 on FIG. 15B has a lower fetal fraction than sample H4 (approx. 5% for A4 vs approx. 15% for H4),
sample NPF3 on FIG. 15A has a lower fetal fraction than sample 15 ffB (approx. 5% for NPF3 vs approx. 15% for 15 ffB).

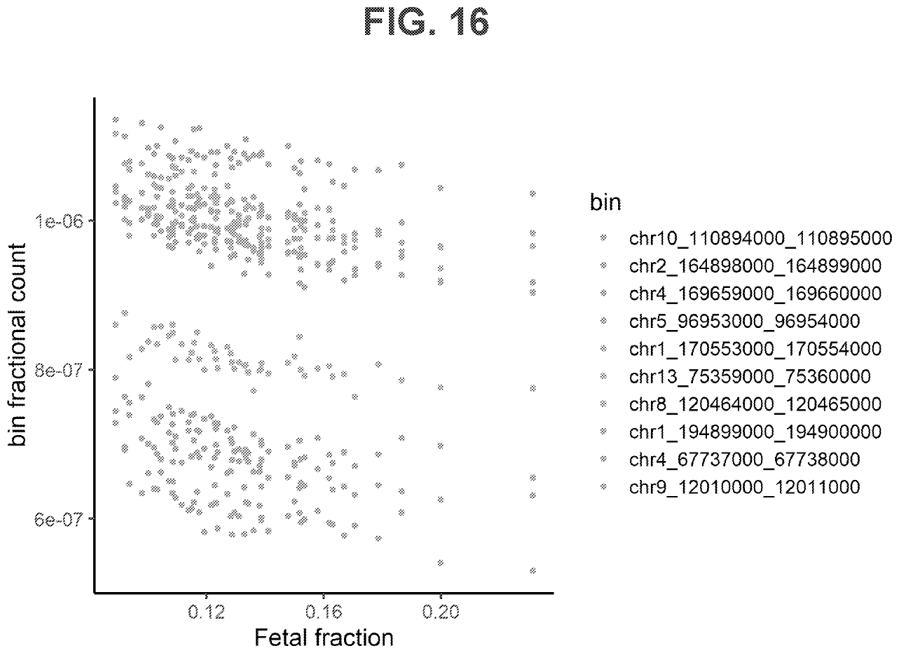

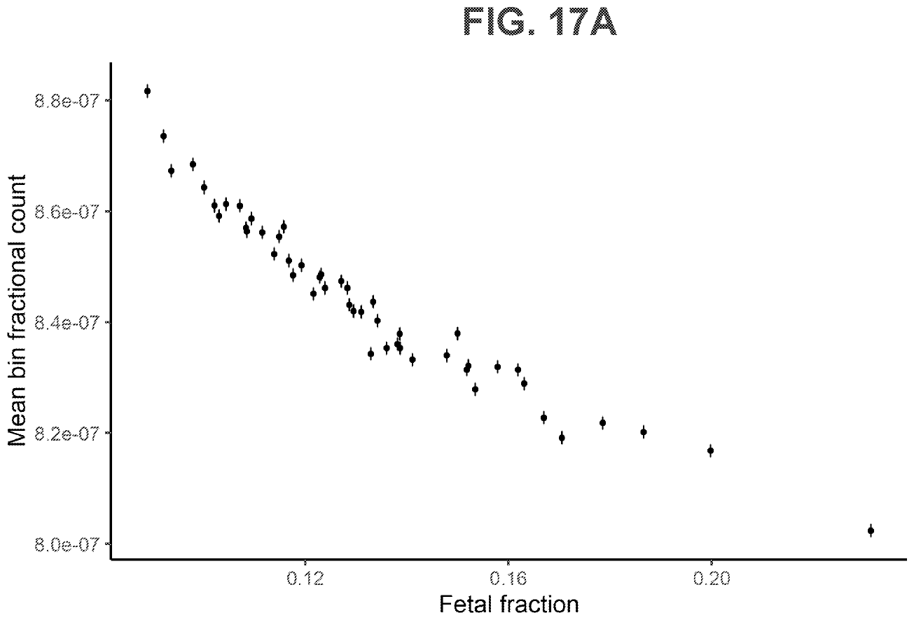

The inventors then investigated whether including additional data could improve the power of the bin discovery process. They therefore obtained sequencing data for an additional set of 15 kmale fetus samples, leading to a combined dataset of approximately 24 k samples. The analysis described above was repeated, i.e. summing read counts from groups of samples (approximately 470 samples per group) for each 1 kb site, then regressing this signal on the fetal fraction for the group, and recording the slope, intercept and goodness of fit. The goodness of fit may be used for selecting candidate predictor sites, for example to improve the signal to noise ratio. This data showed that using additional data does improve the power of discovery, and enabled the identification of many sites that correlate with fetal fraction. The signal for selected negative predictor sites is shown on FIG. 16, where each point represents the sum of counts for the site across all samples in a group, as a function of the fetal fraction for the group. The signal for the top negative predictors is shown on FIG. 17, where panel A shows the mean bin fractional count for the x sites that had the largest effect size for each of the 50 groups of samples, as a function of the mean FF for the group of samples. FIG. 17B shows the coefficient of variation of the signal with mean on FIG. 17A, for each of the 50 groups of samples, as a function of the mean FF for the group of samples. The error bars on FIGS. 17A-B show the standard error of the mean for the values shown. This data demonstrated that the intercept for site varied (see FIG. 16), as did the gain (data not shown), and the regression goodness of fit (data not shown). However, the data showed that top negative predictors were associated with counts that have relatively low variability in each of the respective groups of fetal fractions, indicating that useful estimates of fetal fraction may be obtained by measuring counts for these sites.

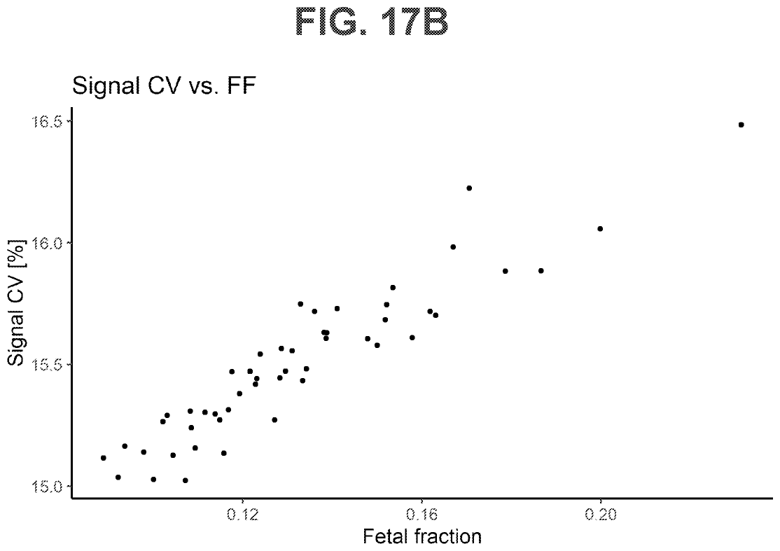

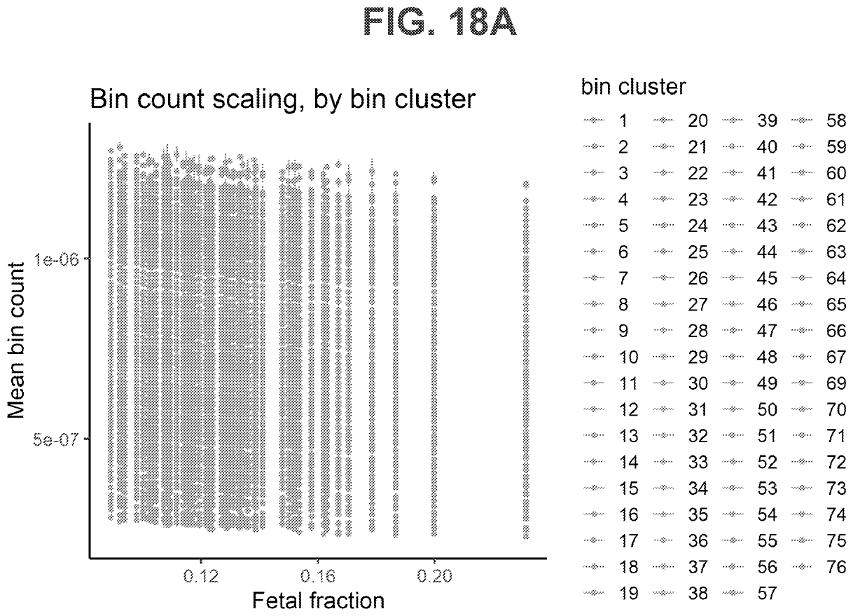

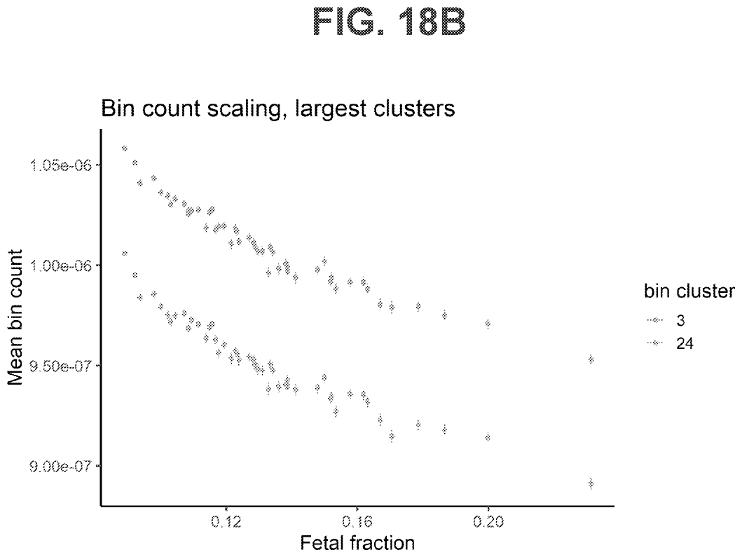

The inventors further explored whether the sites could be grouped into clusters, where the signal from clusters would be used to estimate the fetal fraction, instead of the signal from individual sites. The inventors hypothesized that this approach may result in more reliable estimates. Thus, all negative FF predictor sites were clustered using the regression intercept as a feature on which clustering was based, using hierarchical clustering. A total of 76 clusters were identified and the data for each of these clusters are shown on FIG. 18A. The data for the two largest clusters are shown on FIG. 18B.

### Example 2

This example illustrates a process for the discovery of informative regions.

Using a proprietary data set of cfDNA samples from pregnant woman sequenced on the Illumina platform, the inventors performed analyses to identify genomic regions that were fetal responsive. In particular, they investigated the sample data set for genomic sites with either increasing or decreasing read coverage observed with increasing fetal fraction percentages. This analysis resulted in the discovery of regions of the genome that appeared to be fetal responsive.

### Experimental Approach

Approximately 26,000 de-identified DNA samples obtained from pregnant women were converted into Illumina sequencing libraries using a TruSeq NANO DNA LT Kit with either barcode set A or set B (Catalog Number: FC-121-4001 (A) and FC-121-4002 (B)). Following library preparation, the samples were sequenced on an Illumina HiSeq 2500 and the resulting reads were aligned to the genome and counted. The resulting count data were used in conjunction with the algorithms and tools described below to identify sites of the genome enriched in either maternal or fetal origin DNA.

### General Mathematical Framework

A mathematical framework to identify genomic regions that are indicative of fetal fraction was developed. This is explained below.

Let Y_{i,j}, be the count of molecules assayed from a cfDNA sample of from genomic site i of known ploidy and sample j. This count is a mixture of molecules of mixed maternal and fetal origin and is modelled as a homogenous counting process with expectation:

$$E [ {Y}_{i , j} ] = {μ}_{i , j} = {τ}_{j} {λ}_{i , j}$$

where τⱼ≥0 is the sample-specific assay yield across the genomic sites of known ploidy and λ_{i,j}≥0 is a sample- and site-specific “enrichment” factor that is characteristic to the assay.

For much of the genome, λ_{i,j} depends only on intrinsic characteristics of each genomic site like the nucleotide sequence, GC content, and ability to uniquely determine the location within the genome. In cfDNA mixtures with differential maternal and fetal enrichment λ_{i,j} can differ across samples as a function of the fraction of molecular counts of maternal versus fetal origin. It can be assumed that λ_{i,j} is a weighted average:

$$\begin{matrix}{λ}_{i , j} = ( 1 - {f}_{j} ) {λ}_{i , m} + {f}_{j} {λ}_{i , f} \\ = {λ}_{i , m} + {f}_{j} ( {λ}_{i , f} - {λ}_{i , m} )\end{matrix}$$

where fⱼ is the fetal fraction of cfDNA sample j with values in [0,1], 1−fⱼ is the maternal fraction, λ_{i,m}≥0 is the genomic site-specific maternal enrichment factor, and λ_{i,f}≥0 is the genomic site-specific fetal enrichment factor.

As the expected value of the sum of random variables is equal to the sum of their individual expected values (a property referred to as the linearity property of expectations), the expected value can be expressed and interpreted in several equivalent ways:

$$\begin{matrix}E [ {Y}_{i , j} ] = {μ}_{i , j} & ( Scalar mean ) \\ = {τ}_{j} {λ}_{i , j} & ( product of yield \& enrichment ) \\ = {τ}_{j} ( ( 1 - {f}_{j} ) {λ}_{i , m} + {f}_{j} {λ}_{i , f} ) & ( weighted average ) \\ = {τ}_{j} {λ}_{i , m} + {f}_{j} {τ}_{j} ( {λ}_{i , f} - {λ}_{i , m} ) & ( line with slope ∝ {f}_{j} )\end{matrix}$$

For samples of known fetal fraction, finding the sites with statistically significant maternal or fetal enrichment bias is equivalent to testing a hypothesis at each site:

$$\begin{matrix}{H}_{0} : {λ}_{i , f} = {λ}_{i , m} or {λ}_{i , f} - {λ}_{i , m} = 0 \\ {H}_{1} : {λ}_{i , f} \neq {λ}_{i , m} or {λ}_{i , f} - {λ}_{i , m} \neq 0\end{matrix}$$

The formulations above describe tests with a single degree of freedom, which are advantageously more powerful than equivalent tests formulated with multiple degrees of freedom.

Molecular counts Y_{i,j}, can be modelled using a variety of discrete homogeneous processes. In practice, Poisson or negative binomial processes are sensible choices, depending on whether over dispersion is suspected or observed in the cfDNA assay. In particular, the Poisson distribution is commonly used to model count data, and has a single free parameter (i.e., the variance is not adjusted independently of the mean). Thus, where over dispersion is suspected or observed (i.e. the variability in the data is greater than would be expected under the best fitting Poisson distribution), models with additional free parameters that are suitable for modelling count data may be preferred. For example, a Poisson mixture model like the negative binomial distribution may be used, in which the mean of the Poisson distribution is modelled as a random variable drawn from the gamma distribution. Alternatively, the distribution of Y_{i,j}, can be approximated by a Normal distribution when the mean is sufficiently large or after a suitable transformation (such as e.g., a log transformation). Indeed, for sufficiently large values of the Poisson distribution parameter λ (such as e.g., ζ>1000), the Normal distribution with mean k and variance k may be used as a suitable approximation of the Poisson distribution. As the skilled person understands, whether the approximation is acceptable depends on the circumstances. Additionally, the Normal distribution may also be a good approximation of a Poisson distribution if an appropriate continuity correction is performed (i.e. if P(X≤x), where x is a non-negative integer, is replaced by P(X≤x+0.5)) and the value of λ is not too small (e.g., λ>10). A suitable transformation may be one that, when applied to the count data, results in approximately normally distributed data. Whether a normal distribution is a good fit for a particular data set can be estimated using a normality test, as known in the art. The above approach describes how generalized linear models (where the distribution of the dependent variable can follow any distribution in the exponential family of distributions) can be used to infer whether a site is informative. However, other methods can be used to accomplish this task. For example, multiple non-parametric methods (e.g., non-parametric regression), quasi-likelihood methods and deep learning approaches (e.g., neural networks or some deep learning techniques can be viewed as an application of non-parametric regression, e.g., decision trees like CART and support vector machines) may be utilized to similarly infer which sites are informative. Examples using a Poisson distribution, a negative binomial distribution and a Gaussian distribution, respectively, as the assumed distribution for read counts will be described in detail below.

### Poisson-Distributed Counts

Let Y be an i×j matrix of Poisson distributed discrete random variables with values y_{i,j}, that represents the distribution of molecular counts observed at genomic site i in sample j:

$${Y}_{i , j} ∼ Poisson ( {μ}_{i , j} ) where {μ}_{i , j} = {τ}_{j} {λ}_{i , j} = {τ}_{j} ( ( 1 - {f}_{j} ) {λ}_{i , m} + {f}_{j} {λ}_{i , f} )$$

is a function of the following 2(n+m) parameters:

| Parameter | Description | Cardinality |
|---|---|---|
| λ_{i, m} | Expected molecular count per unit of | n |
| | sequencing for a pure maternal sample | |
| | at site i (per-site maternal count) | |
| λ_{i, f} | Expected molecular count per unit of | n |
| | sequencing for a pure fetal sample | |
| | at site i (per-site fetal count) | |
| τⱼ | Total yield (observed molecular count) | m |
| | at all sites of known ploidy for sample | |
| | j (per-sample yield) | |
| fⱼ | Fraction of molecules of fetal origin | m |
| | for sample j (per-sample fetal fraction) | |

where m is the number of samples and n is the number of sites considered.

The conditional expectation and variance of Yⱼ(molecular counts at site i for sample j), is:

$$E [ {Y}_{i , j} \vert {μ}_{i , j} ] = Var [ {Y}_{i , j} \vert {μ}_{i , j} ] = {μ}_{i , j} = {τ}_{j} {λ}_{i , j}$$

The conditional probability mass function is:

$$f ( {Y}_{i , j} = {y}_{i , j} \vert {μ}_{i , j} ) = \frac{{μ}_{i , j}^{{y}_{i , j}} {e}^{- {μ}_{i , j}}}{{y}_{i , j} !} , for {y}_{i , j} = 0 , 1 , 2 , …$$

The conditional log likelihood is:

$$\begin{matrix}log ℒ ( {Y}_{i , j} = {y}_{i , j} \vert {μ}_{i , j} ) = log f ( {y}_{i , j} \vert {μ}_{i , j} ) \\ = {y}_{i , j} log ( {μ}_{i , j} ) - {μ}_{i , j} - log ( {y}_{i , j} ! )\end{matrix}$$

### Negative-Binomial Distribution of Counts

Let Y be an i×j matrix of negative binomially distributed discrete random variables with values y_{i,j}, that represents the distribution of molecular counts observed at genomic site i in sample j:

$${Y}_{i , j} ∼ NB 2 ( {μ}_{i , j} , {α}_{i} )$$

$$where {μ}_{i , j} = {τ}_{j} {λ}_{i , j} = {τ}_{j} ( ( 1 - {f}_{j} ) {λ}_{i , m} + {f}_{j} {λ}_{i , f} )$$

is a function of the following 3n+2 m parameters:

| Parameter | Description | Cardinality |
|---|---|---|
| λ_{i, m} | Expected molecular count per unit of | n |
| | sequencing for a pure maternal sample | |
| | at site i (per-site maternal count) | |
| λ_{i, m} | Expected molecular count per unit of | n |
| | sequencing for a pure fetal sample | |
| | at site i (per-site fetal count) | |
| αᵢ | Overdispersion parameter at site i | n |
| | (per-site dispersion) | |
| τⱼ | Total yield (observed molecular count) | m |
| | at all sites of known ploidy for sample | |
| | j (per-sample yield) | |
| fⱼ | Fraction of molecules of fetal origin | m |
| | for sample j (per-sample fetal fraction) | |

where m is the number of samples and n is the number of sites considered.

The conditional expectation of Y_{i,j} (molecular counts at site i for sample j), is:

$$E [ {Y}_{i , j} \vert {μ}_{i , j} ] = {μ}_{i , j} = {τ}_{j} {λ}_{i , j}$$

The conditional variance of Yⱼ(molecular counts at site i for sample j), is:

$$Var [ {Y}_{i , j} \vert {μ}_{i , j} ] = {μ}_{i , j} + {α}_{i} {μ}_{i , j}^{2}$$

The conditional probability mass function is:

$$f ( {y}_{i , j} \vert {μ}_{i , j} , {α}_{i} ) = \frac{Γ ( {y}_{i , j} + {r}_{i} )}{Γ ( {y}_{i , j} + 1 ) Γ ( {r}_{i} )} {{p}_{i , j}^{{r}_{i}} ( 1 - {p}_{i , j} )}^{{y}_{i , j}} , for {y}_{i , j} = 0 , 1 , 2 , … .$$

where rᵢ=1/αᵢ, p_{i,j}=1/(1+αᵢμ_{i,j}) and Γ(z)=∫₀^{∞}z^{z-1}e^{-x}dx is the gamma function.

The conditional log likelihood is:

$$\begin{matrix}log ℒ ( {y}_{i , j} \vert {μ}_{i , j} , {α}_{i} ) = log f ( {y}_{i , j} \vert {μ}_{i , j} , {α}_{i} ) \\ = {y}_{i , j} log ( 1 - {p}_{i , j} ) + {r}_{i} log ( {p}_{i , j} ) + \\ log Γ ( {y}_{i , j} + {r}_{i} ) - log Γ ( {y}_{i , j} + 1 ) - log Γ ( {r}_{i} )\end{matrix}$$

### Gaussian Distribution of Counts

Let Y be an i×j matrix of normally distributed random variables with values y_{i,j}, that represents the distribution of molecular counts observed at genomic site i in sample j:

$${Y}_{i , j} ∼ N ( {μ}_{i , j} , {σ}_{i}^{2} )$$

$$where {μ}_{i , j} = {τ}_{j} {λ}_{i , j} = {τ}_{j} ( ( 1 - {f}_{j} ) {λ}_{i , m} + {f}_{j} {λ}_{i , f} )$$

is a function of the following 3n+2 m parameters:

| Parameter | Description | Cardinality |
|---|---|---|
| λ_{i, m} | Expected molecular count per unit of | n |
| | sequencing for a pure maternal sample | |
| | at site i (per-site maternal count) | |
| λ_{i, f} | Expected molecular count per unit of | n |
| | sequencing for a pure fetal sample | |
| | at site i (per-site fetal count) | |
| σᵢ² | Variance of counts at site i | n |
| | (per-site variance) | |
| τⱼ | Total yield (observed molecular count) | m |
| | at all sites of known ploidy for sample | |
| | j (per-sample yield) | |
| fⱼ | Fraction of molecules of fetal origin | m |
| | for sample j (per-sample fetal fraction) | |

where m is the number of samples and n is the number of sites considered.

The conditional expectation of Y_{i,j} (molecular counts at site i for sample j), is:

$$E [ {Y}_{i , j} \vert {μ}_{i , j} ] = {μ}_{i , j} = {τ}_{j} {λ}_{i , j}$$

The conditional variance of Y_{i,j} (molecular counts at site i for sample j), is:

$$Var [ {Y}_{i , j} \vert {μ}_{i , j} ] = {σ}_{i}^{2}$$

The conditional probability density function is:

$$f ( {y}_{i , j} \vert {μ}_{i , j} , {σ}_{i}^{2} ) = \frac{1}{{σ}_{i} \sqrt{2 π}} {e}^{- \frac{1}{2} ( \frac{{y}_{i , j} - {μ}_{i , j}}{{σ}_{i}} )} .$$

The conditional log likelihood is:

$$\begin{matrix}log ℒ ( {y}_{i , j} \vert {μ}_{i , j} , {σ}_{i}^{2} ) = log f ( {y}_{i , j} \vert {μ}_{i , j} , {σ}_{i}^{2} ) \\ = - log ( {σ}_{i} \sqrt{2 π} ) - \frac{1}{2} {( \frac{{y}_{i , j} - {μ}_{i , j}}{{σ}_{i}} )}^{2}\end{matrix}$$

### Methods for Discovery of Informative Genomic Sites for Estimation of Fetal Fraction

At each genomic location, the above likelihood (e.g., the Poisson conditional log likelihood, or likelihood defined according to any other chosen distribution, or an approximation of any of the former) can be maximized using direct numerical optimization of the likelihood function, in order to estimate the fetal and maternal enrichment parameters (λ_{i,m} and λ_{i,f}) given training data that comprises individuals of known fetal fraction (1−fⱼ, fⱼ) and molecular count yield (τⱼ). For example, methods like gradient descent, iteratively reweighted least squares, etc. may be used. Statistical significance can be obtained from multiple methods, including Wald tests, score tests, likelihood ratio tests, etc. Variants of these methods utilizing non-parametric and deep learning approaches to building predictors of fetal or maternal enrichment are also possible embodiments. For example, quasi-likelihood estimation may be used instead of maximum likelihood estimation, non-parametric regression may be used instead of generalized linear model regression, and machine learning algorithms (e.g., k-nearest neighbors, decision trees, support vector machines, neural networks, etc.) may be used to build predictors of fetal or maternal enrichment.

Methods Estimation of Fetal Fraction from Count Data

For counts from a given sample, the above likelihood (e.g., the Poisson conditional log likelihood, or likelihood defined according to any other chosen distribution, or an approximation of any of the former) can be maximized using direct numerical optimization of the likelihood function, in order to estimate the fetal and maternal fraction parameters given the fetal and maternal enrichment parameters (λ_{i,m} and λ_{i,f}), estimated from training data and the molecular count yield (τⱼ). For example, methods like gradient descent, iteratively reweighted least squares, etc. may be used. Statistical significance can be obtained from multiple methods, including Wald tests, score tests, likelihood ratio tests, etc. Variants of these methods utilizing non-parametric and deep learning approaches to building predictors of fetal fraction are also possible embodiments. For example, quasi-likelihood estimation may be used instead of maximum likelihood estimation, non-parametric regression may be used instead of generalized linear model regression, and machine learning algorithms (e.g., k-nearest neighbors, decision trees, support vector machines, neural networks, etc.) may be used to build predictors of fetal or maternal enrichment.

### Results

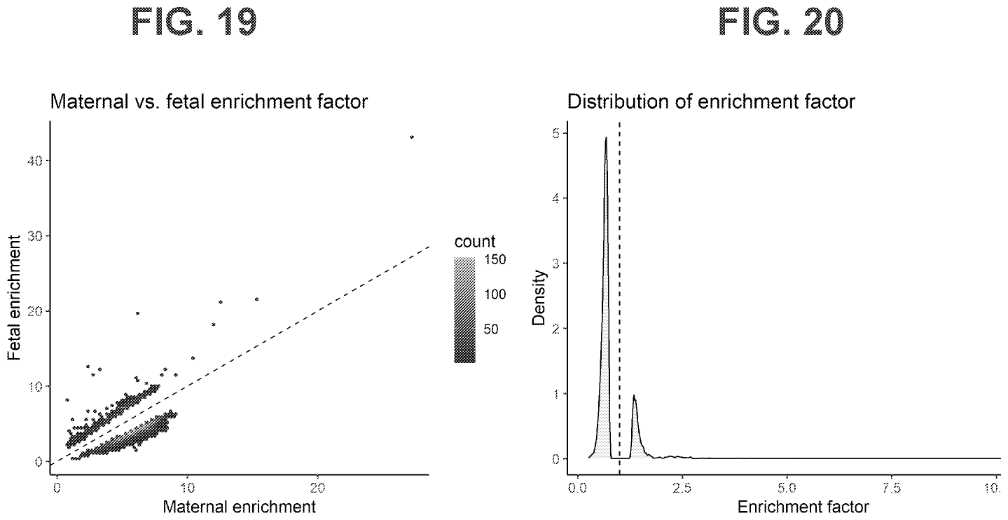

In this example, a Poisson model as described above was fitted to genomic read count data for m=26,500 samples, where the counts were aggregated for sites defined as n=2,757,964 contiguous regions of 1 kbases distributed along the human reference genome. Using this model, at total of 3551 sites were identified as being associated with a significantly different maternal and fetal enrichment factors (i.e., H₁ of the hypothesis test provided above was identified to be true). FIG. 19 shows the maternal vs. fetal enrichment factors for the 5000 most significant and highest-effect-size genomic sites identified in the discovery process. The darkness of each point indicates the number of loci that have a specific combination of maternal and fetal enrichment factors. The dashed line is the identity line (i.e. λ_{i,m}=λ_{i,f}). FIG. 20 shows the distribution of the enrichment ratio (defined as fetal/maternal enrichment, i.e. λ_{i,f}/λ_{i,m}). The vertical dashed line denotes the “neutral” decision boundary (enrichment ratio=1). As shown on FIG. 30, the negative predictor sites with the highest effect size had an effect size of approximately 30% (i.e., an enrichment ratio

$$( \frac{{λ}_{f}}{{λ}_{m}} ) ,$$

of approximately 0.7). Many sites had effect sizes between 10 and 30%, particularly between 10 and 20%.

### Statistical Power Calculations

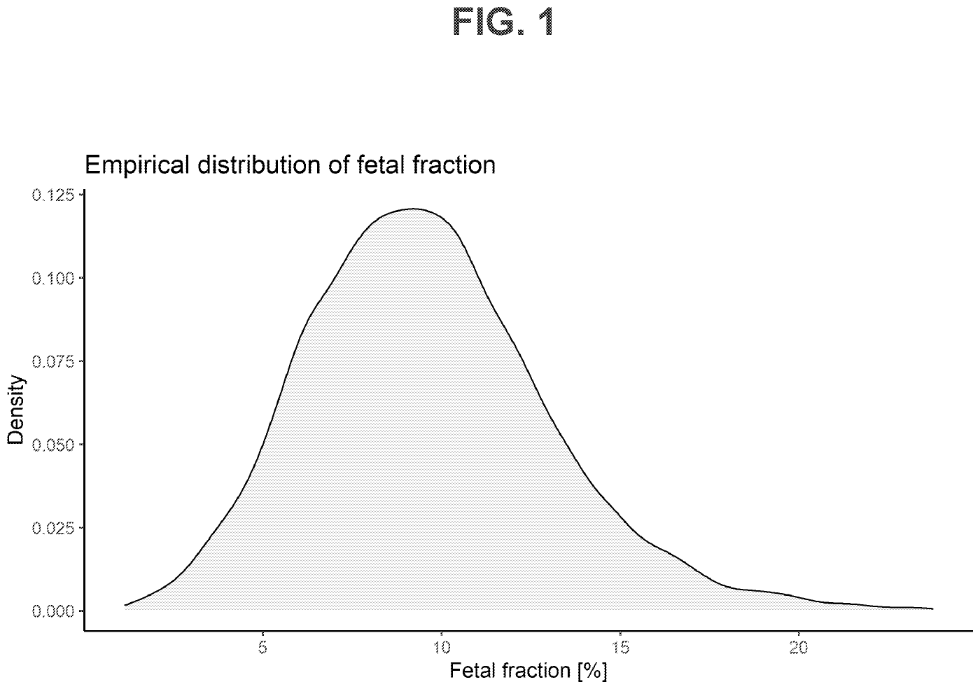

To determine how many genomic sites should be targeted for calling fetal fraction, the inventors performed statistical power calculations. In these calculations, they assumed that site targeting (the capture of a particular genomic region using e.g., molecular inversion probes as described in WO 2019/195346 to Sekedat, et al. titled, “Methods, Systems, and Compositions for Counting Nucleic Acids,” and WO 2020/206170 to Sekedat, et al. also titled, “Methods, Systems, and Compositions for Counting Nucleic Acid Molecules) can be described by a simple binomial process, with the target capture probability of 0.9 (probe capture efficiency=90%). Further, to mimic sampling volume specifications, they assumed that for each targeted site, 2900 genome copies are present in the “sample”. For each such “sample”, a fetal fraction was drawn from the in-house empirical distribution of fetal fractions shown on FIG. 1.

To simplify matters, statistical power calculations were performed by assuming that all sites have the same level of enrichment. To inform about “typical” levels, FIGS. 19 and 20 show the maternal enrichment, fetal enrichment, and the enrichment ratio (λₘ, λ_{f}, and

$$\frac{{λ}_{f}}{{λ}_{m}} ,$$

respectively) for 5000 most statistically significant sites identified by the above discovery process. Inspecting FIG. 20, we see that most of the top statistically significant sites have higher maternal enrichment (implying the molecular count at these sites decreases with increasing fetal fraction). This is consistent with the chromatin accessibility hypothesis of the fetal cfDNA abundance in plasma, i.e., that genomic regions that are in open chromatin in the fetal DNA and not in adult maternal DNA are more likely to reliably correlate with fetal fraction. In addition, for most of the loci with preferential maternal enrichment (enrichment ratio <1), the enrichment ratio is typically significantly lower than 1. Thus, to determine how the enrichment ratio affects the ability to call fetal fraction at different numbers of targeted loci, in the following calculations the fetal enrichment was set to 1, and maternal enrichments that would yield enrichment ratios of 0.8 and 0.9 were considered.

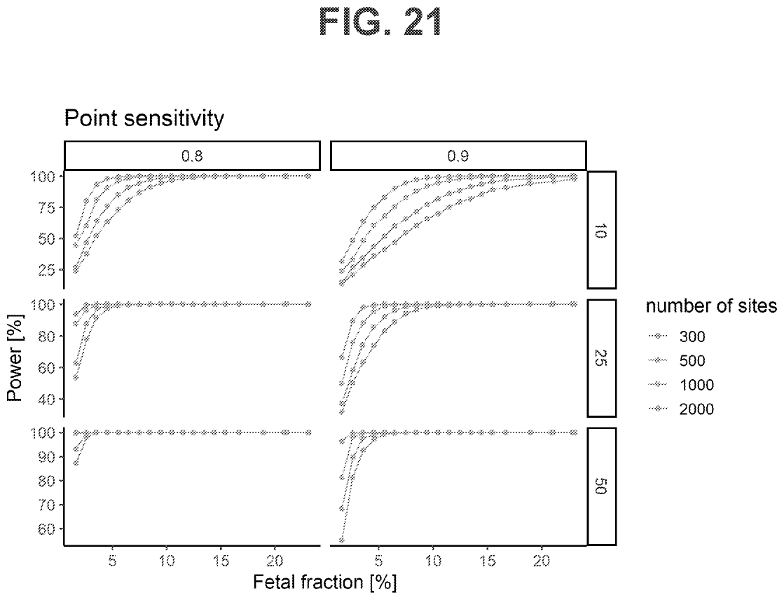

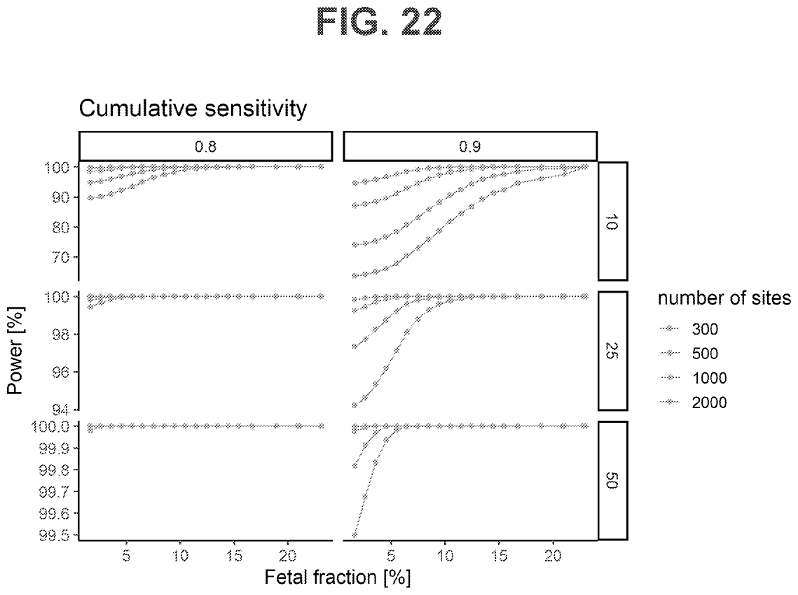

FIG. 21 shows the results for the point sensitivity of fetal fraction detection vs. the fetal fraction, for different numbers of targeted loci (300, 500, 1000, and 2000) and different enrichment ratios (columns: 0.8 and 0.9). For a given fetal fraction, point sensitivity is the fraction of “hits”, where the predicted fetal fraction was considered a “hit” if its value was within a predefined relative error of the actual fetal fraction. (Different values of relative errors were considered, rows in FIG. 21: 10%, 25%, 50%.) FIG. 22 shows the results for the cumulative sensitivity of fetal fraction detection vs. the fetal fraction, for different numbers of targeted loci (300, 500, 1000, and 2000) and different enrichment ratios (columns: 0.8 and 0.9). For a given fetal fraction, cumulative sensitivity is the overall sensitivity to detect fetal fractions in samples with equal or higher fetal fraction (note that the cumulative sensitivity is weighted by the distribution of fetal fraction). Again, relative error was used for quantification of sensitivity, and different relative errors were considered (rows in FIG. 22: 10%, 25%, 50%).

The results of this statistical power analysis suggest that, under the above assumptions (1-Kbp-wide sites, enrichment ratio ˜0.8-0.9, a simple binomial process describing target capture) very accurate estimation of fetal fraction is possible even with as few as 1000 target loci. However, the results obtained using this statistical power analysis represent theoretical bounds (which are often non-achievable in practice, as they do not take into account over dispersion due to biological variability or variability introduced by the experimental process used to obtain the count data). Indeed, actual sensitivity is likely to be lower owing to the imperfection of the experimental apparatus. As such, the actual number of sites that should be targeted would beneficially be higher, with exact numbers depending on the level of certainty in the fetal fraction estimate that is desired, as well as the level of noise associated with the experimental platform used, and the effect size associated with the particular sites chosen.

### Example 3

This example illustrates the design of molecular inversion probes for the capture and generation of molecular counts for informative and uninformative regions, and the validation of a method for estimating fetal fraction using molecular counts from target sequences.

To develop an assay based on the observations in Examples 2 and 3, molecular inversion probes (MIPs) were designed to target genomic regions that showed fetal responsiveness. Since it was observed that the magnitude of the signal change varied between genomic locations, with the best effective size reflecting an approximate 30% change, the genomic regions with the highest effect size and also the highest P value in the original data set (Example 2) were targeted.

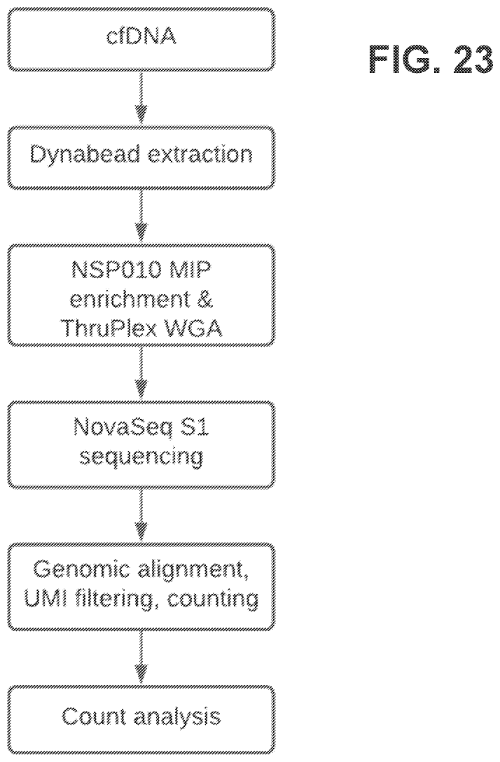

To observe the fetal responsive genomic locations using cfDNA, experiments were performed on whole genome amplified (WGA) cfDNA using the protocol provided below and illustrated on FIG. 23.

The results suggested that the fetal responsive bins were observed in cfDNA samples derived from patient plasma. In addition, the effect size was similar and the same direction as previous experiments. Some noise was observed, likely at least in part due to the whole genome amplification step.

Similar cfDNA samples with identical fetal fractions, as determined by another assay, were then pooled and analyzed using the same MIPs. This approach also demonstrated that the genomic sites chosen as fetal responsive had a good effect size and were statistically significantly associated with fetal fraction.

### Design of Molecular Inversion Probes

A set of MIPs targeting approximately 4400 genomic sites that were identified as the strongest negative FF predictors using the analysis in Example 2 were designed. According to the power analysis results (Example 2), a few thousand sites are sufficient for achieving a good prediction performance. Thus, ˜4,400 probes were selected as top candidates (from a total pool of ˜36,000 probes targeting 3,270 sites); those probes targeted the most statistically significant and highest-effect sites that were identified by the mathematical model. Each probe had a genomic footprint of approximately 80-120 bases. An average of 11 MIPs targeting each 1 kb site were included.

### DNA Extraction and Generation of Molecular Counts

1. cfDNA was isolated from plasma as previously described (see FIG. 23, step “Dynabead extraction”)
2. Use a WGA approach to amplify the amount of cfDNA available to use for the remainder of the assay. WGA approaches such as Takara's ThruPLEX Tag-Seq HV (www.takarabio.com/products/next-generation-sequencing/dna-seq/dna-seq-for-ffpe-and-cell-free-dna) (see www<dot>takarabio<dot>com/products/next-generation-sequencing/dna-seq/picoplex-gold-for-single-cells?catalog=R300669) (see FIG. 23, step “ThruPlex WGA”).
3. Target Capture Step (see FIG. 23, step “NSP010 MIP enrichment”)

| a. | Target Capture Reagent Recipe | (x1) |
|---|---|---|
| b. | 10x Ampligase Buffer | 2 |
| c. | 17 uM pool of fetal site-specific | 1 |
| | Molecular Inversion probes | |
| d. | Amplified cfDNA (100-250 nanograms) | 17 |
| e. | Total Volume | 20 |

| | |
|---|---|
| f. | Add the appropriate dilution of gDNA, uM MIP pool, |
| | and 10x Ampligase buffer to each well. |

g. Seal plate. Vortex to mix, then spin down. Place in PCR machine and run the below protocol:

| | | |
|---|---|---|
| i. | Capture | Program |
| ii. | 98 C. | 3 min |
| iii. | touchdown | ~90 min (2 mins/degree) -Set ramp |
| | | speed to 20% for TD temps |
| iv. | 56 C. | 120 min |

h. Following 56 C incubation, remove plate from thermocycler, spin down, and remove plate seal
i. Set up and add the extension/ligation mix to each well per the table below:
4. Extension/Ligation step

| a. | Extension & Ligation Reagent Recipe | (x1) |
|---|---|---|
| i. | Capture Sample from step 2i | 20 |
| ii. | 10 mM dNTP | 0.6 |
| iii. | 100X NAD | 0.8 |
| iv. | 5M Betaine | 3 |
| v. | 10X Ampligase Buff | 2 |
| vi. | Ampligase, 5 U/ul | 2 |
| vii. | Phusion Pol HF, 2 U/ul | 0.5 |
| viii. | Nuclease free water | 11.1 |
| | Total Volume | 40 |

b. Seal plate. Vortex to mix, then spin down. Place in PCR machine and run the below protocol:
c. Extension/Ligation program

| | | |
|---|---|---|
| i. | 56 C. | 60 min |
| ii. | 72 C. | 20 min |
| iii. | 4 C. | hold |

d. Following step down, remove plate from thermocycler, spin down, and remove plate seal
e. Set up and add the exonuclease digest mix to each well per the table below:
5. Exonuclease step

| a. | Exonuclease Digest Reagent | (x1) |
|---|---|---|
| b. | Ligation Reaction from step 3H | 40 |
| c. | 10x NEB Buffer 1.1 | 5 |
| d. | Exonuclease I, 20 U/uL | 2 |
| e. | Exonuclease III, 100 U/uL | 2 |
| f. | Nuclease free water | 1 |
| g. | Total Volume | 50 |

h. Seal plate. Vortex to mix, then spin down. Place in a PCR machine and run the below protocol:
i. Exonuclease program

| | | |
|---|---|---|
| i. | 37 C. | 55 min |
| ii. | 90 C. | 40 min |
| iii. | 4 C. | hold |

6. Amplification Step-PCR based approach to creating Illumina Sequencing Libraries

| a. | PCR Reagent | (x1) |
|---|---|---|
| b. | Exo product from step 4i | 20 |
| c. | 5X Phusion HF buffer | 10 |
| d. | 10 mM dNTPs | 1 |
| e. | Phusion Pol HS, 2 U/ul | 1 |
| f. | FW primer (100 uM) | 0.25 |
| g. | Universal primers (Rev, 5 uM) | 5 |
| h. | Nuclease free water | 12.75 |
| | Total Volume | 50 |

i. add the appropriate samples, index primers, and PCR MM to each well.
j. Seal plate. Vortex to mix, then spin down. Place in BioRad qPCR system and run the below protocol:
k. Amplification program

| | | |
|---|---|---|
| i. | 98 C. | 3 min |
| ii. | 98 C. | 10 sec |
| iii. | 65 C. | 20 sec |
| iv. | 72 C. | 30 sec |

v. Repeat step ii-iv 17 times, repeat steps ii-iv for a total of 17 amplification cycles

| | | |
|---|---|---|
| vi. | 72 C. | 5 min |
| vii. | 4 C. | hold |

7. Purification Step using AMPure XP beads from Beckman Coulter (see www<dot>77eckman<dot>com/reagents/genomic/cleanup-and-size-selection/pcr)
   a. Bead ratio: 1.5 Total sample volume: 50 Volume beads to add: 75
   b. Remove AMPure beads from 4 C and equilibrate to RT for ˜½ an hour
   c. Add AMPure beads to each sample and mix
   d. Incubate at RT for 5 min
   e. Place on magnet and allow to separate for at least 2 min
   f. Remove supernatant and discard
   g. Wash the beads with 180 ul of 80% EtOH two times (resuspend with pipette)
   h. Allow beads to air dry for 3 min on the magnet, look for dryness
   i. Remove from magnet and add 50 ul of EB to the beads to resuspend
   j. Incubate at RT for 5 min
   k. Place tube on magnet and allow to separate
   l. Remove supernatant and dispense into new plate
8. Sequence libraries on Illumina Sequencing platforms (See step “NovaSeq S1 sequencing”, FIG. 23).

### Data Analysis

Sequencing data was analysed by aligning reads to the human reference genome (see FIG. 21, step “genomic alignment”), removing duplicate and error reads (i.e., filtering for unique reads) using unique molecular identifiers included in the sequencing library (see FIG. 23, step “UMI filtering”), and counted (see FIG. 23, step “counting”). In particular, the total number of reads mapping to each targeted site was obtained, for each sample. Alignment was performed using bowtie2 (see world wide web at bowtie-bio.sourceforge.net/bowtie2/index.shtml), using reference genome GRCh38. Adapter trimming was performed using cutadapt (https://cutadapt.readthedocs.io/en/stable/), UMI processing was performed using umitools (https://github.com/weng-lab/umitools), and read counting was done using an in-house program.

### Results

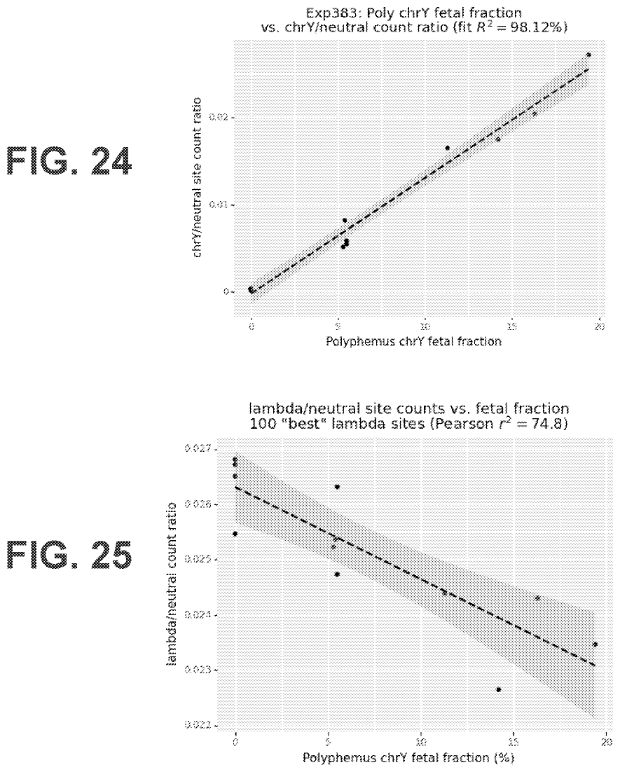

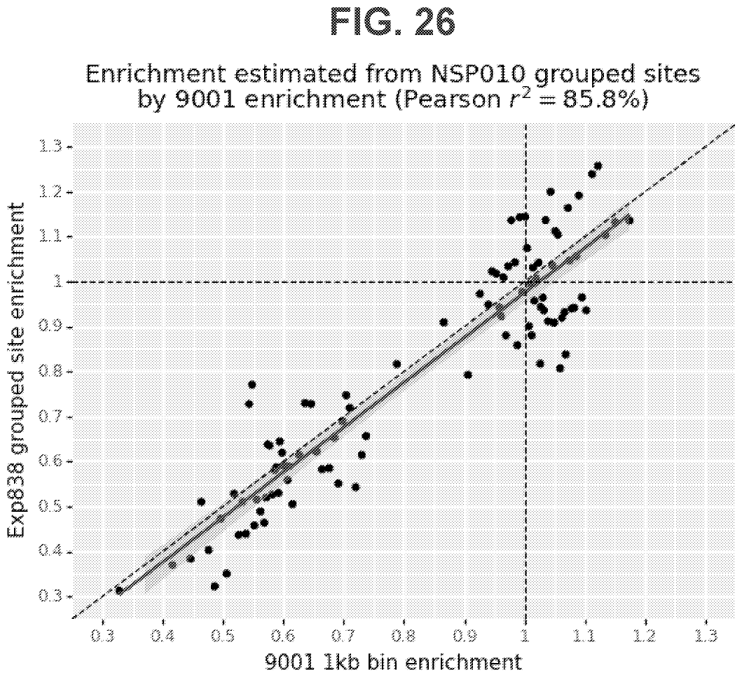

In this approach, samples of whole-genome-amplified cfDNA were prepared following the protocol described above (DNA extraction and generation of molecular counts using whole genome amplification of cfDNA; see FIG. 23). FIG. 24 shows that the ratio of the chromosome Y counts to the uninformative site counts (i.e., sites in the genome that are not maternally or fetally enriched) tightly correlates with the fetal fraction as determined using a control method. This indicates that the count data is likely to be reliable. FIG. 25 shows the count data for the 100 best performing probes, compared to the fetal fraction as determined using a control method. The data shows that the fetal fraction can be estimated even using a small subset of negative predictor sites. The data was additionally compared with the discovery data (Example 2). As shown on FIG. 26, this comparison revealed that the maternal enrichment estimated from the discovery data (x axis) could be reproduced using targeted data (y axis).

However, the overall process was noisy, likely due to the whole genome amplification step (which was performed in order to increase the amount of starting material). This complicated the interpretation of the data. Thus, the analysis was repeated using pooled cfDNA samples (prepared as described below). The above protocol (DNA extraction and generation of molecular counts using whole genome amplification of cfDNA) was used in these experiments as well, excluding the whole genome amplification process. Instead, cfDNA preparations obtained in Step 1 were pooled for all samples with similar (known) fetal fractions. This allowed to increase the amount of input without increasing the noise as much as when applying WGA (and thus increasing the signal-to-noise ratio).

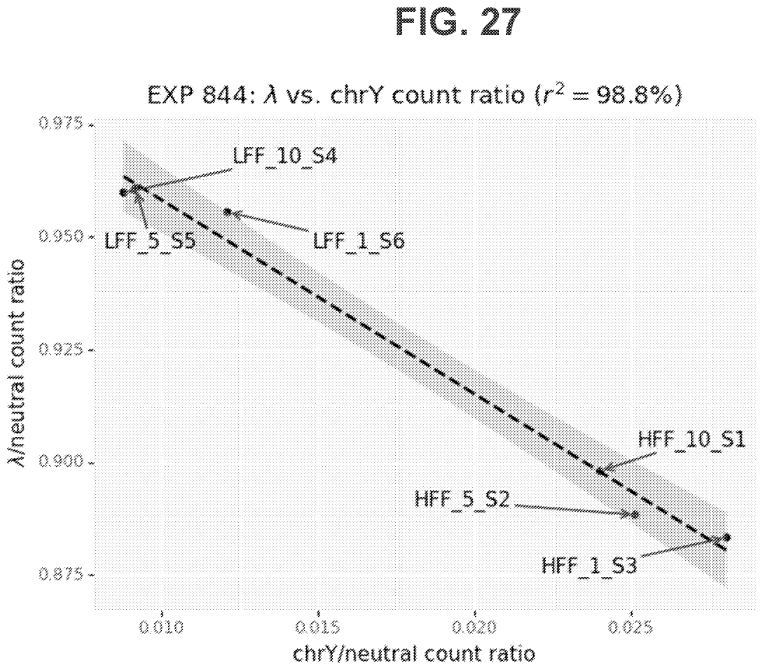

Using pooled cfDNA samples with similar fetal fractions instead of whole-genome-amplified samples, reproducible results could be obtained, as shown on FIG. 27. Indeed, strong and clean dependence of signal depletion on the fetal fraction was observed. FIG. 27 shows the results for pooled samples containing 10 nanograms (LFF_10_S1), 5 nanograms (LFF_5_S1), and 1 nanograms (LFF_1_S1) of cfDNA at the 5% fetal fraction, and pooled samples containing 10 nanograms (HFF_10_S1), 5 nanograms (HFF_5_S1), and 1 nanograms (HFF_1_S1) of cfDNA at the 16% fetal fraction. These results clearly demonstrate that all the considered genomic sites targeted by the designed MIPs exhibit significant signal reduction with increasing fetal fraction. However, significant sample-to-sample variation of pools suggests that some of the genomic locations may have low signal reproducibility (data not shown).

All literature and similar materials cited in this application, including but not limited to, patents, patent applications, articles, books, treatises, manufacturer's instructions, product enclosures, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

We claim:

1. A method for estimating the fetal fraction in a mixed sample comprising fetal and maternal DNA, the method comprising:
obtaining molecular counts for a plurality of predetermined genomic regions, the plurality of genomic regions comprising a first set of regions, wherein regions in the first set of regions are selected such that molecular counts from mixed samples comprising fetal and maternal DNA for these regions are significantly associated with fetal fraction according to a statistical model; and
estimating the fetal fraction in the mixed sample using the molecular counts for the first set of regions and a statistical model that models the molecular counts or variables derived therefrom as predictor variables, and the fetal fraction as response variable.

2. The method of claim 1, wherein the plurality of predetermined genomic regions further comprise a second set of regions, wherein regions in the second set of regions are chosen such that molecular counts from mixed samples comprising fetal and maternal DNA for these regions are not significantly associated with fetal fraction according to a statistical model.

3. The method of claim 2, wherein the step of estimating the fetal fraction in the mixed sample uses the molecular counts from the first and second sets of genomic regions, optionally wherein the statistical model uses one or more ratios of molecular counts from the first and second sets of genomic regions as predictor variables.

4. The method of any preceding claim further comprising the step of obtaining a mixed sample comprising fetal and maternal DNA, wherein the sample is a maternal blood sample, and/or wherein the method further comprises the step of extracting cell free DNA from a mixed sample comprising fetal and maternal DNA.

5. The method of any preceding claim, wherein the step of obtaining molecular counts for a plurality of predetermined genomic regions comprises the step of selectively interrogating target nucleic acids in the sample that can be attributed to the predetermined regions, or wherein the step of obtaining molecular counts for a plurality of predetermined genomic regions comprises receiving or extracting molecular count data for the plurality of predetermined genomic regions, optionally wherein the molecular count data for the plurality of predetermined genomic regions comprises counts for a plurality of target nucleic acids in the sample that can be attributed to the predetermined region.

6. The method of any preceding claim, wherein the step of obtaining molecular counts for a predetermined genomic region comprises combining molecular counts for one or more target nucleic acids located within the predetermined genomic region.

7. The method of any preceding claim, wherein the step of obtaining molecular counts for a first or second set of predetermined genomic regions comprises combining molecular counts for a plurality of target nucleic acids located within any of the first or second set of predetermined regions, or within a subset of the first or second set of predetermined regions, and/or wherein the step of obtaining molecular counts for a first or second set of predetermined genomic regions comprises combining molecular counts for a plurality of target nucleic acids located within a plurality of regions within the first or second set of predetermined regions, optionally using a weighting factor for each of the plurality of regions.

8. The method of any preceding claim, wherein the first set of predetermined genomic regions comprises a plurality of subsets of regions, wherein regions within a subset have a more similar level of association with fetal fraction than regions in different subsets, and wherein the step of obtaining molecular counts for the first set of predetermined genomic regions comprises combining molecular counts for a plurality of target nucleic acids located within respective subsets of the first set of predetermined regions.

9. The method of any preceding claim, wherein the molecular counts have been obtained using a molecular counting method selected from: digital counting assays, microarrays, and targeted sequencing, such as e.g., sequencing of a selectively captured population of nucleic acid molecules.

10. The method of any preceding claim, wherein the first set of regions each have a size individually chosen between approximately 10 bases and approximately 100 kb, or between approximately 100 bases and approximately 10 kb, such as around 1 kb.

11. The method of any preceding claim, wherein the statistical model used in the step of estimating the fetal fraction is a generalized linear model, and/or a model that has been previously trained using training data obtained from samples with known fetal fraction.

12. The method of any preceding claim, wherein the statistical model used to select regions in the first set of regions models the expected molecular count for a region in the genome as the product of:
the total number of counts obtained from a mixed sample from sites with known ploidy, and
a region enrichment factor that is expressed as a weighted combination of a maternal enrichment factor, with weight equal to (1-fetal fraction) and a fetal enrichment factor, with weight equal to the fetal fraction.

13. The method of claim 12, wherein the expected molecular count for a region in the genome is assumed to have a Poisson distribution, a negative binomial distribution or a normal distribution.

14. The method of any preceding claim, further comprising the step of selecting the regions in the first set of regions may be selected by:
(i) fitting a statistical model to molecular counts from a set of mixed samples comprising fetal and maternal DNA for a plurality of candidate regions (where the candidate regions may e.g., represent the entire genome), wherein the statistical model comprises a site-specific fetal enrichment factor and a site-specific maternal enrichment factor for each candidate region, and a fetal fraction for each sample as parameters of the model, and
(ii) determining whether a candidate region is significantly associated with fetal fraction according to the statistical model by comparing the site-specific fetal enrichment factor and the site-specific maternal enrichment factor estimated for the site through the fitting of the model.

15. The method of any of claims 12 to 14, wherein the step of selecting regions in the first set of regions comprises determining the differential enrichment effect size for a candidate region as the difference or absolute difference between the site-specific fetal enrichment factor and the site-specific maternal enrichment factor for the candidate region.

16. The method of any of claims 12 to 16, wherein the step of selecting candidate regions comprises selecting candidate regions that satisfy one or more criteria selected from: the site-specific fetal enrichment factor being significantly different from the site-specific maternal enrichment factor, differential enrichment effect size being above a predetermined threshold and the candidate region being in the set of regions that has the highest significance, such as e.g., the 100, 1000, 2000, 4000, 5000, or 10,000 regions that have the most significant differential effect size amongst the candidate regions tested.

17. A method of providing an assay for estimating fetal fraction, the method comprising:
obtaining molecular counts for a plurality of candidate genomic regions from a set mixed samples comprising maternal and fetal DNA and having known fetal fraction, and selecting a first set of regions by:
(i) fitting a statistical model to the molecular counts, wherein the statistical model comprises a site-specific fetal enrichment factor and a site-specific maternal enrichment factor for each candidate region, and a fetal fraction for each sample as parameters of the model, and
(ii) determining whether a candidate region is significantly associated with fetal fraction according to the statistical model by comparing the site-specific fetal enrichment factor and the site-specific maternal enrichment factor estimated for the site through the fitting of the model, wherein sites in the first set of regions are significantly associated with fetal fraction.

18. The method of claim 17, further comprising identifying a set of target nucleic acids that are located in the first set of regions, and designing an assay that produces molecular counts for these set of target nucleic acids.

19. The method of claim 17 or claim 18, further comprising applying the assay to one or more test samples, each associated with a known or estimated fetal fraction, and identifying target nucleic acids that are associated with comparatively low variability counts between samples with similar fetal fractions.

20. The method of claim 19, wherein identifying target nucleic acids that are associated with comparatively low variability counts between samples with similar fetal fractions comprises combining the molecular counts for a candidate target nucleic acid sequence (or candidate set of target nucleic acid sequences) in each of a plurality of groups of samples that have similar known or estimated fetal fractions, and obtaining a measure of molecular count variability within the groups.

21. The method of any preceding claim, wherein molecular counts are not allele specific.

22. The method of any preceding claim, wherein the regions in the first set of regions are located on autosomes.

23. The method of any preceding claim, wherein the regions in the first set of regions are significantly negatively associated with fetal fraction.

24. The method of any of claims 12 to 23, wherein the regions in the first set of regions have an enrichment ratio of between 0.7 and 0.9, wherein the enrichment ratio is defined as the ratio between the site-specific fetal enrichment factor and the site-specific maternal enrichment factor.

25. The method of any of claims 12 to 24, wherein the regions in the first set of regions have an average enrichment ratio of approximately 0.8, wherein the enrichment ratio is defined as the ratio between the site-specific fetal enrichment factor and the site-specific maternal enrichment factor.

26. The method of any preceding claim, wherein the regions in the first and/or second set of regions have molecular counts with low variability across a set of mixed samples within a predetermined range of known fetal fraction.

27. The method of any preceding claim, wherein the first set of regions comprises between 2,000 and 20,000 regions.

28. The method of any preceding claim, wherein the molecular counts have been obtained, or wherein obtaining the molecular counts comprises: using a molecular counting method comprising selectively capturing a population of nucleic acid molecules associated with the plurality of regions using capture probes, optionally amplifying the captured population of nucleic acid molecules, and counting the molecules in the captured population of nucleic acid molecules or the amplification products derived therefrom, wherein the capture probes are molecular inversion probes and/or wherein the method comprises obtaining rolling circle amplification products from the captured population of nucleic acid molecules.

29. The method of claim 28, wherein counting the molecules in the captured population of nucleic acid molecules or the amplification products derived therefrom comprises sequencing the captured population of nucleic acid molecules or the amplification products derived therefrom, or wherein counting the molecules in the captured population of nucleic acid molecules or the amplification products derived therefrom comprises labelling the captured population of nucleic acid molecules or the amplification products derived therefrom and counting the labelled molecules.

30. The method of any preceding claim, wherein the regions in the first set of predetermined genomic regions are selected such that the sensitivity of the fetal fraction estimate is at least 0.8, at least 0.85, or at least 0.9, for every fetal fraction within a predetermined range of fetal fractions.

31. The method of any preceding claim, wherein the regions in the first set of predetermined regions comprises a plurality of genomic regions with genes that are differentially expressed or measurable by molecular counting in trophoblast cells and in a plurality of types of maternal cells.

32. The method of claim 31, wherein the regions in the first set of predetermined regions comprises a plurality of genomic regions with genes that are more highly expressed or measurable by molecular counting in trophoblast than in one or more types of maternal cells.

33. The method of any preceding claims, wherein the regions in the first set of predetermined regions comprises a plurality of genomic regions that are enriched for DNAse H sensitive sites in the placenta, compared to other candidate regions.

34. A method for diagnosing a fetal chromosomal abnormality using a mixed sample from a subject, the mixed sample comprising fetal DNA and maternal DNA, the method comprising:
obtaining molecular counts for a plurality of predetermined genomic regions in the mixed sample, the plurality of genomic regions comprising a first set of regions, wherein regions in the first set of regions are selected such that molecular counts from mixed samples comprising fetal and maternal DNA for these regions are significantly associated with fetal fraction according to a statistical model, and at least a second set of regions, wherein regions in the second set of regions are regions that are associated with of one or more fetal chromosomal abnormalities to be identified;
estimating the fetal fraction in the mixed sample using the molecular counts for the first set of regions and a statistical model that models the molecular counts or variables derived therefrom as predictor variables, and the fetal fraction as response variable;
estimating whether a fetal chromosomal abnormality is likely in view of the molecular counts for regions in the second set of regions and the fetal fraction estimate.

35. A method of preparing a set of circularized nucleic acid probes, the method comprising:
a) providing a set of molecular inversion probes (MIPs) designed to selectively interrogate a set of target nucleic acids located in a first set of regions selected according to any of claims 17-33, wherein in the presence of the target nucleic acids, the MIPs are ligatable to form circularized nucleic acid probes;
b) exposing the set of MIPs to a mixed sample comprising fetal and maternal DNA in a ligation mixture wherein MIPs are ligated to form circularized nucleic acid probes.

36. A method of preparing a plurality countable products, comprising:
i) forming a plurality of complexes, each complex comprising an oligonucleotide primer hybridized to a circularized nucleic acid probe produced according to claim 35; and
ii) extending primers in the complexes in one or more rolling circle amplification (RCA) reactions to form countable RCA products that comprise primer portions.

37. The method of claim 36, wherein the primers or primer portions are localized to dispersed loci and wherein:
a) the primers or primer portions are bound to one or more surfaces, preferably covalently linked to the one or more surfaces, or
b) the primers or primer portions are hybridized to capture oligonucleotides, wherein the capture oligonucleotides are bound to one or more surfaces, preferably covalently linked to the one or more surfaces.

38. The method of claim 37, wherein the one or more surfaces are selected from a portion of an assay plate, preferably a multi-well assay plate, preferably a glass-bottom assay plate; a portion of a slide; and one or more particles, preferably nanoparticles, wherein the particles are preferably paramagnetic particles, preferably ferromagnetic nanoparticles, preferably iron oxide nanoparticles.

39. The method of claim 38, wherein the primers or primer portions are bound to surfaces on particles, wherein the RCA products are localized to dispersed loci by one or more of a magnet, centrifugation, and filtration.

40. The method of any one of claims 37-39, wherein the dispersed loci are in an irregular dispersal or wherein the dispersed loci are in an addressable array.
